(19) United States
(12) Patent Application Publication
(10) Pub. No.: US 2026/0139033 A1
(43) Pub. Date: May 21, 2026

(54) TSLP INHIBITORS AND METHODS OF USE THEREOF

(71) Applicant: Prana Therapies, Inc., Encinitas, CA (US)

(72) Inventors: Miguel A. SANJUAN, La Jolla, CA (US); Sheila GUJRATHI, Encinitas, CA (US)

(21) Appl. No.: 19/368,398

(22) Filed: Oct. 24, 2025

Related U.S. Application Data
(63) Continuation of application No. PCT/US24/26539, filed on Apr. 26, 2024.
(60) Provisional application No. 63/499,139, filed on Apr. 28, 2023.
(60) Provisional application No. 63/633,923, filed on Apr. 15, 2024.

(51) Int. Cl. C07K 14/715; C07K 16/28

(52) U.S. Cl. CPC C07K 14/7155; C07K 16/2866; C07K 2317/31; C07K 2317/52; C07K 2317/92; C07K 2317/94; C07K 2319/00

(57) ABSTRACT

TSLP-TRAPs are a novel class of biologics that have been developed to inhibit the activity of TSLP. By binding to TSLP, TSLP-TRAPS effectively block the interaction between TSLP and its receptor, preventing downstream signaling and immune cell activation. This makes TSLP-TRAPs an attractive therapeutic option for a variety of inflammatory and allergic diseases, including asthma, atopic dermatitis, and eosinophilic esophagitis.

### CROSS-REFERENCE

This application is a continuation of International Application No. PCT/US24/26539, filed Apr. 26, 2024, which claims the benefit of U.S. Provisional Application No. 63/499,139, filed Apr. 28, 2023, and U.S. Provisional Application No. 63/633,923, filed Apr. 15, 2024, each of which is hereby incorporated by reference in its entirety.

### SEQUENCE LISTING

The instant application contains a Sequence Listing which has been submitted electronically in XML format and is hereby incorporated by reference in its entirety. Said XML copy, created on Jan. 30, 2026, is named 62413-702301.xml and is 75,196 bytes in size.

### BACKGROUND OF THE INVENTION

TSLP, or thymic stromal lymphopoietin, is a cytokine that plays a crucial role in the initiation and modulation of immune responses. It is produced by several cell types, including epithelial cells, fibroblasts, and dendritic cells, and has been implicated in the development of a wide range of inflammatory and allergic diseases. TSLP functions by binding to its receptor TSLPR, which is expressed on various immune cells, including T cells, dendritic cells and B cells. Once bound, TSLP-TSLPR signaling triggers a cascade of downstream events that ultimately result in the activation of immune cells and the release of proinflammatory cytokines. Verstraete, K., et. al., “Structure and antagonism of the receptor complex mediated by human TSLP in allergy and asthma” Nature Communications., 8: art.n° 14937. (2017).

TSLP-TRAPs are a novel class of biologics that have been developed to inhibit the activity of TSLP. The TSLP-TRAP can be a protein heterodimer comprising an extracellular domain of thymic stromal lymphopoietin protein receptor (TSLPR) and an extracellular domain of interleukin-7 receptor subunit alpha (IL-7Ra). In some cases, the TSLP-TRAP comprises a protein heterodimer comprising an extracellular domain of TSLPR and an extracellular domain of IL-7Rα joined via a second linker. In some cases, the TSLP-TRAP comprises fusing an extracellular domain of TSLPR and an extracellular domain of IL-7Rα via a linker. By binding to TSLP, TSLP-TRAPS effectively block the interaction between TSLP and its receptor, preventing downstream signaling and immune cell activation. This makes TSLP-TRAPs an attractive therapeutic option for a variety of inflammatory and allergic diseases, including asthma, atopic dermatitis, and eosinophilic esophagitis. The monospecific TSLP-TRAP complexes have demonstrated efficacy in sequestering TSLP. Furthermore, the present disclosure includes polyspecific TSLP-TRAP complexes that bind additional targets such as anti-IL-4R TSLP-TRAP complexes having demonstrated efficacy in blocking IL-4R and sequestering TSLP.

### SUMMARY OF THE INVENTION

Provided herein is an antigen binding complex comprising a TSLP-TRAP associated with a moiety via a first linker. In some embodiments, the TSLP-TRAP comprises an extracellular domain of thymic stromal lymphopoietin protein receptor (TSLPR) and an extracellular domain of interleukin-7 receptor subunit alpha (IL-7Rα) joined via a second linker or independently joined to the moiety via a second linker, a first linker or a linker disclosed herein. In some embodiments, the TSLP-TRAP comprises an extracellular domain of TSLPR and an extracellular domain of IL-7Rα joined via a second linker. In some embodiments, the TSLPR is joined to the moiety via the first linker, the second linker or a linker disclosed herein. In some embodiments, the IL-7Rα is joined to the moiety via the first linker, the second linker or a linker disclosed herein. In some embodiments, the TSLPR is joined to a first portion of the moiety and the IL-7Rα is joined to a second portion of the moiety. In some embodiments, the TSLPR is a human TSLPR. In some embodiments, the extracellular domain of TSLPR comprises a sequence having at least 90% sequence identity to an amino acid sequence set out in SEQ ID NO: 1. In some embodiments, the extracellular domain of TSLPR comprises an amino acid sequence set out in SEQ ID NO: 1. In some embodiments, the IL-7Rα is a human IL-7Rα. In some embodiments, the extracellular domain of IL-7Rα comprises a sequence having at least 90% sequence identity to an amino acid sequence set out in SEQ ID NO: 2. In some embodiments, the extracellular domain of IL-7Rα comprises an amino acid sequence set out in SEQ ID NO: 2. In some embodiments, the second linker is a polypeptide between 4 amino acids and 500 amino acids in length that associates the extracellular domain of TSLPR to the extracellular domain of IL-7Rα. In some embodiments, the second linker is a polypeptide at least 60 amino acids in length that associates the extracellular domain of TSLPR to the extracellular domain of IL-7Rα. In some embodiments, the second linker is a polypeptide at most 60) amino acids in length that associates the extracellular domain of TSLPR to the extracellular domain of IL-7Rα. In some embodiments, the second linker is a polypeptide 60 amino acids in length that associates the extracellular domain of TSLPR to the extracellular domain of IL-7Rα. In some embodiments, the second linker is a polypeptide at least 20 amino acids in length that associates the extracellular domain of TSLPR to the extracellular domain of IL-7Rα. In some embodiments, the second linker is a polypeptide at most 20 amino acids in length that associates the extracellular domain of TSLPR to the extracellular domain of IL-7Rα. In some embodiments, the second linker is a polypeptide 20 amino acids in length that associates the extracellular domain of TSLPR to the extracellular domain of IL-7Rα.

In some embodiments, the second linker comprises (GGS)n, wherein n is an integer between 1 and 100. In some embodiments, the second linker comprises (GGS) x20 (SEQ ID NO: 3). In some embodiments, the TSLP-TRAP comprises amino acids 25-231 of human TSLPR associated via (GGS)x20 (SEQ ID NO: 3) to amino acids 21-239 of human IL-7Rα. In some embodiments, the TSLP-TRAP comprises amino acids 21-239 of human IL-7Rα associated via (GGS)x20 (SEQ ID NO: 3) to amino acids 25-231 of human TSLPR. In some embodiments, the TSLP-TRAP comprises a sequence having at least 90% sequence identity to an amino acid sequence set out in SEQ ID NO: 4. In some embodiments, the TSLP-TRAP comprises an amino acid sequence set out in SEQ ID NO: 4. In some embodiments, the TSLP-TRAP comprises a sequence having at least 90% sequence identity to an amino acid sequence set out in SEQ ID NO: 5. the TSLP-TRAP comprises a sequence having at least 90% sequence identity to an amino acid sequence set out in SEQ ID NO: 5. In some embodiments, the TSLP-TRAP comprises an amino acid sequence set out in SEQ ID NO: 5. In some embodiments, the first linker is a polypeptide between 4 amino acids and 500 amino acids in length that associates the TSLP-TRAP to the moiety.

In some embodiments, the first linker or second linker is a polypeptide at least 60 amino acids in length that associates the TSLP-TRAP to the moiety. In some embodiments, the first linker or second linker is a polypeptide at most 60 amino acids in length that associates the TSLP-TRAP to the moiety. In some embodiments, the first linker or second linker is a polypeptide 60 amino acids in length that associates the TSLP-TRAP to the moiety. In some embodiments, the first linker or second linker is a polypeptide at least 20 amino acids in length that associates the TSLP-TRAP to the moiety. In some embodiments, the first linker or second linker is a polypeptide at most 20 amino acids in length that associates the TSLP-TRAP to the moiety. In some embodiments, the first linker or a second linker is a polypeptide 20 amino acids in length that associates the TSLP-TRAP to the moiety.

In some embodiments, the moiety is connected to the first linker, second linker, or a linker disclosed herein. In some embodiments, the first or second linker is a polypeptide 60 amino acids in length that associates the moiety to the TSLPR. In some embodiments, the first or second linker is a polypeptide at most 60 amino acids in length that associates the moiety to the TSLPR. In some embodiments, the first or second linker is a polypeptide at least 60 amino acids in length that associates the moiety to the TSLPR. In some embodiments, the first or second linker is a polypeptide 20 amino acids in length that associates the moiety to the TSLPR. In some embodiments, the first or second linker is a polypeptide at most 20 amino acids in length that associates the moiety to the TSLPR. In some embodiments, the first or second linker is a polypeptide at least 20 amino acids in length that associates the moiety to the TSLPR. In some embodiments, the first or second linker is a polypeptide 60 amino acids in length that associates the moiety to the IL-7Rα. In some embodiments, the first or second linker is a polypeptide at most 60 amino acids in length that associates the moiety to the IL-7Rα. In some embodiments, the first or second linker is a polypeptide at least 60 amino acids in length that associates the moiety to the IL-7Rα. In some embodiments, the first or second linker is a polypeptide 20 amino acids in length that associates the moiety to the IL-7Rα. In some embodiments, the first or second linker is a polypeptide at most 20 amino acids in length that associates the moiety to the IL-7Rα. In some embodiments, the first or second linker is a polypeptide at least 20 amino acids in length that associates the moiety to the IL-7Rα. In some embodiments, the first or second linker is a polypeptide 60 amino acids in length that associates the IL-7Rα to the TSLPR. In some embodiments, the first or second linker associates is a polypeptide at least 60 amino acids in length that associates the IL-7Rα to the TSLPR. In some embodiments, the first or second linker is a polypeptide at most 60 amino acids in length that associates the IL-7Rα to the TSLPR. In some embodiments, the first or second linker is a polypeptide 20 amino acids in length that associates the IL-7Rα to the TSLPR. In some embodiments, the first or second linker is a polypeptide at least 20 amino acids in length that associates the IL-7Rα to the TSLPR. In some embodiments, the first or second linker is a polypeptide at most 20 amino acids in length that associates the IL-7Rα to the TSLPR.

In some embodiments, the first linker comprises an amino acid sequence having at least 90% sequence identity to an amino acid sequence of (GGGGS)n (SEQ ID NO: 40), wherein n is an integer between 1 and 100. In some embodiments, the first linker comprises an amino acid sequence having at least 90% sequence identity to an amino acid sequence of (GGGGS)x4 (SEQ ID NO: 6). In some embodiments, the amino acid sequence of the first linker comprises (GGGGS)x4 (SEQ ID NO: 6). In some embodiments, the first linker is flexible or rigid. In some embodiments, the first linker is cleavable or non-cleavable. In some embodiments, the antigen binding complex comprises at least two TSLP-TRAP. In some embodiments, the at least two TSLP-TRAP is each separately associated with the moiety via a linker. In some embodiments, a TSLP-TRAP is associated with a second TSLP-TRAP. In some embodiments, the at least two TSLP-TRAP comprises 3 TSLP-TRAPs. In some embodiments, the at least two TSLP-TRAP comprises 4 TSLP-TRAPs. In some embodiments, the moiety is selected from the group consisting of: 1) a probe: 2) an antibody domain: 3) an antibody or antigen binding portion thereof: 4) a receptor; and 5) a half-life extender. In some embodiments, the moiety is the probe, wherein the probe is a His tag, a GST tag, a flag tag, or a fluorescent molecule. In some embodiments, the moiety is the antibody domain, wherein the antibody domain comprises a Fc region. In some embodiments, the Fc region is an IgG1, IgG2, IgG3, or IgG4 heavy chain Fc region. In some embodiments, the Fc region comprises M252Y/S254T/T256E (YTE), M428L/N434S (LS), T307A/E380A/N434A (AAA), T250Q/M428L (QL), or V308P sequence mutations. In some embodiments, the moiety is the antibody or antigen binding portion thereof. In some embodiments, the antibody or antigen binding portion thereof is a monoclonal antibody, a bispecific antibody, a Fab, a Fab′, a Fv, a scFV, or a (Fab′)₂. In some embodiments, the antibody or antigen binding portion thereof comprises IgG1, IgG2, IgG3, or IgG4. In some embodiments, the antibody or antigen binding portion thereof specifically binds human interleukin-4 receptor (hIL-4R), human interleukin-4 (hIL-4), human interleukin-13 receptor (hIL-13R), human interleukin-13 (hIL-13), human interleukin-5 (hIL-5), human interleukin-5 receptor (hIL-5R), human interleukin-33 (hIL-33) or human interleukin-31 (hIL-31). In some embodiments, the TSLP-TRAP is associated with a C terminus or termini of the moiety. In some embodiments, the TSLP-TRAP is associated with a N terminus or termini of the moiety. In some embodiments, the moiety is a receptor. In some embodiments, the moiety is a half-life extender. In some embodiments, the half-life extender comprises an antibody or antigen binding portion that is modified to extend the complex's half-life in blood. In some embodiments, the half-life extender is albumin, PEG, or a suitable polymer to extend half-life in blood. In some embodiments, the antibody or antigen-binding portion thereof specifically binds human interleukin-4 receptor (hIL-4R). In some embodiments, the antibody or antigen binding portion thereof comprises (a) a HCDR1 having at least 80%, 90%, 95%, 98% sequence identity to the amino acid sequence set out in SEQ ID NO: 7, (b) a HCDR2 having the amino acid sequence set out in SEQ ID NO: 8, and/or (c) a HCDR3 having at least 80%, 90%, 95%, 98% sequence identity to the amino acid sequence set out in SEQ ID NO: 9. In some embodiments, the antibody or antigen binding portion thereof comprises (a) a HCDR1 comprising an amino acid sequence of SEQ ID NO: 7. (b) a HCDR2 comprising an amino acid sequence of SEQ ID NO: 8, and/or (c) a HCDR3 comprising an amino acid sequence set out in SEQ ID NO: 9. In some embodiments, the antibody or antigen binding portion thereof comprises a heavy chain variable domain having at least 80%, 90%, 95%, 98% sequence identity to the amino acid sequence set out in SEQ ID NO: 10. In some embodiments, the antibody or antigen binding portion thereof comprises a heavy chain variable domain comprising an amino acid sequence out in SEQ ID NO: 10. In some embodiments, the antibody or antigen binding portion thereof comprises a heavy chain having at least 80%, 90%, 95%, 98% sequence identity to the amino acid sequence set out in SEQ ID NO: 11. In some embodiments, the antibody or antigen binding portion thereof comprises a heavy chain Fc region comprising M252Y/S254T/T256E (YTE), M428L/N434S (LS), T307A/E380A/N434A (AAA), T250Q/M428L (QL), or V308P sequence mutations. In some embodiments, the antibody or antigen binding portion thereof disclosed herein comprises S228P modification that stabilizes the hinge region. In some embodiments, the antibody or antigen binding portion thereof comprises a heavy chain comprising an amino acid sequence set out in SEQ ID NO: 11. In some embodiments, the antibody or antigen binding portion thereof comprises (a) a LCDR1 having at least 80%, 90%, 95%, 98% sequence identity to the amino acid sequence set out in SEQ ID NO: 12, (b) a LCDR2 having the amino acid sequence set out in SEQ ID NO: 13, and/or (c) a LCDR3 having at least 80%, 90%, 95%, 98% sequence identity to the amino acid sequence set out in SEQ ID NO: 14. In some embodiments, the antibody or antigen binding portion thereof comprises (a) a LCDR1 comprising an amino acid sequence of SEQ ID NO: 12, (b) a LCDR2 comprising an amino acid sequence of SEQ ID NO: 13, and/or (c) a LCDR3 comprising an amino acid sequence of SEQ ID NO: 14. In some embodiments, the antibody or antigen binding portion thereof comprises a light chain variable domain having at least 80%, 90%, 95%, 98% sequence identity to the amino acid sequence set out in SEQ ID NO: 15. In some embodiments, the antibody or antigen binding portion thereof comprises a light chain variable domain comprising an amino acid sequence set out in SEQ ID NO: 15. In some embodiments, the antibody or antigen binding portion thereof comprises a light chain having at least 80%, 90%, 95%, 98% sequence identity to the amino acid sequence set out in SEQ ID NO: 16. In some embodiments, the antibody or antigen binding portion thereof comprises a light chain comprising an amino acid sequence set out in SEQ ID NO: 16. In some embodiments, the antibody or antigen-binding portion thereof is dupilumab. In some embodiments, the antibody or antigen binding portion thereof further comprises a Fc region with one or more mutations to extend half-life. In some embodiments, the one or more mutations to extend half-life comprises M252Y/S254T/T256E (YTE), M428L/N434S (LS), T307A/E380A/N434A (AAA), T250Q/M428L (QL), or V308P sequence mutations. In some embodiments, the antibody or antigen binding portion thereof does not bind. In some embodiments, the antibody or antigen binding portion thereof does not bind to an endogenous antigen. In some embodiments, the antibody or antigen binding portion thereof comprises (a) a HCDR1 having at least 80%, 90%, 95%, 98% sequence identity to the amino acid sequence set out in SEQ ID NO: 17, (b) a HCDR2 having the amino acid sequence set out in SEQ ID NO: 18, and/or (c) a HCDR3 having at least 80%, 90%, 95%, 98% sequence identity to the amino acid sequence set out in SEQ ID NO: 19. In some embodiments, the antibody or antigen binding portion thereof comprises (a) a HCDR1 comprising an amino acid sequence of SEQ ID NO: 17, (b) a HCDR2 comprising an amino acid sequence of SEQ ID NO: 18, and/or (c) a HCDR3 comprising an amino acid sequence set out in SEQ ID NO: 19. In some embodiments, the antibody or antigen binding portion thereof comprises a heavy chain variable domain having at least 80%, 90%, 95%, 98% sequence identity to the amino acid sequence set out in SEQ ID NO: 20. In some embodiments, the antibody or antigen binding portion thereof comprises a heavy chain variable domain comprising an amino acid sequence out in SEQ ID NO: 20. In some embodiments, the antibody or antigen binding portion thereof comprises a heavy chain having at least 80%, 90%, 95%, 98% sequence identity to the amino acid sequence set out in SEQ ID NO: 21. In some embodiments, the antibody or antigen binding portion thereof comprises a heavy chain Fc region comprising M252Y/S254T/T256E (YTE), M428L/N434S (LS), T307A/E380A/N434A (AAA), T250Q/M428L (QL), or V308P sequence mutations. In some embodiments, the antibody or antigen binding portion thereof comprises a heavy chain comprising an amino acid sequence set out in SEQ ID NO: 21. In some embodiments, the antibody or antigen binding portion thereof comprises (a) a LCDR1 having at least 80%, 90%, 95%, 98% sequence identity to the amino acid sequence set out in SEQ ID NO: 22, (b) a LCDR2 having the amino acid sequence set out in SEQ ID NO: 23, and/or (c) a LCDR3 having at least 80%, 90%, 95%, 98% sequence identity to the amino acid sequence set out in SEQ ID NO: 24. In some embodiments, the antibody or antigen binding portion thereof comprises (a) a LCDR1 comprising an amino acid sequence of SEQ ID NO: 22, (b) a LCDR2 comprising an amino acid sequence of SEQ ID NO: 23, and/or (c) a LCDR3 comprising an amino acid sequence of SEQ ID NO: 24. In some embodiments, the antibody or antigen binding portion thereof comprises a light chain variable domain having at least 80%, 90%, 95%, 98% sequence identity to the amino acid sequence set out in SEQ ID NO: 25. In some embodiments, the antibody or antigen binding portion thereof comprises a light chain variable domain comprising an amino acid sequence set out in SEQ ID NO: 25. In some embodiments, the antibody or antigen binding portion thereof comprises a light chain having at least 80%, 90%, 95%, 98% sequence identity to the amino acid sequence set out in SEQ ID NO: 26. In some embodiments, the antibody or antigen binding portion thereof comprises a light chain comprising an amino acid sequence set out in SEQ ID NO: 26. In some embodiments, the antibody or antigen-binding portion thereof is palivizumab. In some embodiments, the antibody or antigen binding portion thereof further comprises a Fc region with one or more mutations to extend half-life. In some embodiments, the one or more mutations to extend half-life comprises M252Y/S254T/T256E (YTE), M428L/N434S (LS), T307A/E380A/N434A (AAA), T250Q/M428L (QL), or V308P sequence mutations. In some embodiments, the antigen binding complex comprises a sequence having at least 80%, 90%, 95%, 98% sequence identity to an amino acid sequence set out in SEQ ID NO: 27. In some embodiments, the antigen binding complex comprises an amino acid sequence of SEQ ID NO: 27. In some embodiments, the antigen binding complex comprises a sequence having at least 80%, 90%, 95%, 98% sequence identity to an amino acid sequence set out in SEQ ID NO: 28. In some embodiments, the antigen binding complex comprises an amino acid sequence of SEQ ID NO: 28. In some embodiments, the antigen binding complex comprises a sequence having at least 80%, 90%, 95%, 98% sequence identity to an amino acid sequence set out in SEQ ID NO: 29. In some embodiments, the antigen binding complex comprises an amino acid sequence of SEQ ID NO: 29. In some embodiments, the antigen binding complex comprises a sequence having at least 80%, 90%, 95%, 98% sequence identity to an amino acid sequence set out in SEQ ID NO: 30. In some embodiments, the antigen binding complex comprises an amino acid sequence of SEQ ID NO: 30. In some embodiments, the antigen binding complex comprises a sequence having at least 80%, 90%, 95%, 98% sequence identity to an amino acid sequence set out in SEQ ID NO: 31. In some embodiments, the antigen binding complex comprises an amino acid sequence set out in SEQ ID NO: 31. In some embodiments, the antigen binding complex comprises a sequence having at least 80%, 90%, 95%, 98% sequence identity to an amino acid sequence set out in SEQ ID NO: 32. In some embodiments, the antigen binding complex comprises an amino acid sequence of SEQ ID NO: 32. In some embodiments, the antigen binding complex comprises a sequence having at least 80%, 90%, 95%, 98% sequence identity to an amino acid sequence set out in SEQ ID NO: 33. In some embodiments, the antigen binding complex comprises an amino acid sequence of SEQ ID NO: 33. In some embodiments, the antigen binding complex comprises a sequence having at least 80%, 90%, 95%, 98% sequence identity to an amino acid sequence set out in SEQ ID NO: 34. In some embodiments, the antigen binding complex comprises an amino acid sequence of SEQ ID NO: 34. In some embodiments, the antigen binding complex comprises a sequence having at least 80%, 90%, 95%, 98% sequence identity to an amino acid sequence set out in SEQ ID NO: 35. In some embodiments, the antigen binding complex comprises an amino acid sequence of SEQ ID NO: 35. In some embodiments, the antigen binding complex comprises a sequence having at least 80%, 90%, 95%, 98% sequence identity to an amino acid sequence set out in SEQ ID NO: 36. In some embodiments, the antigen binding complex comprises an amino acid sequence of SEQ ID NO: 36. In some embodiments, the antigen binding complex comprises a sequence having at least 80%, 90%, 95%, 98% sequence identity to an amino acid sequence set out in SEQ ID NO: 37. In some embodiments, the antigen binding complex comprises an amino acid sequence of SEQ ID NO: 37. In some embodiments, the antigen binding complex comprises a sequence having at least 80%, 90%, 95%, 98% sequence identity to an amino acid sequence set out in SEQ ID NO: 38. In some embodiments, the antigen binding complex comprises an amino acid sequence of SEQ ID NO: 38. In some embodiments, the antigen binding complex comprises a sequence having at least 80%, 90%, 95%, 98% sequence identity to an amino acid sequence set out in SEQ ID NO: 39. In some embodiments, the antigen binding complex comprises an amino acid sequence of SEQ ID NO: 39. In some embodiments, the antigen binding complex comprises a sequence having at least 80%, 90%, 95%, 98% sequence identity to an amino acid sequence set out in SEQ ID NO: 45. In some embodiments, the antigen binding complex comprises an amino acid sequence of SEQ ID NO: 45. In some embodiments, the antigen binding complex comprises a sequence having at least 80%, 90%, 95%, 98% sequence identity to an amino acid sequence set out in SEQ ID NO: 46. In some embodiments, the antigen binding complex comprises an amino acid sequence of SEQ ID NO: 46. In some embodiments, the antigen binding complex comprises a sequence having at least 80%, 90%, 95%, 98% sequence identity to an amino acid sequence set out in SEQ ID NO: 47. In some embodiments, the antigen binding complex comprises an amino acid sequence of SEQ ID NO: 47. In some embodiments, the antigen binding complex comprises a sequence having at least 80%, 90%, 95%, 98% sequence identity to an amino acid sequence set out in SEQ ID NO: 48. In some embodiments, the antigen binding complex comprises an amino acid sequence of SEQ ID NO: 48. In some embodiments, the antigen binding complex comprises a sequence having at least 80%, 90%, 95%, 98% sequence identity to an amino acid sequence set out in SEQ ID NO: 49. In some embodiments, the antigen binding complex comprises an amino acid sequence of SEQ ID NO: 49. In some embodiments, the antigen binding complex comprises a sequence having at least 80%, 90%, 95%, 98% sequence identity to an amino acid sequence set out in SEQ ID NO: 50. In some embodiments, the antigen binding complex comprises an amino acid sequence of SEQ ID NO: 50. In some embodiments, the TSLP-TRAP binds TSLP with a KD between 0.2×10⁻¹² M and 30×10⁻¹²M. In some embodiments, the TSLP-TRAP binds TSLP with a KD between 0.2×10⁻¹² M and 10×10⁻¹²M. In some embodiments, disclosed herein are polynucleotide encoding the antigen binding complex disclosed herein. In some embodiments, disclosed herein are expression vectors comprising a polynucleotide encoding the antigen binding complex. In some embodiments, disclosed herein are host cells comprising the expression vectors or a polynucleotide encoding the antigen binding complex. Further disclosed herein are method of producing the antigen binding complex disclosed herein. In some embodiments, disclosed herein are pharmaceutical composition comprising the antigen binding complex, the polynucleotide, the expression vector or the host cell disclosed herein. In some embodiments, disclosed herein are method of treating a disease in a subject in need thereof comprising administering to the subject a therapeutically effective amount of the antigen binding complex, the polynucleotide, the expression vector, the host cell or the pharmaceutical composition disclosed herein. In some embodiments, the disease is an inflammatory disease. In some embodiments, the disease is an autoimmune disease. In some embodiments, disclosed herein are method of treating asthma, chronic obstructive pulmonary disease, eczema, and/or nasal polyps in a subject in need thereof comprising administering to the subject a therapeutically effective amount of the antigen binding complex, the polynucleotide, the expression vector, the host cell or the pharmaceutical composition disclosed herein. In some embodiments, disclosed herein are the antigen binding complex, the polynucleotide, the expression vector, the host cell or the pharmaceutical composition disclosed herein for use in a treatment or manufacture of a medicament. In some embodiments, disclosed herein are the antigen binding complex, the polynucleotide, the expression vector, the host cell or the pharmaceutical composition disclosed herein and instructions for use.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows an exemplary schematic illustration of a monospecific TSLP-TRAP complex. The TSLP-TRAP is a protein heterodimer comprising an extracellular domain of thymic stromal lymphopoietin protein receptor (TSLPR) and an extracellular domain of interleukin-7 receptor subunit alpha (IL-7Rα) can be joined via a second linker. Here, the TSLP-TRAP is associated with an antibody or antibody fragment thereof via a first linker, where the antibody or antibody fragment thereof does not bind to anything when the monospecific TSLP-TRAP complex is administered to a subject.

FIGS. 2A-2D show exemplary schematic illustrations of various configurations of an antigen binding complex comprising an anti-IL-4R associated with 2 TSLP-TRAPs. The TSLP-TRAP is associated with C termini of the anti-IL-4R heavy chains (FIG. 2A), N termini of the anti-IL-4R heavy chains (FIG. 2B), C termini of the anti-IL-4R light chains (FIG. 2C), or N termini of the anti-IL-4R light chains (FIG. 2D).

FIG. 3A and FIG. 3B show an exemplary schematic illustration of various configurations of an antigen binding complex comprising an anti-IL-4R associated with 4 TSLP-TRAPs. The TSLP-TRAP may be associated with C termini of the anti-IL4R light and heavy chains (FIG. 3A) or a N termini of the anti-IL4R light and heavy chains (FIG. 3B).

FIG. 4 shows an exemplary schematic illustration of various configurations of an antigen binding complex comprising an anti-IL-4R associated with 8 TSLP-TRAPS.

FIG. 5 shows a table summarizing antigen binding complex yield (mg), percent monomericity, and SPR binding results to TSLP (pM) and IL-4R (nM).

FIG. 6 shows a table summarizing antigen binding complexes supernatant titer (mg/L), yield (mgs), concentration (mg/mL), and purity (% monomer).

FIG. 7 shows a table summarizing antigen binding complex production and stability (supernatant titer (mg/L), yield (mgs), concentration (mg/mL), and purity (% monomer)), alongside SPR binding of IL-4R (pM) and TSLP (pM), alongside biological assays for IL-4R (nM) and TSLP (nM).

FIG. 8 shows a table summarizing antigen binding complex production and stability (supernatant titer (mg/L), yield (mgs), concentration (mg/mL), and purity (% monomer)), alongside SPR binding of IL-4R (pM) and TSLP (pM), alongside biological assays for IL-4R (nM) and TSLP (nM).

FIG. 9 shows ICso curves for dupilumab, Tezepelumab and antigen binding complexes based on CD23 upregulation in response to IL-4 in PBMCs.

FIG. 10 shows ICso curves for dupilumab, Tezepelumab and antigen binding complexes based on TARC secretion in response to TSLP in PBMCs.

FIG. 11A-FIG. 11D show exemplary schematic illustrations of various configurations of an antigen binding complex comprising a TSLP-TRAP. The TSLP-TRAP is associated with N termini (FIG. 11A), C termini (FIG. 11B), N termini (FIG. 11C), or C termini (FIG. 11D) of moieties.

### DETAILED DESCRIPTION OF THE INVENTION

### Overview

Described herein, in some aspects, is an antigen binding complex comprising a TSLP-TRAP associated with a moiety via a first linker. In some aspects, the TSLP-TRAP comprises an extracellular domain of thymic stromal lymphopoietin protein receptor (TSLPR) and an extracellular domain of interleukin-7 receptor subunit alpha (IL-7Rα) joined via a second linker. In some embodiments, the TSLPR is joined to a first portion of the moiety and the IL-7Rα is joined to a second portion of the moiety. In some aspects the TSLPR is a human TSLPR. In some aspects, the first linker is a polypeptide between 4 amino acids and 500 amino acids in length that associates the TSLP-TRAP to the moiety. In some aspects, the amino acid sequence of the first linker comprises (GGGGS)x4 (SEQ ID NO: 6). In some aspects, the second linker is a polypeptide between 4 amino acids and 500 amino acids in length that associates the extracellular domain of TSLPR to the extracellular domain of IL-7Rα. In some aspects, the second linker comprises (GGS)x20 (SEQ ID NO: 3).

TSLP-TRAPs can be associated with a variety of moieties that alter its properties including extending half-life and binding additional target molecules. In some aspects, the moiety is selected from the group consisting of: 1) a probe; 2) an antibody domain; 3) an antibody or antigen binding portion thereof; 4) a receptor; and 5) a half-life extender. In some aspects, the moiety is the probe, wherein the probe is a His tag, a GST tag, a flag tag, or a fluorescent molecule. In some aspects, the moiety is a half-life extender, wherein the half-life extender comprises an Fc modification to extend the complex's half-life. In some aspects, the half-life extender is albumin, PEG, or a suitable polymer to extend half-life in blood.

Cytokines and proinflammatory molecules can play a role in TSLP implicated diseases. Blocking or sequestering these targets increases efficacy of the TSLP-TRAP. Thus, the TSLP-TRAP complex disclosed herein can be monospecific or polyspecific (e.g. bi-specific, tri-specific) binders. In some aspects, the TSLP-TRAP complex is a monospecific binder, only binding to TSLP or an antigen thereof. In some aspects, the TSLP-TRAP complex is a bispecific binder, wherein the TSLP-TRAP complex binds TSLP and the associated antibody or antigen binding portion thereof specifically binds to a an antigen, for example human interleukin-4 receptor (hIL-4R), human interleukin-4 (hIL-4), human interleukin-13 receptor (hIL-13R), human interleukin-13 (hIL-13), human interleukin-5 (hIL-5), human interleukin-5 receptor (hIL-5R), human interleukin-33 (hIL-33) or human interleukin-31 (hIL-31). In some aspects, the antibody or antigen-binding portion thereof that is associated with the TSLP-TRAP is dupilumab. In some aspects, the antibody or antigen binding portion thereof further comprises a Fc region with one or more mutations to extend half-life. The present disclosure includes TSLP-TRAP complexes that bind additional targets such as anti-IL-4R.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The term “a” and “an” refers to one or more (i.e., at least one) of the grammatical object of the article. By way of example, “a polypeptide” encompasses one or more polypeptides.

As used herein, the terms “about” and “approximately,” when used to modify an amount specified in a numeric value or range, indicate that the numeric value as well as reasonable deviations from the value known to the skilled person in the art, for example±20%, ±10%, or ±5%, are within the intended meaning of the recited value.

Unless otherwise specified, a “nucleotide sequence encoding a protein” includes all nucleotide sequences that are degenerate versions of each other and thus encode the same amino acid sequence.

The term “transduced”, “transfected”, or “transformed” refers to a process by which exogenous nucleic acid is introduced or transferred into a cell. A “transduced,” “transfected,” or “transformed” cell (e.g., a mammalian cell, a hepatocyte) is one that has been transduced, transfected or transformed with exogenous nucleic acid (e.g., an expression vector) that includes an exogenous nucleic acid encoding a polypeptide).

The term “subject” is intended to include any mammal. In some embodiments, the subject is a cat, a dog, a goat, a human, a non-human primate, a rodent (e.g., a mouse or a rat), a pig, or a sheep.

The term “nucleic acid” refers to a deoxyribonucleic acid (DNA) or ribonucleic acid (RNA), or a combination thereof, in either a single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

Modifications can be introduced into a nucleotide sequence by standard techniques known in the art, such as site-directed mutagenesis and polymerase chain reaction (PCR)-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., arginine, lysine and histidine), acidic side chains (e.g., aspartic acid and glutamic acid), uncharged polar side chains (e.g., asparagine, cysteine, glutamine, glycine, serine, threonine, tyrosine, and tryptophan), nonpolar side chains (e.g., alanine, isoleucine, leucine, methionine, phenylalanine, proline, and valine), beta-branched side chains (e.g., isoleucine, threonine, and valine), and aromatic side chains (e.g., histidine, phenylalanine, tryptophan, and tyrosine), and aromatic side chains (e.g., histidine, phenylalanine, tryptophan, and tyrosine).

The term “antigen-binding domain” is used to refer to one or more antibody variable domain(s) (e.g., formed from amino acids from a single polypeptide or formed from amino acids from two or more polypeptides (e.g., the same or different polypeptides) that is capable of specifically binding to one or more different antigen(s). In some examples, an antigen-binding domain can bind to an antigen or epitope with specificity and affinity similar to that of naturally-occurring antibodies. In some embodiments, the antigen-binding domain can be an antibody or a fragment thereof. In some embodiments, an antigen-binding domain can include an alternative scaffold. Non-limiting examples of antigen-binding domains are described herein. Additional examples of antigen-binding domains are known in the art.

Unless otherwise specified, a “nucleotide sequence encoding a protein” includes all nucleotide sequences that are degenerate versions of each other and thus encode the same amino acid sequence.

The term “N-terminally positioned” when referring to a position of a first domain or sequence relative to a second domain or sequence in a polypeptide primary amino acid sequence means that the first domain is located closer to the N-terminus of the polypeptide primary amino acid sequence. In some embodiments, there may be additional sequences and/or domains between the first domain or sequence and the second domain or sequence.

The term “C-terminally positioned” when referring to a position of a first domain or sequence relative to a second domain or sequence in a polypeptide primary amino acid sequence means that the first domain is located closer to the C-terminus of the polypeptide primary amino acid sequence. In some embodiments, there may be additional sequences and/or domains between the first domain or sequence and the second domain or sequence.

The term “exogenous” refers to any material introduced from or originating from outside a cell, a tissue, or an organism that is not produced by or does not originate from the same cell, tissue, or organism in which it is being introduced.

The term “endogenous” refers to any material originating from inside a cell, a tissue, or an organism that is not introduced by or does not originate from outside the same cell, tissue, or organism in which it is being introduced.

The term “antibody” refers to a protein with an immunoglobulin fold that specifically binds to an antigen via its variable region or regions. The term “antibody” is used herein in the broadest sense and encompasses monoclonal antibodies, polyclonal antibodies, dimers, multimers, multispecific antibodies (e.g., bispecific antibodies), and antibody fragments, so long as they exhibit the desired biological activity (Miller et al., J. Immunol. 170:4854-4861, 2003). Antibodies may be murine, human, humanized, chimeric, or derived from other species. (Janeway, C., Travers, P., Walport, M., Shlomchik (2001) Immunol. Biology, 5th Ed., Garland Publishing. New York). A target antigen generally has numerous binding sites, also called epitopes, recognized by CDRs (complementarity determining regions) on multiple antibodies. Each antibody that specifically binds to a different epitope may have a different structure. Thus, one antigen may have more than one corresponding antibody. An antibody can, e.g., include a full-length immunoglobulin molecule or an immunologically active portion of a full-length immunoglobulin molecule, i.e., a molecule that contains an antigen-binding site that immunospecifically binds an antigen of a target of interest or part thereof. The immunoglobulin disclosed herein can be of any type (e.g., IgG, IgE, IgM, IgD, and IgA), class (e.g., IgG1, IgG2, IgG3, IgG4, IgAQ1 and IgA2) or subclass of immunoglobulin molecule. The immunoglobulins can be derived from any species. In one aspect, however, the immunoglobulin is of human, murine, or rabbit origin.

Antibodies bound to various types of molecules, such as polyethylene glycols (PEGs), and albumin may be used as modified antibodies. Methods for modifying antibodies are already established in the art.

The term “antibody fragments” refers to a portion of a full-length antibody or a polypeptide that includes a portion of a full-length antibody, that retains antigen-binding activity via its variable region or regions. Examples of antibody fragments include Fab, Fab′, F(ab′)2, and Fv fragments; diabodies; linear antibodies; minibodies (Olafsen et al., Protein Eng. Design & Sel. 17(4):315-323, 2004), fragments produced by a Fab expression library, single-chain antibody molecules; and multispecific antibodies formed from antibody fragments.

The term “complementarity determining region” or “CDR” refers to one of the three hypervariable regions (or HVRs) that are known to confer (at least in part) antigen-binding specificity in each antibody light chain and each antibody heavy. The three CDRs in the antibody heavy chain and the antibody light chain interrupt four framework regions in the heavy chain variable domain and the light chain variable domain. The CDRs of each chain are typically referred to as CDR1, CDR2, and CDR3, numbered sequentially starting from the N-terminus, and are also typically identified by the chain in which the particular CDR is located.

The “framework regions” or “FRs” of different light immunoglobulin chains and different heavy immunoglobulin chains are relatively conserved within different antibodies produced by a mammal. The framework regions of light and heavy immunoglobulin chains serve to position and align the CDRs in three-dimensional space. Framework sequences can be obtained from public DNA databases or published references that include germline antibody gene sequences. For example, germline DNA sequences for human heavy and light chain variable region genes can be found in the “VBASE2” germline variable gene sequence database for human and mouse sequences.

“Monospecific” antigen-binding complex refers to the ability of the antigen-binding complex, such as a TSLP-TRAP, to bind specifically to one epitope. “Bispecific” antigen-binding complex refers to the ability of the antigen-binding complex to bind two different epitopes. “Polyspecific” antigen-binding complex refers to the ability of the antigen-binding complex to bind more than one epitope. In certain embodiments, a polyspecific antigen-binding complex, such as a polyspecific TSLP-TRAP complex, encompasses a bispecific antigen-binding polypeptide. For bispecific and polyspecific antigen-binding complexes provided herein, the epitopes can be on the same antigen, or each epitope can be on a different antigen. Therefore, in certain embodiments, a polyspecific antigen-binding complex provided herein, such as a bispecific TSLP-TRAP complex, binds to two different antigens. In certain embodiments, the polyspecific antigen-binding complex, such as a bispecific TSLP-TRAP complex, binds to different epitopes on one antigen. In certain embodiments, a polyspecific antigen-binding complex provided herein binds to each epitope with a dissociation constant (Kd) of about <1 M, about <100 nM, about <10 nM, about <1 nM, about <0.1 nM, about <0.01 nM, or about <0.001 nM (e.g., about 10⁻⁸ M or less, e.g., from about 10⁻⁸ M to about-13M, e.g., from about 10 9 M to about 10-M).

An “amino acid substitution” refers to the replacement of at least one existing amino acid residue in a predetermined amino acid sequence with another different “replacement” amino acid residue. The replacement residue or residues may be “naturally occurring amino acid residues” (i.e. encoded by the genetic code) and selected from the group consisting of: alanine (Ala); arginine (Arg); asparagine (Asn); aspartic acid (Asp); cysteine (Cys); glutamine (GIn); glutamic acid (Glu); glycine (Gly); histidine (His); isoleucine (He); leucine (Leu); lysine (Lys); methionine (Met); phenylalanine (Phe); proline (Pro); serine (Ser); threonine (Thr); tryptophan (Trp); tyrosine (Tyr); and valine (Val).

The term “administer” refers to a method of polypeptides or compositions to the desired site of biological action. These methods include, but are not limited to, topical delivery, parenteral delivery, intravenous delivery, intradermal delivery, intramuscular delivery, colonic delivery, rectal delivery, or intraperitoneal delivery. In one embodiment, the compositions described herein are administered intravenously.

The term “promoter” means a DNA sequence recognized by enzymes/proteins in a cell (e.g., a mammalian cell, a hepatocyte) required to initiate the transcription of an operably linked coding sequence (e.g., a nucleic acid encoding a polypeptide (e.g., any of the exemplary polypeptides described herein). A promoter typically refers, to e.g. a nucleotide sequence to which an RNA polymerase and/or any associated factor binds and at which transcription is initiated. The promoter can be constitutive, inducible, or tissue-specific (e.g., a liver-specific promoter).

The term “enhancer” refers to a nucleotide sequence that can increase the transcription of an operably linked nucleic acid (e.g., a nucleic acid encoding a polypeptide (e.g., any of the exemplary polypeptides described herein). An enhancer can increase the level of transcription by providing additional binding sites for transcription-associated proteins (e.g., transcription factors). Unlike promoters, enhancers can act at distances further away from the transcription start site (e.g., as compared to a promoter).

The terms “identical” or percent “identity,” in the context of two or more polypeptide sequences, refer to two or more sequences or subsequences that are the same or have a specified percentage of amino acid residues, e.g., at least 60%, at least 65%, at least 70%, at least 75%, at least 80%, at least 85%, at least 90%, or at least 95% or greater, that are identical over a specified region when compared and aligned for maximum correspondence over a comparison window or designated region, as measured using a sequence comparison algorithm or by manual alignment and visual inspection.

For sequence comparison of polypeptides, typically one amino acid sequence acts as a reference sequence, to which a candidate sequence is compared. Alignment can be performed using various methods available to one of skill in the art, e.g., visual alignment or using publicly available software using known algorithms to achieve maximal alignment. Such programs include the BLAST programs, ALIGN, ALIGN-2 or Megalign. The parameters employed for an alignment to achieve maximal alignment can be determined by one of skill in the art. For sequence comparison of polypeptide sequences for purposes of this application, the BLASTP algorithm standard protein BLAST for aligning two proteins sequence with the default parameters is used.

The term “affinity” refers to the strength of the sum of all non-covalent interactions between an antigen-binding site and its antigen. Unless otherwise indicated, “affinity” refers to intrinsic binding affinity, which reflects a 1:1 interaction between an antigen-binding domain and an antigen. Affinity can be measured, e.g., using surface plasmon resonance (SPR) technology (e.g., BIACORE®) or biolayer interferometry (e.g., FORTEBIO®). Additional methods for determining the affinity of an antigen-binding domain and its antigen are known in the art.

The term “antigen binding complex” means a complex of two or more (e.g., 2, 3, 4, 5, 6, 7, or 8) polypeptide chains (e.g., the same or different polypeptide chains) that covalently and/or non-covalently associate with each other where at least one of the polypeptide chains binds an antigen. For example, two or more polypeptide chains of a multi-chain polypeptide can associate through the use of two domains that associate with each other (e.g., two Fc domains or IL-15 and the sushi domain of IL-15 receptor alpha).

“Linker” can mean a molecule capable of forming covalent bonds to both TSLP-TRAP and to the moiety. Suitable linkers are well known to those of skill in the art and include, but are not limited to, straight or branched-chain carbon linkers, heterocyclic carbon linkers, or peptide linkers. Where the TSLP-TRAP and the moiety are polypeptides, the linkers may be joined to the constituent amino acids through their side groups (e.g., through a disulfide linkage to cysteine). In some embodiments, the linkers will be joined to the alpha carbon amino and carboxyl groups of the terminal amino acids. Linkers are generally classified into three categories according to their structures: flexible linkers (e.g., (G)n or (GGGGS)n) (SEQ ID NO: 40), rigid linkers (e.g., (EAAAK)n (SEQ ID NO:41)), wherein n is an integer between 1 and 100, and in vivo cleavable linkers (e.g., disulfide or protease sensitive sequences). In some embodiments, a “first” linker refers to a polypeptide between 4 amino acids and 500 amino acids in length that associates the TSLP-TRAP to the moiety. In some embodiments, a “second” linker refers to a different polypeptide between 4 amino acids and 500 amino acids in length that associates the extracellular domain of TSLPR to the extracellular domain of IL-7Rα.

The term “TSLP-TRAP” means a complex of the extracellular domain of thymic stromal lymphopoietin protein receptor (TSLPR) and the extracellular domain of interleukin-7 receptor subunit alpha (IL-7Rα).

The term “TSLP-TRAP complex” refers to a TSLP-TRAP associated with a moiety via a linker.

The section headings used herein are for organizational purposes only and are not to be construed as limiting the subject matter described.

The term “variable domain residue numbering as in Kabat” or “amino acid position numbering as in Kabat”, and variations thereof, refers to the numbering system used for heavy chain variable domains or light chain variable domains of the compilation of antibodies in Kabat et al., Sequences of Proteins of Immunological Interest. 5th Ed. Public Health Service. National Institutes of Health, Bethesda, Md. (1991). Using this numbering system, the actual linear amino acid sequence may contain fewer or additional amino acids corresponding to a shortening of, or insertion into, a FR or CDR of the variable domain. The Kabat numbering of residues may be determined for a given antibody by alignment at regions of homology of the sequence of the antibody with a “standard” Kabat numbered sequence. It will be apparent to those skilled in the art that there are alternative numbering conventions for amino acid residues in variable domain sequences and full-length antibody sequences. There are also alternative numbering conventions for CDR sequences, for example those set out in Chothia et al. (1989) Nature 342:877-883.

Other numbering conventions for CDR sequences available to a skilled person include “AbM” (University of Bath) and “contact” (University College London) methods. The minimum overlapping region using at least two of the Kabat, Chothia, AbM and contact methods can be determined to provide the ‘minimum binding unit’. The minimum binding unit may be a sub-portion of a CDR. Table 1 below represents one definition using each numbering convention for each CDR or binding unit. The Kabat numbering scheme is used in Table 1 to number variable domain amino acid sequence. In should be noted that some of the CDR definitions may vary depending on the individual publication used.

TABLE 1
The numbering scheme

| | Kabat | Chothia | AbM | Contact | Minimum |
|---|---|---|---|---|---|
| | CDR | CDR | CDR | CDR | Binding Unit |
| H1 | 31-35/ | 26-32/ | 26-35/ | 30-35/ | 31-32 |
| | 35A/35B | 33/34 | 35A/35B | 35A/35B | |
| H2 | 50-65 | 52-56 | 50-58 | 47-58 | 52-56 |
| H3 | 95-102 | 95-102 | 95-102 | 93-101 | 95-101 |
| L1 | 24-34 | 24-34 | 24-34 | 30-36 | 30-34 |
| L2 | 50-56 | 50-56 | 50-56 | 46-55 | 50-55 |
| L3 | 89-97 | 89-97 | 89-97 | 89-96 | 89-96 |

### TSLP-TRAP

The TSLP-TRAP of the present disclosure comprises an extracellular domain of thymic stromal lymphopoietin protein receptor (TSLPR) and an extracellular domain of interleukin-7 receptor subunit alpha (IL-7Rα). In some embodiments, the TSLPR is joined to a first portion of the moiety and the IL-7Rα is joined to a second portion of the moiety. In some embodiments, the TSLPR and the IL-7Rα is joined via a linker. In some embodiments, the extracellular domain of TSLPR is derived from a mammal and the extracellular domain of IL-7Rα is derived from a mammal. Mammal refers to any member of the class Mammalia, including, without limitations, humans and nonhuman primates such as chimpanzees and other apes and monkey species; farm animals such as cattle, sheep, pigs, goats, lamas and horses; domestic mammals such as dogs and cats; laboratory animals including rodents such as mice, rats, and guinea pigs, etc.

The TSLP-TRAP may have at least one or more amino acid modifications in various regions of the extracellular domain of TSLPR or the extracellular domain of IL-7Rα. The TSLP-TRAP may be modified by at least one or more amino acid substitution, deletion, addition, or a combination thereof at one or more position while the variant TSLP-TRAP binding complex substantially retains the biological characteristics of the unmodified complex, such as binding to TSLP. In some embodiments, the amino acid sequence of the TSLP-TRAP may have at least 1, at least 2, at least 3, at least 4, at least 5, at least 6, at least 7, at least 8, at least 9, at least 10, at least 11, at least 12, at least 13, at least 14, or at least 15 amino acid insertions, deletions, substitutions or a combination thereof.

In some embodiments, the extracellular domain of TSLPR comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 1. In some embodiments, the extracellular domain of TSLPR comprises an amino acid sequence set out in SEQ ID NO: 1.

In some embodiments the extracellular domain of IL-7Rα comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 2. In some embodiments, the extracellular domain of IL-7Rα comprises an amino acid sequence set out in SEQ ID NO: 2.

The extracellular domain of TSLPR and the extracellular domain of IL-7Rα may be further linked by a linker (second linker). In some embodiments, the TSLP-TRAP may comprise a second linker polypeptide. In some embodiments, the linker may be a flexible linker, a rigid linker, or a cleavable linker. The TSLP-TRAP described herein can be optimized for protein expression and yield by changing composition and/or length of the polypeptide linker. In some embodiments, the second linker is a polypeptide between 3 amino acids and 500 amino acids in length that associates the extracellular domain of TSLPR to the extracellular domain of IL-7Rα. In some embodiments, the polypeptide linker may be about 3 amino acids to about 10 amino acids, about 3 amino acids to about 20 amino acids, about 3 amino acids to about 30 amino acids, about 3 amino acids to about 60 amino acids, about 3 amino acids to about 90 amino acids, about 3 amino acids to about 100 amino acids, about 3 amino acids to about 200 amino acids, about 3 amino acids to about 300 amino acids, about 3 amino acids to about 400 amino acids, about 3 amino acids to about 500 amino acids, about 6 amino acids to about 10) amino acids, about 6 amino acids to about 20 amino acids, about 6 amino acids to about 30 amino acids, about 6 amino acids to about 60 amino acids, about 6 amino acids to about 100 amino acids, about 6 amino acids to about 200 amino acids, about 6 amino acids to about 300 amino acids, about 6 amino acids to about 400 amino acids, about 6 amino acids to about 500 amino acids, about 8 amino acids to about 10) amino acids, about 8 amino acids to about 20 amino acids, about 8 amino acids to about 30 amino acids, about 8 amino acids to about 60 amino acids, about 8 amino acids to about 100 amino acids, about 8 amino acids to about 200 amino acids, about 8 amino acids to about 300 amino acids, about 8 amino acids to about 500 amino acids, about 10 amino acids to about 20 amino acids, about 10) amino acids to about 30 amino acids, about 10) amino acids to about 100 amino acids, about 10 amino acids to about 200 amino acids, about 10 amino acids to about 300 amino acids, about 10 amino acids to about 400 amino acids, about 10 amino acids to about 500 amino acids, about 20 amino acids to about 30 amino acids, about 20 amino acids to about 100 amino acids, about 20 amino acids to about 200 amino acids, about 20 amino acids to about 300 amino acids, about 20 amino acids to about 400 amino acids, about 20 amino acids to about 500 amino acids, about 30 amino acids to about 100 amino acids, about 30 amino acids to about 200 amino acids, about 30 amino acids to about 300 amino acids, about 30) amino acids to about 400 amino acids, about 30 amino acids to about 500 amino acids, about 100 amino acids to about 200 amino acids, about 100 amino acids to about 300 amino acids, about 100 amino acids to about 400 amino acids, about 100 amino acids to about 500 amino acids, about 200 amino acids to about 300 amino acids, about 200 amino acids to about 400 amino acids, about 200 amino acids to about 500 amino acids, about 300 amino acids to about 400) amino acids, about 300 amino acids to about 500 amino acids, or about 400 amino acids to about 500) amino acids. In some embodiments, the polypeptide linker can be about 3 amino acids, about 6 amino acids, about 10 amino acids, about 20 amino acids, about 30) amino acids, about 60) amino acids, about 100 amino acids, about 200 amino acids, about 300 amino acids, about 400) amino acids, or about 500 amino acids. In some embodiments, the polypeptide linker can be at least about 3 amino acids, about 6 amino acids, about 10 amino acids, about 20) amino acids, about 30 amino acids, about 60 amino acids, about 100 amino acids, about 200 amino acids, about 300 amino acids, about 400 amino acids, or about 500 amino acids. In some embodiments, the polypeptide linker may at most be about 6 amino acids, about 10 amino acids, about 20 amino acids, about 30) amino acids, about 100 amino acids, about 200 amino acids, about 300 amino acids, about 400 amino acids, about 500 amino acids.

In some embodiments, the second linker is a polypeptide 60 amino acids in length that associates the extracellular domain of TSLPR to the extracellular domain of IL-7Rα. In some embodiments, the second linker is a polypeptide at least 60 amino acids in length that associates the extracellular domain of TSLPR to the extracellular domain of IL-7Rα. In some embodiments, the second linker is a polypeptide at most 60 amino acids in length that associates the extracellular domain of TSLPR to the extracellular domain of IL-7Rα. For example, in some embodiments. N-terminus of the linker (e.g., the 60) amino acids polypeptide described herein) is coupled to the extracellular domain of the TSLPR, while C-terminus of the linker is coupled to the extracellular domain of IL-7Rα. In some embodiments. C-terminus of the linker (e.g., the 60) amino acids polypeptide described herein) is coupled to the extracellular domain of the TSLPR, while N-terminus of the linker is coupled to the extracellular domain of IL-7Rα.

In some embodiments, the second linker is a polypeptide 20 amino acids in length that associates the extracellular domain of TSLPR to the extracellular domain of IL-7Rα. In some embodiments, the second linker is a polypeptide at least 20 amino acids in length that associates the extracellular domain of TSLPR to the extracellular domain of IL-7Rα. In some embodiments, the second linker is a polypeptide at most 20 amino acids in length that associates the extracellular domain of TSLPR to the extracellular domain of IL-7Rα. For example, in some embodiments. N-terminus of the linker (e.g., the 20 amino acids polypeptide described herein) is coupled to the extracellular domain of the TSLPR, while C-terminus of the linker is coupled to the extracellular domain of IL-7Rα. In some embodiments. C-terminus of the linker (e.g., the 20 amino acids polypeptide described herein) is coupled to the extracellular domain of the TSLPR, while N-terminus of the linker is coupled to the extracellular domain of IL-7Rα. In some embodiments, the second linker is a polypeptide 60 amino acids in length that associates the TSLP-TRAP to the moiety. In some embodiments, the second linker is a polypeptide at least 60 amino acids in length that associates the TSLP-TRAP to the moiety. In some embodiments, the second linker is a polypeptide at most 60 amino acids in length that associates the TSLP-TRAP to the moiety. For example, in some embodiments. N-terminus of the linker (e.g., the 60) amino acids polypeptide described herein) is coupled to the TSLP-TRAP, while C-terminus of the linker is coupled to the moiety. In some embodiments. C-terminus of the linker (e.g., the 60) amino acids polypeptide described herein) is coupled to the extracellular domain of the TSLP-TRAP, while N-terminus of the linker is coupled to the moiety.

In some embodiments, the second linker is a polypeptide 20 amino acids in length that associates the TSLP-TRAP to the moiety. In some embodiments, the second linker is a polypeptide at least 20 amino acids in length that associates the TSLP-TRAP to the moiety. In some embodiments, the second linker is a polypeptide up to and including 20 amino acids in length that associates the TSLP-TRAP to the moiety. For example, N-terminus of the linker (e.g., the 20 amino acids polypeptide described herein) is coupled to the TSLP-TRAP, while C-terminus of the linker is coupled to the moiety. In some embodiments. C-terminus of the linker (e.g., the 20 amino acids polypeptide described herein) is coupled to the extracellular domain of the TSLP-TRAP, while N-terminus of the linker is coupled to the moiety.

In some embodiments, the polypeptide linker described herein may be about 15 amino acids in length, about 20 amino acids in length, about 25 amino acids in length, about 30 amino acids in length, about 35 amino acids in length, about 40 amino acids in length, about 45 amino acids in length, about 50 amino acids in length, about 55 amino acids in length, or about 60 amino acids in length. In some embodiments, the linker peptide may comprise (GGS)n·(GGGGS)n (SEQ ID NO: 40), (SGGGG)n (SEQ ID NO: 42), GGGG(SGGGG)n (SEQ ID NO: 43) or GG(SGG)n (SEQ ID NO:44) wherein n is an integer between 1 and 100. In some embodiments, the linker peptide may comprise (GGS)x20 (SEQ ID NO: 3).

The TSLP-TRAP can be arranged as either 1) the extracellular domain of TSLPR-second linker—the extracellular domain of IL-7Rα or 2) the extracellular domain of IL-7Rα-second linker—the extracellular domain of TSLPR. In some embodiments, the TSLP-TRAP comprises amino acids 25-231 of human TSLPR associated via (GGS)x20 (SEQ ID NO: 3) to amino acids 21-239 of human IL-7Rα. In some embodiments, the TSLP-TRAP comprises amino acids 21-239 of human IL-7Rα associated via (GGS)x20 (SEQ ID NO: 3) to amino acids 25-231 of human TSLPR. In some embodiments, the TSLP-TRAP comprises at least about 15, 20, 25, 30 or 50 contiguous amino acids of human TSLPR associated via a linker to at least about 15, 20, 25, 30 or 50 contiguous amino acids of human IL-7Rα.

In some embodiments, the TSLP-TRAP comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 4. In some embodiments, the TSLP-TRAP comprises an amino acid sequence set out in SEQ ID NO: 4.

In some embodiments, the TSLP-TRAP comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 5. In some embodiments, the TSLP-TRAP comprises an amino acid sequence set out in SEQ ID NO: 5.

In some embodiments, the antigen binding complex comprising the TSLP-TRAP associated with a moiety via a linker as described herein may be administered to a subject for diagnosis or treatment of a condition or a disease. Exemplary conditions and diseases may include, but not limited to, asthma, inflammatory arthritis, sinusitis, pruritus, solid tumors, atopic dermatitis, eczema, eosinophilic esophagitis, allergic rhinitis, airway hyperresponsiveness, airway inflammation, a food allergy, chronic urticaria, occupational allergy, allergic conjunctivitis, hay fever, airborne allergic sensitivities, stinging insect allergy, hypersensitivity pneumonitis, eosinophilic lung diseases, inflammatory bowel disease, ulcerative colitis, Crohn's disease and drug allergies.

### First Linker

In some embodiments, the first linker is capable of forming covalent bonds to both TSLP-TRAP and to the moiety. Suitable linkers are well known to those of skill in the art and include, but are not limited to, straight or branched-chain carbon linkers, heterocyclic carbon linkers, or peptide linkers. Where the TSLP-TRAP and the moiety are polypeptides, the linkers may be joined to the constituent amino acids through their side groups (e.g., through a disulfide linkage to cysteine). In some embodiments, the linkers can be joined to the alpha carbon amino and carboxyl groups of the terminal amino acids.

The TSLP-TRAP and a moiety may be further associated by a linker (first linker). In some embodiments, the TSLP-TRAP complex may comprise a first linker polypeptide. In some embodiments, the linker may be a flexible linker, a rigid linker, or a cleavable linker. The TSLP-TRAP complex described herein can be optimized for protein expression and yield by changing composition and/or length of the polypeptide linker. In some embodiments, the first linker is a polypeptide between 3 amino acids and 500 amino acids in length that associates the TSLP-TRAP to the moiety. In some embodiments, the polypeptide linker may be about 3 amino acids to about 10) amino acids, about 3 amino acids to about 20 amino acids, about 3 amino acids to about 30) amino acids, about 3 amino acids to about 60) amino acids, about 3 amino acids to about 90 amino acids, about 3 amino acids to about 100 amino acids, about 3 amino acids to about 200 amino acids, about 3 amino acids to about 300 amino acids, about 3 amino acids to about 400 amino acids, about 3 amino acids to about 500 amino acids, about 6 amino acids to about 10) amino acids, about 6 amino acids to about 20 amino acids, about 6 amino acids to about 30) amino acids, about 6 amino acids to about 60) amino acids, about 6 amino acids to about 100 amino acids, about 6 amino acids to about 200 amino acids, about 6 amino acids to about 300 amino acids, about 6 amino acids to about 400 amino acids, about 6 amino acids to about 500) amino acids, about 8 amino acids to about 10 amino acids, about 8 amino acids to about 20 amino acids, about 8 amino acids to about 30 amino acids, about 8 amino acids to about 60) amino acids, about 8 amino acids to about 100 amino acids, about 8 amino acids to about 200 amino acids, about 8 amino acids to about 300 amino acids, about 8 amino acids to about 500 amino acids, about 10 amino acids to about 20 amino acids, about 10 amino acids to about 30) amino acids, about 10) amino acids to about 100 amino acids, about 10 amino acids to about 200 amino acids, about 10) amino acids to about 300 amino acids, about 10 amino acids to about 400) amino acids, about 10) amino acids to about 500 amino acids, about 20 amino acids to about 30) amino acids, about 20 amino acids to about 100 amino acids, about 20 amino acids to about 200 amino acids, about 20 amino acids to about 300 amino acids, about 20 amino acids to about 400 amino acids, about 20 amino acids to about 500 amino acids, about 30 amino acids to about 100 amino acids, about 30 amino acids to about 200 amino acids, about 30 amino acids to about 300 amino acids, about 30 amino acids to about 400 amino acids, about 30 amino acids to about 500) amino acids, about 100 amino acids to about 200 amino acids, about 100 amino acids to about 300 amino acids, about 100 amino acids to about 400 amino acids, about 100 amino acids to about 500 amino acids, about 200 amino acids to about 300 amino acids, about 200 amino acids to about 400 amino acids, about 200 amino acids to about 500 amino acids, about 300 amino acids to about 400 amino acids, about 300 amino acids to about 500 amino acids, or about 400 amino acids to about 500 amino acids. In some embodiments, the polypeptide linker can be about 3 amino acids, about 6 amino acids, about 10 amino acids, about 20 amino acids, about 30) amino acids, about 60) amino acids, about 100 amino acids, about 200 amino acids, about 300 amino acids, about 400 amino acids, or about 500 amino acids. In some embodiments, the polypeptide linker can be at least about 3 amino acids, about 6 amino acids, about 10) amino acids, about 20 amino acids, about 30 amino acids, about 60) amino acids, about 100) amino acids, about 200 amino acids, about 300 amino acids, about 400 amino acids, or about 500) amino acids. In some embodiments, the polypeptide linker may at most be about 6 amino acids, about 10) amino acids, about 20 amino acids, about 30) amino acids, about 100 amino acids, about 200 amino acids, about 300) amino acids, about 400 amino acids, about 500 amino acids.

In some embodiments, the first linker is a polypeptide 60 amino acids in length that associates the TSLP-TRAP to the moiety. In some embodiments, the first linker is a polypeptide at least 60 amino acids in length that associates the TSLP-TRAP to the moiety. In some embodiments, the first linker is a polypeptide up to and including 60 amino acids in length that associates the TSLP-TRAP to the moiety. In some embodiments, the first linker is a polypeptide 20) amino acids in length that associates the TSLP-TRAP to the moiety. In some embodiments, the first linker is a polypeptide at least 20 amino acids in length that associates the TSLP-TRAP to the moiety. In some embodiments, the first linker is a polypeptide up to and including 20 amino acids in length that associates the TSLP-TRAP to the moiety. In some embodiments, the polypeptide linker may be about 15 amino acids in length, about 20 amino acids in length, about 25 amino acids in length, about 30 amino acids in length, about 35 amino acids in length, about 40) amino acids in length, about 45 amino acids in length, about 50 amino acids in length, about 55 amino acids in length, or about 60 amino acids in length.

In some embodiments, the linker peptide may comprise (GGS)n, (GGGGS)n (SEQ ID NO: 40), (SGGGG)n (SEQ ID NO: 42), GGGG(SGGGG)n (SEQ ID NO: 43), or GG(SGG)n (SEQ ID NO: 44) wherein n is an integer between 1 and 100. In some embodiments, the linker peptide may comprise (GGGGS)x4 (SEQ ID NO: 6).

In some embodiments, the first linker comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence of (GGGGS)n (SEQ ID NO: 40), wherein n is an integer between 1 and 100. In some embodiments, the first linker comprises an amino acid sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence of (GGGGS)x4 (SEQ ID NO: 6). In some embodiments, the amino acid sequence of the first linker comprises (GGGGS)x4 (SEQ ID NO: 6).

### Moieties

In some embodiments, the antigen binding complex comprises a moiety selected from the group consisting of: 1) a probe; 2) an antibody domain; 3) an antibody or antigen binding portion thereof; 4) a receptor; and 5) a half-life extender.

In some embodiments, the moiety is a probe, wherein the probe provides additional means of isolating and/or detecting the translated antigen binding complex. Suitable probes (also known as labels or tags) are well known in the art and, for example, include, but are not limited to luciferase, green fluorescent protein, alkaline phosphatase, horseradish peroxidase, myc-tags, FLAG tags, eTags and polyhistidine tags. In some embodiments, such labels and tags are capable of providing a detectable signal to facilitate identification of such labels or tags, for example upon distribution of the amino acid sequence encoding the antigen binding complex into the desired cells and tissues. In some embodiments, the moiety is the probe, wherein the probe may be a His tag, a GST tag, a flag tag, or a fluorescent molecule.

In some embodiments, the moiety comprises an antibody domain, wherein the antibody domain comprises a Fc region. In some embodiments, the Fc region is an IgG1, IgG2, IgG3, or IgG4 heavy chain Fc region. In some embodiments, the Fc region comprises M252Y/S254T/T256E (YTE), M428L/N434S (LS), T307A/E380A/N434A (AAA), T250Q/M428L (QL), or V308P sequence mutations.

In some embodiments, the moiety is the antibody or antigen binding portion thereof. In some embodiments, the antibody or antigen binding portion thereof described herein may be a monoclonal antibody, a polyclonal antibody, a bispecific antibody, a multispecific antibody, a grafted antibody, a human antibody, a humanized antibody, a synthetic antibody, a chimeric antibody, a camelized antibody, a single-chain Fvs (scFv), a single chain antibody, a Fab fragment, a F(ab′)2 fragment, a Fd fragment, a Fv fragment, a single-domain antibody, a diabody, a fragment comprised of only a single monomeric variable domain, disulfide-linked Fvs (sdFv), an intrabody, an anti-idiotypic (anti-Id) antibody, a VHH antibody, or ab antigen-binding fragment thereof.

The antibody or antigen-binding fragment thereof may have at least one or more amino acid modifications in various regions of the antibody. The antibody or antigen-binding fragment thereof may be modified by at least one or more amino acid substitution, deletion, addition, or a combination thereof at one or more position while the variant binding complex substantially retains the biological characteristics of the unmodified protein, such as binding to its target. In some embodiments, the amino acid sequence of the antibody or antigen-binding fragment thereof may have at least 1, at least 2, at least 3, at least 4, at least 5, at least 6, at least 7, at least 8, at least 9, at least 10, at least 11, at least 12, at least 13, at least 14, or at least 15 amino acid insertions, deletions, substitutions or a combination thereof.

In some instances, one or more amino acid modifications described herein can be in the antigen-binding (Fab) fragments of an antibody or antibody binding fragment thereof. In some instances, the modification can be within the Fab region of a heavy chain polypeptide (H) and a first light chain polypeptide (L). In some embodiments, the modification can be within a heavy chain variable domain (VH), a heavy chain constant domain (CH1), a light chain variable domain (VL), a light chain constant domain (CL), or a combination thereof. In some embodiments, one or more amino acid modification can be in the Fab VH and VL with the N-terminal region. The VH, VL, HC or LC sequence disclosed herein may be a variant sequence with up to 15 amino acid substitutions, insertion, or deletions. For example, the variant sequence may have up to 15, 14, 13, 12, 11, 10, 9, 8, 7, 6, 5, 4, 3, 2, or 1 amino acid substitution(s), addition(s), or deletion(s). The sequence variation may exclude one or more or all of the CDRs, for example, the CDRs are the same as the VH, VL, HC or LC sequence and the variation is in the remaining portion of the VH or VL, HC, or LC sequence, so that the CDR sequences are fixed and intact.

Each of HCDR1, HCDR2, HCDR3, LCDR1, LCDR2, and LCDR3 may be modified alone or in combination with any other CDR, in any permutation or combination. Typically, the modification is a substitution, particularly a conserved substitution, for example in Table 2 below.

TABLE 2
List of side chains & members

| Side Chain | Members |
|---|---|
| Hydrophobic | Met, Ala, Val, Leu Ile |
| Neutral Hydrophilic | Cys, Ser, Thr |
| Acidic | Asp, Glu |
| Basic | Asn, Gln, His, Lys, Arg |
| Residues that influence chain orientation | Gly, Pro |
| Aromatic | Trp, Tyr, Phe |

The antibodies of use can be of various isotypes, preferably human IgG1, IgG2, IgG3 or IgG4, more preferably comprising human IgG1 hinge and constant region sequences. The antibodies or fragments thereof can be chimeric human-mouse, humanized (human framework and murine hypervariable (CDR) regions), or fully human, as well as variations thereof, such as half-IgG4 antibodies (referred to as “unibodies. More preferably, the antibodies or fragments thereof may be designed or selected to comprise human constant region sequences that belong to specific allotypes, which may result in reduced immunogenicity when administered to a human subject. In some embodiments, allotypes for administration include a non-Glm1 allotype (nGlm1), such as Glm3, Glm3,1, Glm3,2 or Glm3,1,2. More preferably, the allotype is selected from the group consisting of the nGlm1, Glm3, nGlm1,2 and Km3 allotypes.

In some embodiments, the antibody or antigen binding portion thereof specifically binds to CCL19, CCL21, CD1, CD1a, CD2, CD3, CD4, CD5, CD8, CD11A, CD14, CD15, CD16, CD17, CD18, CD19, CD20, CD21, CD22, CD23, CD25, CD29, CD30, CD32b, CD33, CD37, CD38, CD40, CD40L, CD44, CD45, CD46, CD52, CD54, CD55, CD59, CD64, CD66a-e, CD67, CD70, CD70L, CD74, CD79a, CD80, CD83, CD95, CD126, CD132, CD133, CD138, CD147, CD154, CDC27, CDK-4/m, CDKN2A, CTLA-4, CXCR4, CXCR7, CXCL12, CDH1, CDH6, CDH17, CDH19, CDH179b, HIF-1a, colon-specific antigen-p (CSAp), CEA (CEACAM5), CEACAM6, c-Met, DAM, EGFR, EGFRvIII, EGP-1 (TROP-2), EGP-2, ELF2-M, Ep-CAM, fibroblast growth factor (FGF), Flt-1, Flt-3, folate receptor, G250 antigen, GAGE, gp100, GRO-β, HLA-DR, HM1.24, human chorionic gonadotropin (HCG) and its subunits, HER2/neu, HMGB-1, hypoxia inducible factor (HIF-1), HSP70-2M, HST-2, Ia, IGF-1R, IFN-γ, IFN-α, IFN-β, IFN-2, IL-4R, IL-6R, IL-13R, IL-15R, IL-17R, IL-18R, IL-2, IL-4, IL-5, IL-6, IL-8, IL-12, IL-15, IL-17, IL-18, IL-23, IL-25, IL-31, IL-33, CC12, CCL3, CCL4, CCL5, CCL6, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27 CCL28, CXCL1, CXCL2, CXCL3, CXCL4, CXCL5, CXCL6, CXCL7, CXCL9, CXCL10, CXCL11, CXCL12, CXCL13, CXCL16, insulin-like growth factor-1 (IGF-1), KC4-antigen, KS-1-antigen, KS1-4, Le-Y, LDR/FUT, macrophage migration inhibitory factor (MIF), MAGE, MAGE-3, MART-1, MART-2, NY-ESO-1, TRAG-3, mCRP, MCP-1, MIP-1A, MIP-1B, MIF, MUC1, MUC2, MUC3, MUC4, MUC5ac, MUC13, MUC16, MUM-1/2, MUM-3, NCA66, NCA95, NCA90, pancreatic cancer mucin, PD-1 receptor, placental growth factor, p53, PLAGL2, prostatic acid phosphatase, PSA, PRAME, PSMA, P1GF, ILGF, RS5, RANTES, T101, SAGE, S100, survivin, survivin-2B, TAC, TAG-72, tenascin, TRAIL receptors, TNF-α, CCR1, CCR2, CCR3, CCR4, CCR5, CCR6, CCR7, CCR8, CCR9, CCR10, CCR11, CXCR1, CXCR2, CXCR3, CXCR4, CXCR5 or CXCR6. In some embodiments, the antibody or antigen binding portion thereof specifically binds human interleukin-4 receptor (hIL-4R), human interleukin-4 (hIL-4), human interleukin-13 receptor (hIL-13R), human interleukin-13 (hIL-13), human interleukin-5 (hIL-5), human interleukin-5 receptor (hIL-5R), human interleukin-33 (hIL-33) or human interleukin-31 (hIL-31).

In some embodiments, the TSLP-TRAP is associated with a C terminus or termini of the moiety. In some embodiments, the TSLP-TRAP is associated with a N terminus or termini of the moiety.

In some embodiments, the moiety is a receptor. In some embodiments, the receptor can be CD1, CD1a, CD2, CD3, CD4, CD5, CD8, CD11A, CD14, CD15, CD16, CD17, CD18, CD19, CD20, CD21, CD22, CD23, CD25, CD29, CD30, CD32b, CD33, CD37, CD38, CD40, CD44, CD45, CD46, CD52, CD54, CD55, CD59, CD64, CD66a-e, CD67, CD70, CD74, CD79a, CD80, CD83, CD95, CD126, CD132, CD133, CD138, CD147, CD154, CDC27, CDK-4/m, CDKN2A, CTLA-4, CXCR4, CXCR7, CXCL12, CDH1, CDH6, CDH17, CDH19, CDH179b, EGFR, EGFRvIII, folate receptor, HLA-DR, HER2/neu, HMGB-1, IGF-1R, IL-4R, IL-6R, IL-13R, IL-15R, IL-17R, IL-18R, TRAIL receptors, TNFR1, TNFR2, PDG2R, CRTH2, CCR1, CCR2, CCR3, CCR4, CCR5, CCR6, CCR7, CCR8, CCR9, CCR10, CCR11, CXCR1, CXCR2, CXCR3, CXCR4, CXCR5 or CXCR6.

In some embodiments, the antibody or antigen binding portion thereof is or comprise dupilumab, pascolizumab, CM-310, recombinant anti-IL-4Rα humanized monoclonal antibody. MG-K10, manfidokimab, QX-005N, elarekibep, CBP-201, SHR-1819, LQ-036, BA2101, mepolizumab, reslizumab, benralizumab, GSK3511294, AK-120, pitrakinra, anrukinzumab, IMA-638, lebrikizumab, tralokinumab, GSK679586, AMG-317, MILR1444A, CAT-354, QAX576, IMA-026, CNTO-607, MK-6105, DOM-0910, SAR440340, REGN3500, etokimab, ANB020, astegolimab, tozorakimab, MEDI3506, CNTO7160), torudokimab, itepekimab, dupixent, nemolizumab, BMS-981164, lokivetmab, vixarelimab or an antigen binding portion thereof.

In some embodiments the moiety is a half-life extender. In some embodiments, the half-life extender comprises an antibody or antigen binding portion that is modified to extend the complex's half-life in blood. In some embodiments, the half-life extender is albumin, PEG, or a suitable polymer to extend half-life in blood.

### Anti-IL-4R TSLP-TRAP Complexes

In some embodiments, the TSLP-TRAP complex comprises a TSLP-TRAP associated to an antibody or antigen-binding portion thereof that specifically binds human interleukin-4 receptor (hIL-4R). In some embodiments, the antibody or antigen-binding portion thereof specifically binds human interleukin-4 receptor (hIL-4R). In some embodiments, the antibody or antigen binding portion thereof comprises a HCDR1 having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 7, (b) a HCDR2 having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 8, and/or (c) a HCDR3 having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 9. In some embodiments, the antibody or antigen binding portion thereof comprises (a) a HCDR1 comprising an amino acid sequence of SEQ ID NO: 7, (b) a HCDR2 comprising an amino acid sequence of SEQ ID NO: 8, and/or (c) a HCDR3 comprising an amino acid sequence set out in SEQ ID NO: 9.

In some embodiments, the antibody or antigen binding portion thereof comprises a heavy chain variable domain having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 10. In some embodiments, the antibody or antigen binding portion thereof comprises a heavy chain variable domain comprising an amino acid sequence out in SEQ ID NO: 10. For example, in some embodiments, the antibody or antigen binding portion thereof comprises a heavy chain variable domain (e.g., SEQ ID NO: 10) coupled to one or more TSLP-TRAP complex disclosed herein via one or more linker.

In some embodiments, the antibody or antigen binding portion thereof comprises a heavy chain having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 11. In some embodiments, the antibody or antigen binding portion thereof comprises a heavy chain variable domain comprising an amino acid sequence out in SEQ ID NO: 11. For example, in some embodiments, the antibody or antigen binding portion thereof comprises a heavy chain (e.g., SEQ ID NO: 11) coupled to one or more TSLP-TRAP complex disclosed herein via one or more linker.

In some embodiments, the antibody or antigen binding portion thereof comprises a heavy chain Fc region comprising M252Y/S254T/T256E (YTE), M428L/N434S (LS), T307A/E380A/N434A (AAA), T250Q/M428L (QL), or V308P sequence mutations. In some embodiments, the antibody or antigen binding portion thereof comprises human IgG4 isotype with a S228P stabilized hinge.

In some embodiments, the antibody or antigen binding portion thereof comprises (a) a LCDR1 having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 12, (b) a LCDR2 having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 13, and/or (c) a LCDR3 having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 14. In some embodiments, the antibody or antigen binding portion thereof comprises (a) a LCDR1 comprising an amino acid sequence of SEQ ID NO: 12, (b) a LCDR2 comprising an amino acid sequence of SEQ ID NO: 13, and/or (c) a LCDR3 comprising an amino acid sequence of SEQ ID NO: 14.

In some embodiments, the antibody or antigen binding portion thereof comprises a light chain variable domain having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 15. In some embodiments, the antibody or antigen binding portion thereof comprises a light chain variable domain comprising an amino acid sequence set out in SEQ ID NO: 15. For example, in some embodiments, the antibody or antigen binding portion thereof comprises a light chain variable domain (e.g., SEQ ID NO: 15) coupled to one or more TSLP-TRAP complex disclosed herein via one or more linker.

In some embodiments, the antibody or antigen binding portion thereof comprises a light chain having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 16. In some embodiments, the antibody or antigen binding portion thereof comprises a light chain comprising an amino acid sequence set out in SEQ ID NO: 16. For example, In some embodiments, the antibody or antigen binding portion thereof comprises a light chain (e.g., SEQ ID NO: 16) coupled to one or more TSLP-TRAP complex disclosed herein via one or more linker.

In some embodiments, the antibody or antigen-binding portion thereof is dupilumab.

In some embodiments, the antibody or antigen binding portion thereof further comprises a Fc region with one or more mutations to extend half-life. In some embodiments, the one or more mutations to extend half-life comprises M252Y/S254T/T256E (YTE), M428L/N434S (LS), T307A/E380A/N434A (AAA), T250Q/M428L (QL), or V308P sequence mutations. In some embodiments, the antibody or antigen binding portion thereof comprises human IgG4 isotype with a S228P stabilized hinge.

### Monospecific TSLP-TRAP Complexes

In some embodiments, the TSLP-TRAP complex comprises a TSLP-TRAP associated with a modified antibody or antigen binding portion thereof that does not bind to an antigen. In some embodiments, the antibody or antigen binding portion thereof does not bind to anything. In some embodiments, the antibody or antigen binding portion thereof does not bind to an endogenous antigen.

In some embodiments, wherein the antibody or antigen binding portion thereof comprises (a) a HCDR1 having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 17, (b) a HCDR2 having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 18, and/or (c) a HCDR3 having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 19. In some embodiments, the antibody or antigen binding portion thereof comprises (a) a HCDR1 comprising an amino acid sequence of SEQ ID NO: 17, (b) a HCDR2 comprising an amino acid sequence of SEQ ID NO: 18, and/or (c) a HCDR3 comprising an amino acid sequence set out in SEQ ID NO: 19.

In some embodiments, the antibody or antigen binding portion thereof comprises a heavy chain variable domain having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 20. In some embodiments, the antibody or antigen binding portion thereof comprises a heavy chain variable domain comprising an amino acid sequence out in SEQ ID NO: 20. For example, in some embodiments, the antibody or antigen binding portion thereof comprises a heavy chain variable domain (e.g., SEQ ID NO: 20) coupled to one or more TSLP-TRAP complex disclosed herein via one or more linker.

In some embodiments, the antibody or antigen binding portion thereof comprises a heavy chain having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 21. In some embodiments, the antibody or antigen binding portion thereof comprises a heavy chain comprising an amino acid sequence set out in SEQ ID NO: 21. For example, in some embodiments, the antibody or antigen binding portion thereof comprises a heavy chain (e.g., SEQ ID NO: 21) coupled to one or more TSLP-TRAP complex disclosed herein via one or more linker.

In some embodiments, the antibody or antigen binding portion thereof comprises a heavy chain Fc region comprising M252Y/S254T/T256E (YTE), M428L/N434S (LS), T307A/E380A/N434A (AAA), T250Q/M428L (QL), or V308P sequence mutations. In some embodiments, the antibody or antigen binding portion thereof comprises human IgG4 isotype with a S228P stabilized hinge.

In some embodiments, the antibody or antigen binding portion thereof comprises (a) a LCDR1 having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 22, (b) a LCDR2 having the amino acid sequence set out in SEQ ID NO: 23, and/or (c) a LCDR3 having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 24. In some embodiments, the antibody or antigen binding portion thereof comprises (a) a LCDR1 comprising an amino acid sequence of SEQ ID NO: 22, (b) a LCDR2 comprising an amino acid sequence of SEQ ID NO: 23, and/or (c) a LCDR3 comprising an amino acid sequence of SEQ ID NO: 24.

In some embodiments, the antibody or antigen binding portion thereof comprises a light chain variable domain having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 25. In some embodiments, the antibody or antigen binding portion thereof comprises a light chain variable domain comprising an amino acid sequence set out in SEQ ID NO: 25. For example, in some embodiments, the antibody or antigen binding portion thereof comprises a light chain variable domain (e.g., SEQ ID NO: 25) coupled to one or more TSLP-TRAP complex disclosed herein via one or more linkers.

In some embodiments, the antibody or antigen binding portion thereof comprises a light chain having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to the amino acid sequence set out in SEQ ID NO: 26. In some embodiments, the antibody or antigen binding portion thereof comprises a light chain comprising an amino acid sequence set out in SEQ ID NO: 26. For example, in some embodiments, the antibody or antigen binding portion thereof comprises a light chain variable domain (e.g., SEQ ID NO: 26) coupled to one or more TSLP-TRAP complex disclosed herein via one or more linker.

In some embodiments, the antibody or antigen-binding portion thereof is palivizumab.

In some embodiments, the antibody or antigen binding portion thereof further comprises a Fc region with one or more mutations to extend half-life. In some embodiments, the one or more mutations to extend half-life comprises M252Y/S254T/T256E (YTE), M428L/N434S (LS), T307A/E380A/N434A (AAA), T250Q/M428L (QL), or V308P sequence mutations. In some embodiments, the antibody or antigen binding portion thereof comprises human IgG4 isotype with a S228P stabilized hinge.

### Multiple TSLP-TRAPS

The TSLP-TRAP complexes may comprise multiple TSLP-TRAPs. In some embodiments, the antigen binding complex comprises at least two TSLP-TRAPs. In some embodiments, the at least two TSLP-TRAP is each separately associated with the moiety via a linker disclosed herein. In some embodiments, a TSLP-TRAP is associated with a second TSLP-TRAP. In some embodiments, the at least two TSLP-TRAP comprises 3 TSLP-TRAPs. In some embodiments, the at least two TSLP-TRAP comprises 4 TSLP-TRAPs. In some embodiments, 1 TSLP-TRAP is linked to the C termini of the antibody or antigen binding fragment thereof light chain. In some embodiments, 1 TSLP-TRAP is linked to the N termini of the antibody or antigen binding fragment thereof light chain. In some embodiments, 1 TSLP-TRAP is linked to the N and C termini of the antibody light chain. In some embodiments, 1 TSLP-TRAP is linked to the C termini of the antibody or antigen binding fragment heavy chain. In some embodiments, 1 TSLP-TRAP is linked to the N termini of the antibody or antigen binding fragment heavy chain. In some embodiments, 1 TSLP-TRAP is linked to the C and N termini of the antibody or antigen binding fragment heavy chain. In some embodiments, 1 TSLP-TRAP is linked to the C and N termini of the antibody or antigen binding fragment heavy and light chains.

### Candidates

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 27. In some embodiments, the antigen binding complex comprises an amino acid sequence of SEQ ID NO: 27.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 28. In some embodiments, the antigen binding complex comprises an amino acid sequence of SEQ ID NO: 28.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 29. In some embodiments, the antigen binding complex comprises an amino acid sequence of SEQ ID NO: 29.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 30. In some embodiments, the antigen binding complex comprises an amino acid sequence of SEQ ID NO: 30.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 31. In some embodiments, the antigen binding complex comprises an amino acid sequence set out in SEQ ID NO: 31.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 32. In some embodiments, the antigen binding complex comprises an amino acid sequence of SEQ ID NO: 32.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 33. In some embodiments, antigen the binding complex comprises an amino acid sequence of SEQ ID NO: 33.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 34. In some embodiments, the antigen binding complex comprises an amino acid sequence of SEQ ID NO: 34.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 35. In some embodiments, the antigen binding complex comprises an amino acid sequence of SEQ ID NO: 35.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 36. In some embodiments, the antigen binding complex comprises an amino acid sequence of SEQ ID NO: 36.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 37. In some embodiments, the antigen binding complex comprises an amino acid sequence of SEQ ID NO: 37.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 38. In some embodiments, the antigen binding complex comprises an amino acid sequence of SEQ ID NO: 38.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 39. In some embodiments, the antigen binding complex comprises an amino acid sequence of SEQ ID NO: 39.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 45. In some embodiments, the antigen binding complex comprises an amino acid sequence of SEQ ID NO: 45.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 46. In some embodiments, the antigen binding complex comprises an amino acid sequence of SEQ ID NO: 46.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 47. In some embodiments, the antigen binding complex comprises an amino acid sequence of SEQ ID NO: 47.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 48. In some embodiments, the antigen binding complex comprises an amino acid sequence of SEQ ID NO: 48.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 49. In some embodiments, the antigen binding complex comprises an amino acid sequence of SEQ ID NO: 49.

In some embodiments, the antigen binding complex comprises a sequence having at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to an amino acid sequence set out in SEQ ID NO: 50. In some embodiments, the antigen binding complex comprises an amino acid sequence of SEQ ID NO: 50.

### Binding Affinity

In some embodiments, the TSLP-TRAP complex has a KD for binding to TSLP of about 0.2×10⁻¹² to 30.0×10⁻¹² M. In some embodiments, TSLP-TRAP complex has a KD for binding to TSLP of about 1.0×10⁻¹² M, about 2.0×10⁻¹² M, about 3.0×10⁻¹² M, about 4.0×10⁻¹² M, about 5.0×10⁻¹² M, about 6.0×10⁻¹² M, about 7.0×10⁻¹² M, about 8.0×10⁻¹² M, about 9.0×10⁻¹² M, about 10.0×10⁻¹² M, about 11.0×10⁻¹² M, about 12.0×10⁻¹² M, about 13.0×10⁻¹² M, about 14.0×10⁻¹² M, about 15.0×10⁻¹² M, about 16.0×10⁻¹² M, about 17.0×10⁻¹² M, about 18.0×10⁻¹² M, about 19.0×10⁻¹² M, about 20.0×10⁻¹² M, about 21.0×10⁻¹² M, about 22.0×10⁻¹² M, about 23.0×10⁻¹² M, about 24.0×10⁻¹² M, about 25.0×10⁻¹² M, about 26.0×10⁻¹² M, about 27.0×10⁻¹² M, about 28.0×10⁻¹²M, about 29.0×10⁻¹² M, about 30.0×10⁻¹² M, about 31.0×10⁻¹² M, about 32.0×10⁻¹² M, about 33.0×10⁻¹² M, about 34.0×10⁻¹² M, about 35.0×10⁻¹² M, about 36.0×10⁻¹² M, about 37.0×10⁻¹² M, about 38.0×10⁻¹² M, about 39.0×10⁻¹² M, about 40.0×10⁻¹² M, about 41.0×10⁻¹² M, about 42.0×10⁻¹² M, about 43.0×10⁻¹² M, about 44.0×10⁻¹² M, about 45.0×10⁻¹² M, about 46.0×10⁻¹² M, about 47.0×10⁻¹² M, about 48.0×10⁻¹² M, about 49.0×10⁻¹² M, or about 50.0×10⁻¹² M. In some embodiments, the TSLP-TRAP complex has a KD for binding to TSLP of less than 1.0×10⁻¹² M, about 2.0×10⁻¹² M, about 3.0×10⁻¹² M, about 4.0×10⁻¹² M, about 5.0×10⁻¹² M, about 6.0×10⁻¹² M, about 7.0×10⁻¹² M, about 8.0×10⁻¹² M, about 9.0×10⁻¹² M, about 10.0×10⁻¹² M, about 11.0×10⁻¹² M, about 12.0×10⁻¹² M, about 13.0×10⁻¹² M, about 14.0×10⁻¹² M, about 15.0×10⁻¹² M, about 16.0×10⁻¹² M, about 17.0×10⁻¹² M, about 18.0×10⁻¹² M, about 19.0×10⁻¹² M, about 20.0×10⁻¹² M, about 21.0×10⁻¹² M, about 22.0×10⁻¹² M, about 23.0×10⁻¹² M, about 24.0×10⁻¹² M, about 25.0×10⁻¹² M, about 26.0×10⁻¹² M, about 27.0×10⁻¹² M, about 28.0×10⁻¹² M, about 29.0×10⁻¹² M, about 30.0×10⁻¹² M, about 31.0×10⁻¹² M, about 32.0×10⁻¹² M, about 33.0×10⁻¹² M, about 34.0×10⁻¹² M, about 35.0×10⁻¹² M, about 36.0×10⁻¹² M, about 37.0×10⁻¹² M, about 38.0×10⁻¹² M, about 39.0×10⁻¹² M, about 40.0×10⁻¹² M, about 41.0×10⁻¹² M, about 42.0×10⁻¹² M, about 43.0×10⁻¹² M, about 44.0×10⁻¹² M, about 45.0×10⁻¹² M, about 46.0×10⁻¹² M, about 47.0×10⁻¹² M, about 48.0×10⁻¹² M, about 49.0×10⁻¹² M, or about 50.0×10⁻¹² M. In some embodiments, the TSLP-TRAP complex has a KD for binding to TSLP of at most 1.0×10⁻¹² M, about 2.0×10⁻¹² M, about 3.0×10⁻¹² M, about 4.0×10⁻¹² M, about 5.0×10⁻¹² M, about 6.0×10⁻¹² M, about 7.0×10⁻¹² M, about 8.0×10⁻¹² M, about 9.0×10⁻¹² M, about 10.0×10⁻¹² M, about 11.0×10⁻¹² M, about 12.0×10⁻¹² M, about 13.0×10⁻¹² M, about 14.0×10⁻¹² M, about 15.0×10⁻¹² M, about 16.0×10⁻¹² M, about 17.0×10⁻¹² M, about 18.0×10⁻¹² M, about 19.0×10⁻¹² M, about 20.0×10⁻¹² M, about 21.0×10⁻¹² M, about 22.0×10⁻¹² M, about 23.0×10⁻¹² M, about 24.0×10⁻¹² M, about 25.0×10⁻¹² M, about 26.0×10⁻¹² M, about 27.0×10⁻¹² M, about 28.0×10⁻¹² M, about 29.0×10⁻¹² M, about 30.0×10⁻¹² M, about 31.0×10⁻¹² M, about 32.0×10⁻¹² M, about 33.0×10⁻¹² M, about 34.0×10⁻¹² M, about 35.0×10⁻¹² M, about 36.0×10⁻¹² M, about 37.0×10⁻¹² M, about 38.0×10⁻¹² M, about 39.0×10⁻¹² M, about 40.0×10⁻¹² M, about 41.0×10⁻¹² M, about 42.0×10⁻¹² M, about 43.0×10⁻¹² M, about 44.0×10⁻¹² M, about 45.0×10⁻¹² M, about 46.0×10⁻¹² M, about 47.0×10⁻¹² M, about 48.0×10⁻¹² M, about 49.0×10⁻¹² M, or about 50.0×10⁻¹² M. In some embodiments, the TSLP-TRAP binds TSLP with a KD between 0.2×10⁻¹² M and 30×10⁻¹² M.

### Method of Making Polypeptides

Also provided herein are methods of producing an polypeptide that include: (a) culturing a cell (e.g., any of the cells described herein) including any of the nucleic acids encoding any of the polypeptides described herein, or any of the expression vectors described herein that include nucleic acid encoding any of the polypeptides described herein, in a culture medium under conditions sufficient to allow for the production of the polypeptide; and (b) harvesting the polypeptide from the host cell or the culture medium. In some embodiments of any of the methods described herein, the method further includes isolating the polypeptide (e.g., through performance of one or more column chromatography steps, ultrafiltration/diafiltration, and/or viral inactivation). In some embodiments of any of the methods described herein, the method further includes formulating the isolated polypeptide into a composition (e.g., a pharmaceutical composition).

Any of the polypeptides described herein can be produced by any cell, (e.g., a mammalian cell). Non-limiting examples of a mammalian cell include: a human cell, a rodent cell (e.g., a rat cell or a mouse cell), a rabbit cell, a dog cell, a cat cell, a porcine cell, or a non-human primate cell. For example, a host cell can be a CHO cell or a HEK cell.

Methods of culturing cells are well known in the art. Cells can be maintained in vitro under conditions that favor cell proliferation, cell growth, and/or cell differentiation. For example, cells can be cultured by contacting a cell (e.g., any of the cells described herein) with a cell culture medium that includes supplemental growth factors to support cell viability and cell growth.

Methods of introducing nucleic acids (e.g., any of the exemplary nucleic acids described herein) and/or expression vectors (e.g., any of the exemplary expression vectors described herein (e.g., an AAV vector)) into cells (e.g., mammalian cells) are known in the art. Non-limiting examples of methods that can be used to introduce a nucleic acid (e.g., DNA, RNA, ssRNA, siRNA, microRNA, or mRNA) and/or an expression vector (e.g., any of the exemplary expression vectors described herein (e.g., an AAV vector) include: electroporation, lipofection, transfection, microinjection, calcium phosphate transfection, dendrimer-based transfection, anionic polymer transfection, cationic polymer transfection, transfection using highly branched organic compounds, cell-squeezing, sonoporation, optical transfection, magnetofection, particle-based transfection (e.g., nanoparticle transfection), transfection using liposomes (e.g., cationic liposomes), and viral transduction (e.g., lentiviral transduction, adenoviral transduction).

Some methods described herein further include isolating or purifying the polypeptide from cell culture medium or from a cell (e.g., a mammalian cell) using techniques well-known in the art (e.g., ion exchange chromatography (anionic or cation), metal-affinity chromatography, ligand-affinity chromatography, size exclusion chromatography, hydrophobic interaction chromatography, and precipitation (e.g., ammonium sulfate precipitation, polyethylene glycol precipitation).

### Method of Treatment

Described herein is the pharmaceutical composition for use in a therapy. In some embodiments, the pharmaceutical compositions may further comprise at least one additional therapeutic agent.

In some embodiments, compositions comprising antibodies can be provided in formulations with a wide variety of pharmaceutically acceptable carriers (see, e.g., Gennaro, Remington: The Science and Practice of Pharmacy with Facts and Comparisons: Drugfacts Plus, 20th ed. (2003): Ansel et al., Pharmaceutical Dosage Forms and Drug Delivery Systems, 7th ed., Lippencott Williams and Wilkins (2004): Kibbe et al., Handbook of Pharmaceutical Excipients, 3rd ed., Pharmaceutical Press (2000)). Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Non-limiting exemplary carriers include saline, buffered saline, dextrose, water, glycerol, ethanol, and combinations thereof.

In some embodiments, compositions comprising the TSLP trap complex may be formulated for injection, including subcutaneous administration, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives. In various embodiments, the compositions may be formulated for inhalation, for example, using pressurized acceptable propellants such as dichlorodifluoromethane, propane, nitrogen, and the like. The compositions may also be formulated, in various embodiments, into sustained release microcapsules, such as with biodegradable or non-biodegradable polymers. A non-limiting exemplary biodegradable formulation includes poly lactic acid-glycolic acid polymer. A non-limiting exemplary non-biodegradable formulation includes a polyglycerin fatty acid ester. Certain methods of making such formulations are described, for example, in EP 1 125 584 A1. In some embodiments, the composition described herein may be manufactured as a medicament for treatment of allergic disease.

### Pharmaceutical Composition

Also provided herein are compositions (e.g., pharmaceutical compositions) that include any of the polypeptides, vectors, or nucleic acids described herein. Any of the pharmaceutical compositions can include any of the polypeptides, vectors, or nucleic acids described herein and one or more (e.g., 1, 2, 3, 4, or 5) pharmaceutically or physiologically acceptable carriers, diluents, or excipients. In some embodiments, any of the pharmaceutical compositions described herein can include one or more buffers (e.g., a neutral-buffered saline, a phosphate-buffered saline (PBS)), one or more carbohydrates (e.g., glucose, mannose, sucrose, dextran, or mannitol), one or more proteins, polypeptides, or amino acids (e.g., glycine), one or more antioxidants, one or more chelating agents (e.g., glutathione or EDTA), one or more preservatives, and/or a pharmaceutically acceptable carrier (e.g., PBS, saline, or bacteriostatic water).

In some embodiments, any of the pharmaceutical compositions described herein can further include one or more (e.g., 1, 2, 3, 4, or 5) agents that promote the entry of any of the vectors or nucleic acids described herein into a cell (e.g., a mammalian cell) (e.g., a liposome or cationic lipid).

In some embodiments, any of the vectors or nucleic acids described herein can be formulated using natural and/or synthetic polymers. Non-limiting examples of polymers that can be included in any of the pharmaceutical compositions described herein can include, but are not limited to: poloxamer, chitosan, dendrimers and poly(lactic-co-glycolic acid) (PLGA) polymers.

The pharmaceutical compositions provided herein can be, e.g., formulated to be compatible with their intended route of administration. In some embodiments, the compositions are formulated for subcutaneous, intramuscular, or intravenous administration. In some examples, the compositions include a therapeutically effective amount of any of the polypeptides, vectors, or nucleic acids described herein. Single or multiple administrations of any of the pharmaceutical compositions described herein can be given (e.g., administered) to a subject depending on, for example, the frequency and the dosage required and tolerated by the subject. A dosage of the pharmaceutical composition including any of the polypeptides described herein, any of the vectors described herein, or any of the nucleic acids described herein should provide a sufficient quantity to effectively ameliorate or treat symptoms, conditions or diseases.

### Kits

A kit-of-parts comprising a pharmaceutical composition together with instructions for use is further provided. For convenience, the kit-of-parts may comprise reagents in predetermined amounts with instructions for use.

In some embodiments, disclosed herein are kits comprising an TSLP-TRAP complex disclosed herein. In some embodiments, a kit can be a diagnostic kit. In some embodiments, a kit comprises an TSLP-TRAP complex disclosed herein and instructions for use. In some embodiments, a kit comprises means for measuring an TSLP-TRAP complex level in a sample and instructions for use. A kit may provide a unit or device for obtaining a sample from a subject (e.g., a device with a needle coupled to an aspirator).

A kit may include a plurality of syringes, ampules, foil packets, or blister packs, each containing a single unit dose of a kit component described herein. Containers of a kit may be airtight, waterproof (e.g., impermeable to changes in moistures or evaporation), and/or light-tight. A kit may include a device suitable for administration of the components, e.g., a syringe, inhalant, pipette, forceps, measured spoon, dropper (e.g., eve dropper), swab (e.g., a cotton swab or wooden swab), or any such delivery device. In some embodiments, the device may be a medical implant device, e.g., packaged for surgical insertion. A kit disclosed herein may comprise one or more reagents or instruments which enable the method to be carried out. In some embodiments, reagents or instruments include one or more of the following: suitable buffer(s) (aqueous solutions), a support comprising wells on which quantitative reactions can be done. A kit may be a specific kit for a specific tissue sample. Further, a kit disclosed herein may comprise a control. In some embodiments, the kit may comprise any of the compositions (e.g., pharmaceutical compositions) described herein which include any of the nucleic acids, any of the polypeptides, or any of the vectors described herein. In some embodiments, a kit can include a solid composition (e.g., a lyophilized composition including any of the vectors, polypeptides, or nucleic acids described herein) and a liquid for solubilizing the lyophilized composition. In some embodiments, the kit includes a vial including any of the pharmaceutical compositions described herein (e.g., formulated as an aqueous pharmaceutical composition).

In addition to the above components, instructions for use may be provided in a kit. These instructions may be presented in the kit in a variety of forms, such as printed information on a suitable medium or substate (e.g., a piece or pieces of paper on which the information is printed), in the packaging of the kit, in a package insert, etc. In some embodiments, instructions for use can be provided on a computer readable medium (e.g., jump/thumb drive, CD, etc.), or which the information has been recorded, or at a website address which may be used via the internet to access the information at a website.

### Device

Another aspect of the disclosure provides a pre-filled syringe or autoinjector device, comprising an TSLP-TRAP complex or a composition described herein. In some embodiments, a composition stored in a container, pre-filled syringe, injector or autoinjector device contains an anti-CD3 antibody or antigen-binding fragment thereof disclosed herein.

### Arrays

Disclosed herein are supports comprising an TSLP-TRAP complex disclosed herein. A support can be a solid support. A support may take a variety of configurations ranging from simple to complex, depending on the intended use of the support. A support can have an overall slide or plate configuration, such as a rectangular or disc configuration. A standard microplate configuration can be used. In some embodiments, the surface may be smooth or substantially planar, or have irregularities, such as depressions or elevations. In some embodiments, a support may have a rectangular cross-sectional shape, having a length of from about 10-200 mm, 40-150 mm, or 75-125 mm; a width of from about 10-200 mm, 20-120 mm, or 25-80 mm, and a thickness of from about 0.01-5.0 mm, 0.1-2 mm, or 0.2 to 1 mm.

In some embodiments, a support can be organic or inorganic; may be metal (e.g., copper or silver) or non-metal; may be a polymer or nonpolymer; may be conducting, semiconducting or nonconducting (insulating); may be reflecting or nonreflecting; may be porous or nonporous; etc. A support as described herein can be formed of any suitable material, including metals, metal oxides, semiconductors, polymers (particularly organic polymers in any suitable form including woven, nonwoven, molded, extruded, cast, etc.), silicon, silicon oxide, and composites thereof. A support can be an array. In some embodiments, a support comprises an array. An array can comprise an ordered spatial arrangement of two or more discrete regions. An array can comprise TSLP-TRAP complexes located at known or unknown discrete regions. Row and column arrangements of arrays can be selected due to the relative simplicity in making such arrangements. The spatial arrangement can, however, be essentially any form selected by the user, and optionally, in a pattern. Areas of an array may be any convenient shape, including circular, ellipsoid, oval, annular, or some other analogously curved shape, where the shape may, in certain embodiments, be a result of the particular method employed to produce the array.

In some embodiments, a support can be planar. In some instances, a support can be spherical. In some instances, a support can be a bead. In some instances, a support can be magnetic. In some embodiments, a magnetic support comprises magnetite, maghemite, FePt, SrFe, iron, cobalt, nickel, chromium dioxide, ferrites, or mixtures thereof. In some embodiments, a support can be nonmagnetic. In some embodiments, the nonmagnetic support can comprise a polymer, metal, glass, alloy, mineral, or mixture thereof. In some instances, a nonmagnetic material can be a coating around a magnetic support. In some instances, a magnetic material may be distributed in the continuous phase of a magnetic material. In some embodiments, the support comprises magnetic and nonmagnetic materials. In some instances, a support can comprise a combination of a magnetic material and a nonmagnetic material. In some embodiments, an TSLP-TRAP complex disclosed herein is directly or indirectly associated with a support disclosed herein.

### Computer Control System

The present disclosure provides computer control systems that are programmed to implement methods of the disclosure. In some embodiments, a computer system is programmed or otherwise configured to interface with an apparatus that is configured to detect TSLP and/or binding of an TSLP-TRAP complex disclosed herein to moiety. The computer system can be an electronic device of a user or a computer system that is remotely located with respect to the electronic device. The electronic device can be a mobile electronic device.

In some embodiments, a computer system includes a central processing unit (CPU, also “processor” and “computer processor” herein, which can be a single core or multi core processor, or a plurality of processors for parallel processing.

In some embodiments, a computer system also includes memory or memory location (e.g., random-access memory, read-only memory, flash memory), electronic storage unit (e.g., hard disk), communication interface (e.g., network adapter) for communicating with one or more other systems, and peripheral devices, such as cache, other memory, data storage and/or electronic display adapters. In some embodiments, the memory, storage unit, interface and peripheral devices are in communication with the CPU through a communication bus, such as a motherboard. In some embodiments, the storage unit can be a data storage unit (or data repository) for storing data. In some embodiments, the computer system is operatively coupled to a computer network (“network”) with the aid of the communication interface. In some embodiments, the network can be the Internet, an internet and/or extranet, or an intranet and/or extranet that is in communication with the Internet. In some embodiments, the network is a telecommunication and/or data network. The network can include one or more computer servers, which can enable distributed computing, such as cloud computing. In some embodiments, the network, in some cases with the aid of the computer system can implement a peer-to-peer network, which may enable devices coupled to the computer system to behave as a client or a server. In some embodiments, the CPU executes a sequence of machine-readable instructions, which can be embodied in a program or software. The instructions may be stored in a memory location, such as the memory. The instructions can be directed to the CPU, which can subsequently program or otherwise configure the CPU to implement methods of the present disclosure. Examples of operations performed by the CPU can include fetch, decode, execute, and writeback. In some embodiments, the CPU can be part of a circuit, such as an integrated circuit. One or more other components of the system can be included in the circuit. In some cases, the circuit is an application specific integrated circuit (ASIC). In some embodiments, the storage unit can store files, such as drivers, libraries and saved programs. The storage unit can store user data, e.g., user preferences and user programs. The computer system in some cases can include one or more additional data storage units that are external to the computer system, such as located on a remote server that is in communication with the computer system through an intranet or the Internet. In some embodiments, the computer system communicates with one or more remote computer systems through the network. For instance, the computer system can communicate with a remote computer system of a user. Examples of remote computer systems include personal computers (e.g., portable PC), slate or tablet PC's telephones, Smart phones, or personal digital assistants. The user can access the computer system via the network.

Methods as described herein can be implemented by way of machine (e.g., computer processor) executable code stored on an electronic storage location of the computer system, such as, for example, on the memory or electronic storage unit. The machine executable or machine-readable code can be provided in the form of software. During use, the code can be executed by the processor. In some cases, the code can be retrieved from the storage unit and stored on the memory for ready access by the processor. In some situations, the electronic storage unit can be precluded, and machine-executable instructions are stored on memory. In some embodiments, the code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

A machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution. In some embodiments, a computer system disclosed herein can include or be in communication with an electronic display that comprises a user interface (UI) for providing, for example, one or more results (immediate results or archived results from a previous experiment), one or more user inputs, reference values from a library or database, or a combination thereof. Examples of Uls include, without limitation, a graphical user interface (GUI) and web-based user interface.

Further, methods and systems of the present disclosure can be implemented by way of one or more algorithms. An algorithm can be implemented by way of software upon execution by the central processing unit. The algorithm can, for example, determine optimized conditions via supervised learning to optimize conditions such as a buffer type, a buffer concentration, a temperature, an incubation period, thresholds, diagnostic/prognostic indications for methods disclosed herein.

### EXAMPLES

The following examples are included for illustrative purposes only and are not intended to limit the scope of the disclosure.

### Example 1: TSLP-TRAP Complex Design and Synthesis

### Design of TSLP-TRAP Complexes

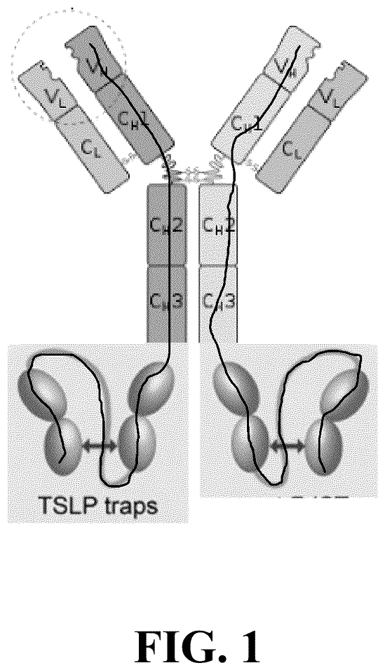

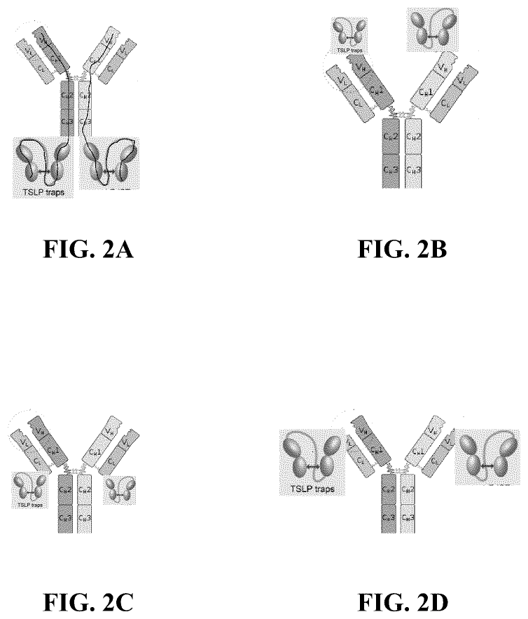

The antigen binding complexes were designed to bind to TSLP, namely the monospecific TSLP-TRAP complex (FIG. 1) or bind TSLP and IL-4R such as the bispecific anti-IL-4R TSLP-TRAP complexes (FIGS. 2-4). The monospecific TSLP-TRAP complexes comprise a TSLP-TRAP associated to a moiety via a linker. The TSLP-TRAP heterodimer comprised the wild-type extracellular domains of TSLPR and IL-7Rα as described in Verstraete, K. et al., Structure and antagonism of the receptor complex mediated by human TSLP in allergy and asthma. Nat. Commun. 8, 14937 doi: 10.1038/ncomms14937 (2017). A glycine serine linker, (GGS)x20, was used to join the TSLPR and IL-7Rα domains to construct the TSLP-TRAP heterodimer described in Verstraete, K. et al. The TSLP-TRAP is arranged as amino acids 25-231 of human TSLPR associated via (GGS)x20 (SEQ ID NO: 3) to amino acids 21-239 of human IL-7Rα (TRAP 1) or amino acids 21-239 of human IL-7Rα associated via (GGS)x20 (SEQ ID NO: 3) to amino acids 25-231 of human TSLPR (TRAP 2). Another glycine serine linker, namely (GGGGS)x4 (SEQ ID NO: 6), was used to join the TSLP-TRAP to the carboxy (C) or amino (N) terminus of a moiety making the antigen binding complex. The moiety component of the TSLP-TRAP (FIG. 1) is linked to is palivizumab, a human IgG4 isotype with a S228P stabilized hinge.

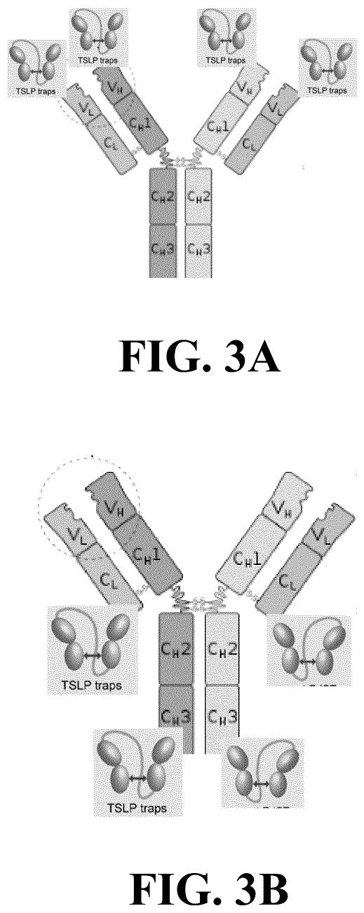

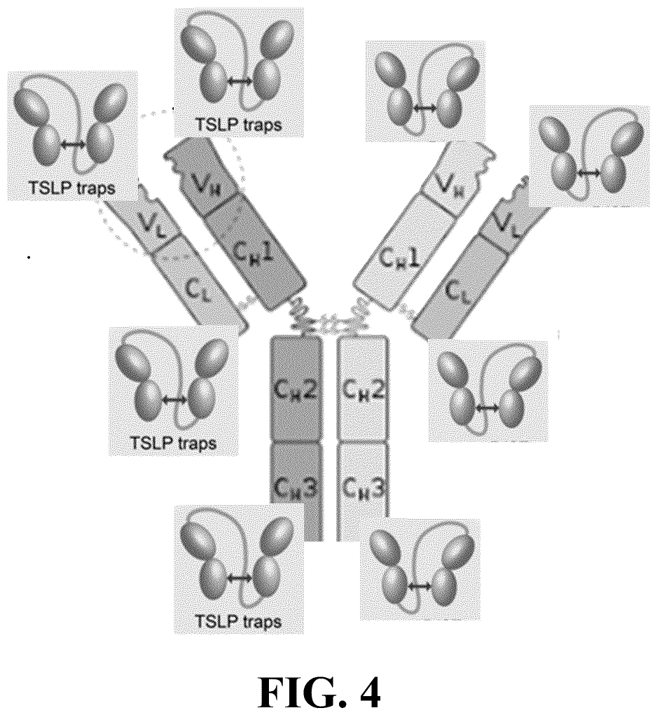

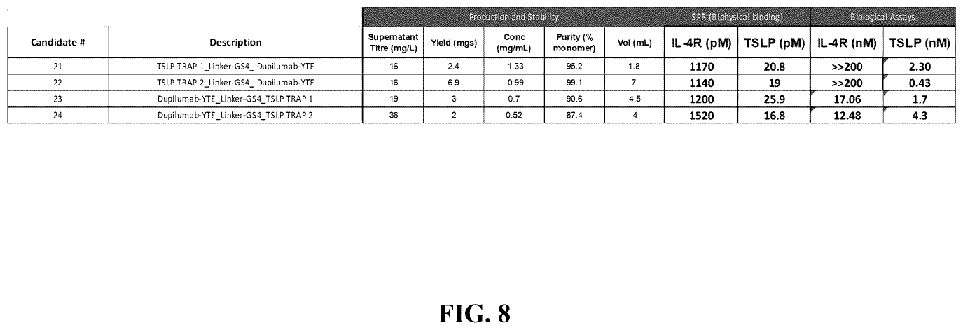

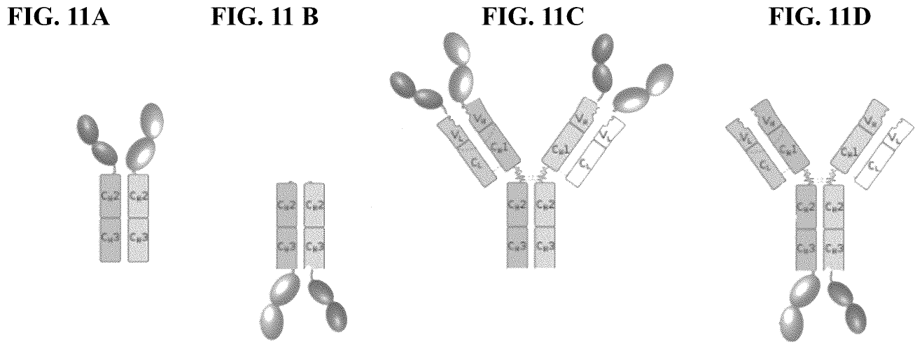

Similarly, the bispecific anti-IL-4R TSLP-TRAP complexes (FIG. 2-FIG. 4) comprise a TSLP-TRAP associated to a moiety via a linker. The moiety component of the bispecific anti-IL-4R TSLP-TRAPs (FIG. 2-FIG. 4) is dupilumab, a human IgG4 isotype that specifically binds to hIL-4R. The complexes have 2 TSLP-TRAPs (FIGS. 2A-2D), 4 TSLP-TRAPS (FIGS. 3A-3B) or 8 TSLP-TRAPS (FIG. 4). The TSLP-TRAP complex can also be formed by linking TSLPR and IL-7Rα independently to an antibody or fragments thereof such that the TSLP-TRAP is formed when folded (FIGS. 11A-11D). The TSLP-TRAP can be associated with N termini (FIG. 11A), C termini (FIG. 11B), N termini (FIG. 11C), or C termini (FIG. 11D) of moieties, for example an antibody or the Fc portion thereof. In some embodiments, a moiety disclosed herein is symmetric and comprises a dimeric assembly of two identical heavy chains. In some embodiments, a moiety disclosed herein is asymmetric. In some embodiments, an asymmetric moiety is generated using knobs-into-holes. In some embodiments, an asymmetric moiety can comprise a construct where amino acids at the contact site between the CH3 domains are substituted by larger or smaller residues forcing a heterodimeric assembly of heavy chains. In some embodiments, the knobs-into-holes approach comprises a knob that is obtained by replacement of a small amino acid with a larger one in the CH3 domain. In some embodiments, the knob is designed to insert into a hole in the CH3 domain that is obtained by replacement of a large residue with a smaller one. In some embodiments, an IL-7Rα and TSLPR may associate when a modification enables two different heavy chains to pair, when IL-7Rα and TSLPR are not directly connected by a linker.

Additional attributes of the complexes include increasing drug serum half-life by introducing M252Y/S254T/T256E (YTE) Fc mutations (EU numbering).

### Synthesis of TSLP-TRAP Complexes

Antigen binding complexes were designed and cloned in appropriate expression vectors. Subsequently, the vectors were transfected in CHO cells. Supernatants from those cells were collected following a two-step purification process involving: 1) purifying the antibodies on MabSelect SuRe Protein A affinity medium and 2) further purifying by size exclusion SEC Purification of HiLoad Superdex 200 pg with a final buffer being PBS with 100 mmol/l L-arginine (pH 6.5-6.7) and subsequent sterile filtration. The quantification for the antigen binding complex's yield was done by absorbance at a280 nm. The monomericity was calculated by HPLC-sec measurement.

### Example 2: Validation of TSLP-TRAP Complex Binding to TSLP and IL-4R

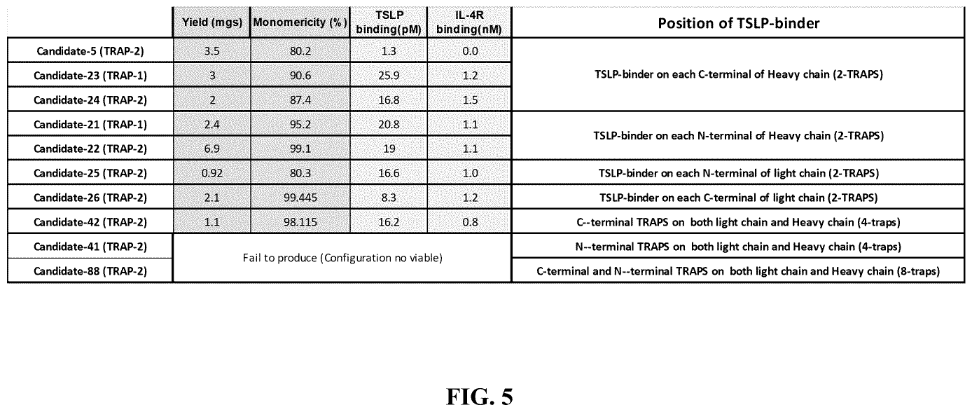

SPR studies were carried out on a Biacore 3000 instrument with a CM5 chip at 25° C. The running buffer comprised 25 mM Hepes, pH 7.5, 150 mM NaCl, 0.5 mg/ml BSA, 0.005% P20. Antibodies were captured via protein A using approximately a 1/50 dilution of each antibody in the running buffer and a 2-minute contact time at a 10 μl/min flow rate. Protein A was immobilized on a CM5 chip via standard amine coupling chemistry in 10 mM acetate pH 4.5. Approximately 800 RU of each antibody was captured. The surfaces were regenerated via a 10 mM glycine pH 1.5 between cycles. Between assays the chip was regenerated with a 2-minute pulse of 100 mM Phosphoric acid. Prior to setting up multicycle kinetic analysis analyte concentration was determined spectrophotometrically using an extinction coefficient of 72,880 M⁻¹ cm⁻¹ for IL4R and 18450 M⁻¹ cm⁻¹ for the TSLP proteins respectively. Analyte samples were prepared by diluting stocks into running buffer to a final concentration of 500 nM for the IL-4R and 25 nM for TSLP followed by serially diluting with a 2-fold series with an association time of 2.5 minutes at a 50 μl/min flow rate. Dissociation data was collected for a period of 1600 seconds. Raw data was analyzed using the Scrubber2 (Biologic Software, AU) program using a double referencing procedure where compound signal is corrected to a blank surface and a buffer injection over the captured antibody surface. Kinetic data was analyzed by fitting to a simple 1:1 binding model. FIG. 5 shows a table summarizing the antigen binding complexes from FIG. 1-FIG. 4 yield (mg), percent monomericity by the method described in Example 1, and their K_{D} to TSLP and IL-4R by the method described above. The candidates are further described in Table 3.

TABLE 3
List of candidates

| | | Full Length |
|---|---|---|
| Candidate | Description | SEQ ID NO: |
| Candidate-5 | Palivizumab-YTE_Linker- | 28 |
| | GS4_TSLP TRAP 2 | |
| Candidate-23 | Dupilumab-YTE_Linker- | 33 |
| | GS4_TSLP TRAP 1 | |
| Candidate-24 | Dupilumab-YTE_Linker- | 34 |
| | GS4_TSLP TRAP 2 | |
| Candidate-21 | TSLP TRAP 1_Linker- | 31 |
| | GS4_Dupilumab-YTE | |
| Candidate-22 | TSLP TRAP 2_Linker- | 32 |
| | GS4_Dupilumab-YTE | |
| Candidate-25 | Light chain N-terminal | 35 |
| | TRAP: TSLP TRAP | |
| | 2_Linker-GS4_Dupilumab- | |
| | YTE [2 × TRAP 2] | |
| Candidate-26 | Light chain C-terminal | 36 |
| | TRAP: Dupilumab- | |
| | YTE_Linker-GS4_TSLP | |
| | TRAP 2 [2 × TRAP 2] | |
| Candidate-42 | Heavy and Light chain C- | 38 |
| | terminal TRAP: Dupilumab- | |
| | YTE_Linker-GS4_TSLP | |
| | TRAP 2 [4 × TRAP 2] | |
| Candidate-41 | Heavy and Light chain N- | 37 |
| | terminal TRAP: TSLP TRAP | |
| | 2_Linker-GS4_Dupilumab- | |
| | YTE [4 × TRAP 2] | |
| Candidate-88 | Heavy and Light chain N- | 39 |
| | terminal and C-terminal | |
| | TRAP: TSLP TRAP | |
| | 2_Linker-GS4_Dupilumab- | |
| | YTE_Linker-GS4_TSLP | |
| | TRAP 2 [8 × TRAP 2] | |
| A variant of | A Variant of Light chain C- | 45 |
| Candidate 26 | terminal TRAP: Dupilumab- | |
| | YTE_Linker-GS4_TSLP | |
| | TRAP 2 [2 × TRAP 2] | |
| A variant of | A Variant of Heavy and Light | 46 |
| Candidate 42 | chain C-terminal TRAP: | |
| | Dupilumab-YTE_Linker- | |
| | GS4_TSLP TRAP 2 [4 × | |
| | TRAP 2] | |
| A variant of | A Variant of Heavy and Light | 47 |
| Candidate 42 | chain C-terminal TRAP: | |
| | Dupilumab-YTE_Linker- | |
| | GS4_TSLP TRAP 2 [4 × | |
| | TRAP 2] | |
| Candidate 0001 | Dupilumab-YTE | 48 |
| A variant of | A variant of TSLP TRAP | 50 |
| Candidate 21 | 1_Linker-GS4_Dupilumab- | |
| | YTE | |

### Example 3: In Vitro Validation of TSLP-TRAP Complex Binding to TSLP and IL-4R

To further determine positioning of the TSLP-Trap to achieve a viable and fully bifunctional molecule, biological assays were used to characterize the in vitro potency of each molecule. Molecules were tested in human donor PBMC (peripheral blood mononuclear cells) for measuring their ability to block Il-4 mediated responses and TSLP-mediated responses.

IL-4 mediated responses were assessed by measuring CD23 upregulation in B-cells in response to Il-4 treatment. CD23 upregulation was measured by FACS. Frozen PBMCs were thawed and incubated for 60 minutes in a humidified incubator at 37° C. and 5% CO₂. 100 microliters of cell suspension at 1×10⁷ cells/mL were added to a 96-well flat bottom plate. Each well contained about 1×10⁶ cells. 50 μL/well of 4× stock of IL-4 was added to make a final concentration of 3 ng/mL. Cells were incubated for 40 hours at 37° C. in a humidified CO₂ incubator set to 5% CO₂. Antibody cocktails of readout antibodies for FACS (CD23, CD19) and isotype controls for the number of wells that needed to be stained in FACS buffer were prepared according to manufacturer instructions. Cells were transferred to 96-well round bottom plates and washed twice with PBS. Cells were resuspended in PBS and stained with 100 μL of Zombie LIVE/DEAD stain according to the manufacturer's instructions. The cells were incubated for 20 minutes at room temperature and protected from light. The cells were spun down to remove the Zombie AQUA LIVE/DEAD stain. The cells were washed once with PBS. The cells were resuspended in 50 μL Fc blocking reagent for 10 minutes at room temperature. 50 μL of the antibody cocktail was added to the wells and the sample were incubated for 25 minutes at 4° C. protected from light. The cells were washed with PBS to remove unbound antibodies. The samples were acquired by a flow cytometer and at least 10000 events of the CD23+ population was acquired per sample.

TSLP mediated responses were assessed by measuring the secretion of Thymus and activation regulated chemokine (TARC) in human PBMCs by Meso Scale Discovery (MSD). Frozen PBMCs were thawed and incubated for 60 minutes in a humidified incubator at 37° C. and 5% CO₂. 100 μLs of cell suspension at 1×10⁷ cells/mL were added to a 96-well flat bottom plate. Each well contained about 1×10⁶ cells. 100 μL/well of the 2× stock of serially diluted recombinant human TSLP was added. Cells were incubated for 24 hours at 37° C. in a humidified CO₂ incubator set to 5% CO₂. Plates were centrifuged at 1300 rpm for 5 minutes and at least 120) μL of supernatants were collected for TARC. The plates were washed with 150 μL/well of Tris Wash Buffer. 50 μL of samples were added per well. The plates were sealed with an adhesive plate seal and incubated for 2 hours with shaking (300-1000 rpm) at room temperature. The plates were washed 3 times with 150 μL/well of Tris Wash Buffer. 25 μL of detection antibody solution was added to each well. The plate was sealed with an adhesive plate seal and incubated for 2 hours with shaking 300-1000 rpm) at room temperature. The plates were washed 3 times with 150 μL/well of Tris Wash Buffer. 150 μL of 2× read Buffer T to each well of the MSD plate and incubated at room temperature for 10 minutes.

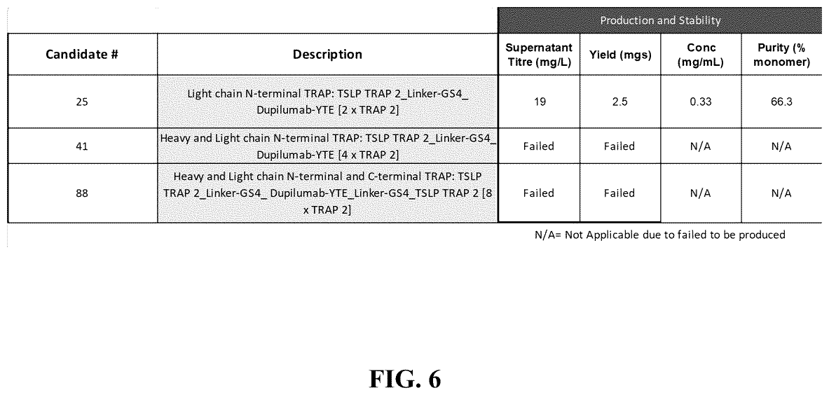

When the TSLP-TRAP was positioned at the N-terminal of the light chain, the resulting molecule could not be produced (FIG. 6—example candidate 41 and candidate-88) or when some production was achieved it showed that the resulting molecule was unstable in solution as measured by the percent of monomeric non-aggregated molecules in the solution (FIG. 6—example candidate 25 which showed only 66.3% monomericity).

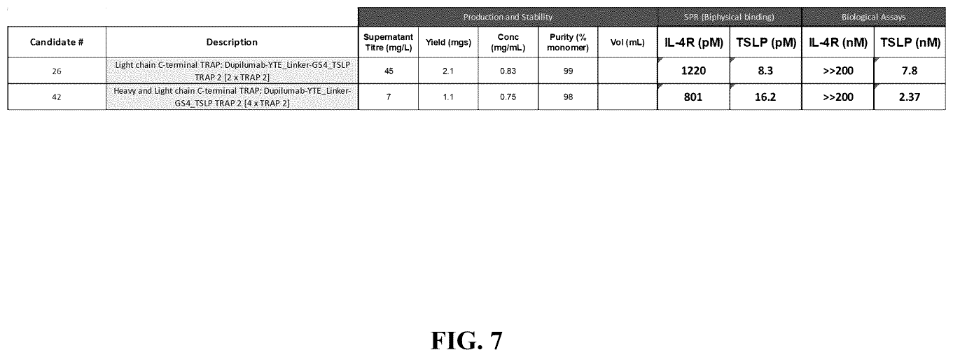

When TSLP-TRAP is positioned at the C-terminal of the light chain, the molecule could be produced at adequate concentration and stability as measured by percent monomericity (FIG. 7—example: candidate-26 showed 99% monomericity and candidate-42 showed 98% monomericity). The molecules were able to block TSL responses in human PBMCs as part of the biological assay testing (FIG. 7—for example candidate-26 inhibited TSLP responses with an IC₅₀=7.8 nM and candidate-42 with an IC₅₀=2.37 nM). However, the molecule did not block IL-4 responses in the PBMC biological assays. The same molecule showed binding to soluble IL-4Rα in biophysical assays.

When TSLP trap was positioned at the N-terminal end of the heavy chain, for instance, candidate-21 and candidate-22 were able to express well, have excellent stability and bind to both IL4R and TSLP using biophysical assays (FIG. 8). Both candidates also showed excellent potency on PBMC assays in response to TSLP. Both candidates ablated TSLP with low nM potency (IC₅₀=2.3 nM for candidate-21) or sub-nanomolar potency (IC₅₀=0.43 nM for candidate-22). The potency of both candidates was similar to that recorded for the anti-TSLP antibody Tezepelumab on the same assay (IC₅₀=1.6 nM). However, neither of candidates showed activity in the IL-4 biological assays (IC₅₀>>>200 nM) while the anti-IL-4R antibody dupilumab demonstrated robust inhibition when tested as a benchmark (IC₅₀=4.7 nM).

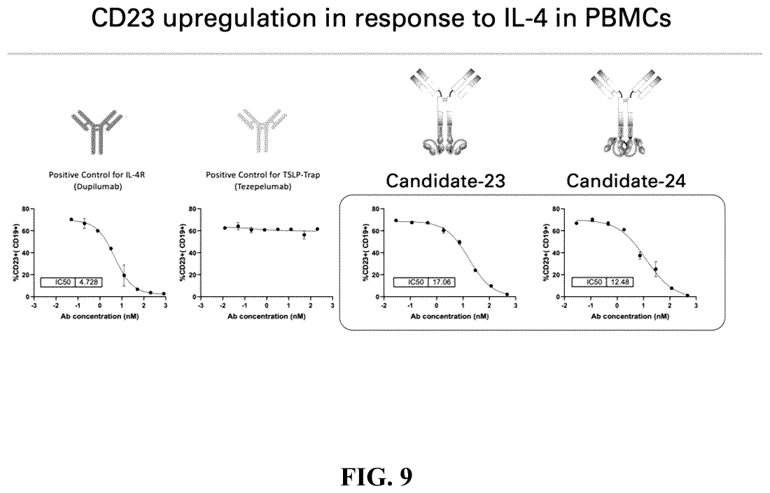

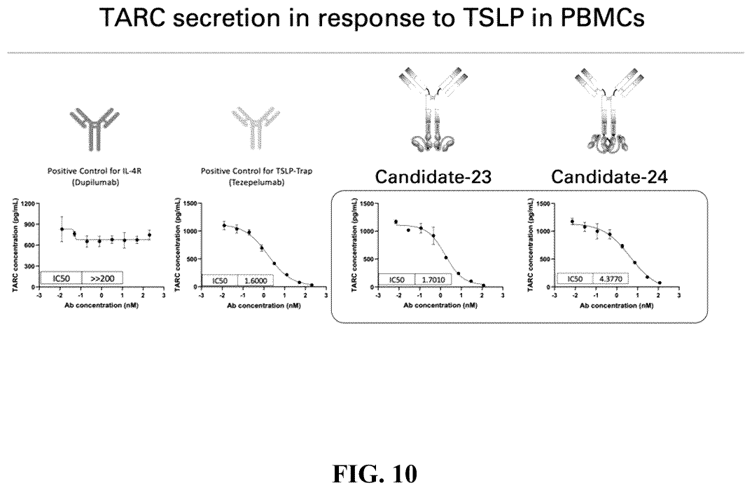

When TSLP-TRAP was positioned at the C-terminal of the heavy chain, the molecule could be produced at adequate concentrations and with excellent stability as measured by high monomericity (FIG. 8—for instance 90.6% for candidate-23 and 87% for candidate-24). The molecules also inhibited both the upregulation of CD23 in response to IL-4 in IL-4 biological assays with nM potency (FIG. 9—for example an IC₅₀=17.06 nM for candidate-23 and 12.48 nM for candidate-24) and TARC secretion in response to TSLP with nM potency (FIG. 10—for example an IC₅₀=1.7 nM for candidate-23 and 4.3 nM for candidate-24).

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

Sequences

| Construct | Description | SEQ ID NO: | Sequence |
|---|---|---|---|
| Palivizumab- | Full Length | 27 | QVTLRESGPALVKPTQTLTLTCTFSGFSLST |
| YTE Linker- | Sequence | | SGMSVGWIRQPPGKALEWLADIWWDDKK |
| GS4_TSLP | | | DYNPSLKSRLTISKDTSANQVVLKVTNMDP |
| TRAP 1 | | | ADTATYYCARSMITNWYFDVWGAGTTVT |
| | | | VSSASTKGPSVFPLAPCSRSTSESTAALGCL |
| | | | VKDYFPEPVTVSWNSGALTSGVHTFPAVL |
| | | | QSSGLYSLSSVVTVPSSSLGTKTYTCNVDH |
| | | | KPSNTKVDKRVESKYGPPCPPCPAPEFLGG |
| | | | PSVFLFPPKPKDTLYITREPEVTCVVVDVSQ |
| | | | EDPEVQFNWYVDGVEVHNAKTKPREEQF |
| | | | NSTYRVVSVLTVLHQDWLNGKEYKCKVS |
| | | | NKGLPSSIEKTISKAKGQPREPQVYTLPPSQ |
| | | | EEMTKNQVSLTCLVKGFYPSDIAVEWESN |
| | | | GQPENNYKTTPPVLDSDGSFFLYSRLTVDK |
| | | | SRWQEGNVFSCSVMHEALHNHYTQKSLSL |
| | | | SLGGGGGSGGGGSGGGGSGGGGSGAAEG |
| | | | VQIQIIYFNLETVQVTWNASKYSRTNLTFH |
| | | | YRFNGDEAYDQCTNYLLQEGHTSGCLLDA |
| | | | EQRDDILYFSIRNGTHPVFTASRWMVYYLK |
| | | | PSSPKHVRFSWHQDAVTVTCSDLSYGDLL |
| | | | YEVQYRSPFDTEWQSKQENTCNVTIEGLD |
| | | | AEKCYSFWVRVKAMEDVYGPDTYPSDWS |
| | | | EVTCWQRGEIRDACAETPTPPKPKLSKGGS |
| | | | GGSGGSGGSGGSGGSGGSGGSGGSGGSGG |
| | | | SGGSGGSGGSGGSGGSGGSGGSGGSGGSE |
| | | | SGYAQNGDLEDAELDDYSFSCYSQLEVNG |
| | | | SQHSLTCAFEDPDVNITNLEFEICGALVEVK |
| | | | CLNFRKLQEIYFIETKKFLLIGKSNICVKVG |
| | | | EKSLTCKKIDLTTIVKPEAPFDLSVVYREGA |
| | | | NDFVVTFNTSHLQKKYVKVLMHDVAYRQ |
| | | | EKDENKWTHVNLSSTKLTLLQRKLQPAAM |
| | | | YEIKVRSIPDHYFKGFWSEWSPSYYFRTPEI |
| | | | NNSSGEMDDIQMTQSPSTLSASVGDRVTIT |
| | | | CKCQLSVGYMHWYQQKPGKAPKLLIYDTS |
| | | | KLASGVPSRFSGSGSGTAFTLTISSLQPDDF |
| | | | ATYYCFQGSGYPFTFGGGTKLEIKRTVAAP |
| | | | SVFIFPPSDEQLKSGTASVVCLLNNFYPREA |
| | | | KVQWKVDNALQSGNSQESVTEQDSKDST |
| | | | YSLSSTLTLSKADYEKHKVYACEVTHQGL |
| | | | SSPVTKSFNRGEC |
| | VH Domain | 20 | QVTLRESGPALVKPTQTLTLTCTFSGFSLST |
| | | | SGMSVGWIRQPPGKALEWLADIWWDDKK |
| | | | DYNPSLKSRLTISKDTSANQVVLKVINMDP |
| | | | ADTATYYCARSMITNWYFDVWGAGTTVT |
| | | | VSS |
| | VL Domain | 25 | DIQMTQSPSTLSASVGDRVTITCKCQLSVG |
| | | | YMHWYQQKPGKAPKLLIYDTSKLASGVPS |
| | | | RFSGSGSGTAFTLTISSLQPDDFATYYCFQG |
| | | | SGYPFTFGGGTKLEIK |
| | First Linker | 6 | GGGGSGGGGSGGGGSGGGGS |
| | Second Linker | 3 | GGSGGSGGSGGSGGSGGSGGSGGSGGSGG |
| | | | SGGSGGSGGSGGSGGSGGSGGSGGSGGSG |
| | | | GS |
| | HCDR1 | 17 | GFSLSTSGMS |
| | HCDR2 | 18 | IWWDDKK |
| | HCDR3 | 19 | ARSMITNWYFDV |
| | LCDR1 | 22 | LSVGY |
| | LCDR2 | 23 | DTS |
| | LCDR3 | 24 | FQGSGYPFT |
| | TSLP-TRAP | 4 | GAAEGVQIQIIYFNLETVQVTWNASKYSRT |
| | | | NLTFHYRFNGDEAYDQCTNYLLQEGHTSG |
| | | | CLLDAEQRDDILYFSIRNGTHPVFTASRWM |
| | | | VYYLKPSSPKHVRFSWHQDAVTVTCSDLS |
| | | | YGDLLYEVQYRSPFDTEWQSKQENTCNVT |
| | | | IEGLDAEKCYSFWVRVKAMEDVYGPDTYP |
| | | | SDWSEVTCWQRGEIRDACAETPTPPKPKLS |
| | | | KGGSGGSGGSGGSGGSGGSGGSGGSGGSG |
| | | | GSGGSGGSGGSGGSGGSGGSGGSGGSGGS |
| | | | GGSESGYAQNGDLEDAELDDYSFSCYSQL |
| | | | EVNGSQHSLTCAFEDPDVNITNLEFEICGAL |
| | | | VEVKCLNFRKLQEIYFIETKKFLLIGKSNIC |
| | | | VKVGEKSLTCKKIDLTTIVKPEAPFDLSVV |
| | | | YREGANDFVVTFNTSHLQKKYVKVLMHD |
| | | | VAYRQEKDENKWTHVNLSSTKLTLLQRKL |
| | | | QPAAMYEIKVRSIPDHYFKGFWSEWSPSY |
| | | | YFRTPEINNSSGEMD |
| Palivizumab- | Full Length | 28 | QVTLRESGPALVKPTQTLTLTCTFSGFSLST |
| YTE Linker- | Sequence | | SGMSVGWIRQPPGKALEWLADIWWDDKK |
| GS4_TSLP | | | DYNPSLKSRLTISKDTSANQVVLKVTNMDP |
| TRAP 2 | | | ADTATYYCARSMITNWYFDVWGAGTTVT |
| | | | VSSASTKGPSVFPLAPCSRSTSESTAALGCL |
| | | | VKDYFPEPVTVSWNSGALTSGVHTFPAVL |
| | | | QSSGLYSLSSVVTVPSSSLGTKTYTCNVDH |
| | | | KPSNTKVDKRVESKYGPPCPPCPAPEFLGG |
| | | | PSVFLFPPKPKDTLYITREPEVTCVVVDVSQ |
| | | | EDPEVQFNWYVDGVEVHNAKTKPREEQF |
| | | | NSTYRVVSVLTVLHQDWLNGKEYKCKVS |
| | | | NKGLPSSIEKTISKAKGQPREPQVYTLPPSQ |
| | | | EEMTKNQVSLTCLVKGFYPSDIAVEWESN |
| | | | GQPENNYKTTPPVLDSDGSFFLYSRLTVDK |
| | | | SRWQEGNVFSCSVMHEALHNHYTQKSLSL |
| | | | SLGGGGGSGGGGSGGGGSGGGGSESGYAQ |
| | | | NGDLEDAELDDYSFSCYSQLEVNGSQHSLT |
| | | | CAFEDPDVNITNLEFEICGALVEVKCLNFR |
| | | | KLQEIYFIETKKFLLIGKSNICVKVGEKSLT |
| | | | CKKIDLTTIVKPEAPFDLSVVYREGANDFV |
| | | | VTFNTSHLQKKYVKVLMHDVAYRQEKDE |
| | | | NKWTHVNLSSTKLTLLQRKLQPAAMYEIK |
| | | | VRSIPDHYFKGFWSEWSPSYYFRTPEINNSS |
| | | | GEMDGGSGGSGGSGGSGGSGGSGGSGGSG |
| | | | GSGGSGGSGGSGGSGGSGGSGGSGGSGGS |
| | | | GGSGGSGAAEGVQIQIIYFNLETVQVTWNA |
| | | | SKYSRTNLTFHYRFNGDEAYDQCTNYLLQ |
| | | | EGHTSGCLLDAEQRDDILYFSIRNGTHPVFT |
| | | | ASRWMVYYLKPSSPKHVRFSWHQDAVTV |
| | | | TCSDLSYGDLLYEVQYRSPFDTEWQSKQE |
| | | | NTCNVTIEGLDAEKCYSFWVRVKAMEDV |
| | | | YGPDTYPSDWSEVTCWQRGEIRDACAETP |
| | | | TPPKPKLSKDIQMTQSPSTLSASVGDRVTIT |
| | | | CKCQLSVGYMHWYQQKPGKAPKLLIYDTS |
| | | | KLASGVPSRFSGSGSGTAFTLTISSLQPDDF |
| | | | ATYYCFQGSGYPFTFGGGTKLEIKRTVAAP |
| | | | SVFIFPPSDEQLKSGTASVVCLLNNFYPREA |
| | | | KVQWKVDNALQSGNSQESVTEQDSKDST |
| | | | YSLSSTLTLSKADYEKHKVYACEVTHQGL |
| | | | SSPVTKSFNRGEC |
| | VH Domain | 20 | QVTLRESGPALVKPTQTLTLTCTFSGFSLST |
| | | | SGMSVGWIRQPPGKALEWLADIWWDDKK |
| | | | DYNPSLKSRLTISKDTSANQVVLKVINMDP |
| | | | ADTATYYCARSMITNWYFDVWGAGTTVT |
| | | | VSS |
| | VL Domain | 25 | DIQMTQSPSTLSASVGDRVTITCKCQLSVG |
| | | | YMHWYQQKPGKAPKLLIYDTSKLASGVPS |
| | | | RFSGSGSGTAFTLTISSLQPDDFATYYCFQG |
| | | | SGYPFTFGGGTKLEIK |
| | First Linker | 6 | GGGGSGGGGSGGGGSGGGGS |
| | Second linker | 3 | GGSGGSGGSGGSGGSGGSGGSGGSGGSGG |
| | | | SGGSGGSGGSGGSGGSGGSGGSGGSGGSG |
| | | | GS |
| | HCDR1 | 17 | GFSLSTSGMS |
| | HCDR2 | 18 | IWWDDKK |
| | HCDR3 | 19 | ARSMITNWYFDV |
| | LCDR1 | 22 | LSVGY |
| | LCDR2 | 23 | DTS |
| | LCDR3 | 24 | FQGSGYPFT |
| | TSLP-TRAP | 5 | ESGYAQNGDLEDAELDDYSFSCYSQLEVN |
| | | | GSQHSLTCAFEDPDVNITNLEFEICGALVEV |
| | | | KCLNFRKLQEIYFIETKKFLLIGKSNICVKV |
| | | | GEKSLTCKKIDLTTIVKPEAPFDLSVVYREG |
| | | | ANDFVVTFNTSHLQKKYVKVLMHDVAYR |
| | | | QEKDENKWTHVNLSSTKLTLLQRKLQPAA |
| | | | MYEIKVRSIPDHYFKGFWSEWSPSYYFRTP |
| | | | EINNSSGEMDGGSGGSGGSGGSGGSGGSG |
| | | | GSGGSGGSGGSGGSGGSGGSGGSGGSGGS |
| | | | GGSGGSGGSGGSGAAEGVQIQIIYFNLETV |
| | | | QVTWNASKYSRTNLTFHYRFNGDEAYDQ |
| | | | CTNYLLQEGHTSGCLLDAEQRDDILYFSIR |
| | | | NGTHPVFTASRWMVYYLKPSSPKHVRFSW |
| | | | HQDAVTVTCSDLSYGDLLYEVQYRSPFDT |
| | | | EWQSKQENTCNVTIEGLDAEKCYSFWVRV |
| | | | KAMEDVYGPDTYPSDWSEVTCWQRGEIR |
| | | | DACAETPTPPKPKLSK |
| TSLP | Full Length | 29 | GAAEGVQIQIIYFNLETVQVTWNASKYSRT |
| TRAP | Sequence | | NLTFHYRFNGDEAYDQCTNYLLQEGHTSG |
| 1_Linker- | | | CLLDAEQRDDILYFSIRNGTHPVFTASRWM |
| GS4_ | | | VYYLKPSSPKHVRFSWHQDAVTVTCSDLS |
| Palivizumab- | | | YGDLLYEVQYRSPFDTEWQSKQENTCNVT |
| YTE | | | IEGLDAEKCYSFWVRVKAMEDVYGPDTYP |
| | | | SDWSEVTCWQRGEIRDACAETPTPPKPKLS |
| | | | KGGSGGSGGSGGSGGSGGSGGSGGSGGSG |
| | | | GSGGSGGSGGSGGSGGSGGSGGSGGSGGS |
| | | | GGSESGYAQNGDLEDAELDDYSFSCYSQL |
| | | | EVNGSQHSLTCAFEDPDVNITNLEFEICGAL |
| | | | VEVKCLNFRKLQEIYFIETKKFLLIGKSNIC |
| | | | VKVGEKSLTCKKIDLTTIVKPEAPFDLSVV |
| | | | YREGANDFVVTFNTSHLQKKYVKVLMHD |
| | | | VAYRQEKDENKWTHVNLSSTKLTLLQRKL |
| | | | QPAAMYEIKVRSIPDHYFKGFWSEWSPSY |
| | | | YFRTPEINNSSGEMDGGGGSGGGGSGGGG |
| | | | SGGGGSQVTLRESGPALVKPTQTLTLTCTF |
| | | | SGFSLSTSGMSVGWIRQPPGKALEWLADI |
| | | | WWDDKKDYNPSLKSRLTISKDTSANQVVL |
| | | | KVTNMDPADTATYYCARSMITNWYFDVW |
| | | | GAGTTVTVSSASTKGPSVFPLAPCSRSTSES |
| | | | TAALGCLVKDYFPEPVTVSWNSGALTSGV |
| | | | HTFPAVLQSSGLYSLSSVVTVPSSSLGTKTY |
| | | | TCNVDHKPSNTKVDKRVESKYGPPCPPCP |
| | | | APEFLGGPSVFLFPPKPKDTLYITREPEVTC |
| | | | VVVDVSQEDPEVQFNWYVDGVEVHNAKT |
| | | | KPREEQFNSTYRVVSVLTVLHQDWLNGKE |
| | | | YKCKVSNKGLPSSIEKTISKAKGQPREPQV |
| | | | YTLPPSQEEMTKNQVSLTCLVKGFYPSDIA |
| | | | VEWESNGQPENNYKTTPPVLDSDGSFFLYS |
| | | | RLTVDKSRWQEGNVFSCSVMHEALHNHY |
| | | | TQKSLSLSLGDIQMTQSPSTLSASVGDRVTI |
| | | | TCKCQLSVGYMHWYQQKPGKAPKLLIYD |
| | | | TSKLASGVPSRFSGSGSGTAFTLTISSLQPD |
| | | | DFATYYCFQGSGYPFTFGGGTKLEIKRTVA |
| | | | APSVFIFPPSDEQLKSGTASVVCLLNNFYPR |
| | | | EAKVQWKVDNALQSGNSQESVTEQDSKD |
| | | | STYSLSSTLTLSKADYEKHKVYACEVTHQ |
| | | | GLSSPVTKSFNRGEC |
| | VH Domain | 20 | QVTLRESGPALVKPTQTLTLTCTFSGFSLST |
| | | | SGMSVGWIRQPPGKALEWLADIWWDDKK |
| | | | DYNPSLKSRLTISKDTSANQVVLKVTNMDP |
| | | | ADTATYYCARSMITNWYFDVWGAGTTVT |
| | | | VSS |
| | VL Domain | 25 | DIQMTQSPSTLSASVGDRVTITCKCQLSVG |
| | | | YMHWYQQKPGKAPKLLIYDTSKLASGVPS |
| | | | RFSGSGSGTAFTLTISSLQPDDFATYYCFQG |
| | | | SGYPFTFGGGTKLEIK |
| | First Linker | 6 | GGGGSGGGGSGGGGSGGGGS |
| | Second Linker | 3 | GGSGGSGGSGGSGGSGGSGGSGGSGGSGG |
| | | | SGGSGGSGGSGGSGGSGGSGGSGGSGGSG |
| | | | GS |
| | HCDR1 | 17 | GFSLSTSGMS |
| | HCDR2 | 18 | IWWDDKK |
| | HCDR3 | 19 | ARSMITNWYFDV |
| | LCDR1 | 22 | LSVGY |
| | LCDR2 | 23 | DTS |
| | LCDR3 | 24 | FQGSGYPFT |
| | TSLP-TRAP | 4 | GAAEGVQIQIIYFNLETVQVTWNASKYSRT |
| | | | NLTFHYRFNGDEAYDQCTNYLLQEGHTSG |
| | | | CLLDAEQRDDILYFSIRNGTHPVFTASRWM |
| | | | VYYLKPSSPKHVRFSWHQDAVTVTCSDLS |
| | | | YGDLLYEVQYRSPFDTEWQSKQENTCNVT |
| | | | IEGLDAEKCYSFWVRVKAMEDVYGPDTYP |
| | | | SDWSEVTCWQRGEIRDACAETPTPPKPKLS |
| | | | KGGSGGSGGSGGSGGSGGSGGSGGSGGSG |
| | | | GSGGSGGSGGSGGSGGSGGSGGSGGSGGS |
| | | | GGSESGYAQNGDLEDAELDDYSFSCYSQL |
| | | | EVNGSQHSLTCAFEDPDVNITNLEFEICGAL |
| | | | VEVKCLNFRKLQEIYFIETKKFLLIGKSNIC |
| | | | VKVGEKSLTCKKIDLTTIVKPEAPFDLSVV |
| | | | YREGANDFVVTFNTSHLQKKYVKVLMHD |
| | | | VAYRQEKDENKWTHVNLSSTKLTLLQRKL |
| | | | QPAAMYEIKVRSIPDHYFKGFWSEWSPSY |
| | | | YFRTPEINNSSGEMD |
| TSLP | Full Length | 30 | ESGYAQNGDLEDAELDDYSFSCYSQLEVN |
| TRAP | Sequence | | GSQHSLTCAFEDPDVNITNLEFEICGALVEV |
| 2_Linker- | | | KCLNFRKLQEIYFIETKKFLLIGKSNICVKV |
| GS4_ | | | GEKSLTCKKIDLTTIVKPEAPFDLSVVYREG |
| Palivizumab- | | | ANDFVVTFNTSHLQKKYVKVLMHDVAYR |
| YTE | | | QEKDENKWTHVNLSSTKLTLLQRKLQPAA |
| | | | MYEIKVRSIPDHYFKGFWSEWSPSYYFRTP |
| | | | EINNSSGEMDGGSGGSGGSGGSGGSGGSG |
| | | | GSGGSGGSGGSGGSGGSGGSGGSGGSGGS |
| | | | GGSGGSGGSGGSGAAEGVQIQIIYFNLETV |
| | | | QVTWNASKYSRTNLTFHYRFNGDEAYDQ |
| | | | CTNYLLQEGHTSGCLLDAEQRDDILYFSIR |
| | | | NGTHPVFTASRWMVYYLKPSSPKHVRFSW |
| | | | HQDAVTVTCSDLSYGDLLYEVQYRSPFDT |
| | | | EWQSKQENTCNVTIEGLDAEKCYSFWVRV |
| | | | KAMEDVYGPDTYPSDWSEVTCWQRGEIR |
| | | | DACAETPTPPKPKLSKGGGGSGGGGSGGG |
| | | | GSGGGGSQVTLRESGPALVKPTQTLTLTCT |
| | | | FSGFSLSTSGMSVGWIRQPPGKALEWLADI |
| | | | WWDDKKDYNPSLKSRLTISKDTSANQVVL |
| | | | KVTNMDPADTATYYCARSMITNWYFDVW |
| | | | GAGTTVTVSSASTKGPSVFPLAPCSRSTSES |
| | | | TAALGCLVKDYFPEPVTVSWNSGALTSGV |
| | | | HTFPAVLQSSGLYSLSSVVTVPSSSLGTKTY |
| | | | TCNVDHKPSNTKVDKRVESKYGPPCPPCP |
| | | | APEFLGGPSVFLFPPKPKDTLYITREPEVTC |
| | | | VVVDVSQEDPEVQFNWYVDGVEVHNAKT |
| | | | KPREEQFNSTYRVVSVLTVLHQDWLNGKE |
| | | | YKCKVSNKGLPSSIEKTISKAKGQPREPQV |
| | | | YTLPPSQEEMTKNQVSLTCLVKGFYPSDIA |
| | | | VEWESNGQPENNYKTTPPVLDSDGSFFLYS |
| | | | RLTVDKSRWQEGNVFSCSVMHEALHNHY |
| | | | TQKSLSLSLGDIQMTQSPSTLSASVGDRVTI |
| | | | TCKCQLSVGYMHWYQQKPGKAPKLLIYD |
| | | | TSKLASGVPSRFSGSGSGTAFTLTISSLQPD |
| | | | DFATYYCFQGSGYPFTFGGGTKLEIKRTVA |
| | | | APSVFIFPPSDEQLKSGTASVVCLLNNFYPR |
| | | | EAKVQWKVDNALQSGNSQESVTEQDSKD |
| | | | STYSLSSTLTLSKADYEKHKVYACEVTHQ |
| | | | GLSSPVTKSFNRGEC |
| | VH Domain | 20 | QVTLRESGPALVKPTQTLTLTCTFSGFSLST |
| | | | SGMSVGWIRQPPGKALEWLADIWWDDKK |
| | | | DYNPSLKSRLTISKDTSANQVVLKVTNMDP |
| | | | ADTATYYCARSMITNWYFDVWGAGTTVT |
| | | | VSS |
| | VL Domain | 25 | DIQMTQSPSTLSASVGDRVTITCKCQLSVG |
| | | | YMHWYQQKPGKAPKLLIYDTSKLASGVPS |
| | | | RFSGSGSGTAFTLTISSLQPDDFATYYCFQG |
| | | | SGYPFTFGGGTKLEIK |
| | First Linker | 6 | GGGGSGGGGSGGGGSGGGGS |
| | Second Linker | 3 | GGSGGSGGSGGSGGSGGSGGSGGSGGSGG |
| | | | SGGSGGSGGSGGSGGSGGSGGSGGSGGSG |
| | | | GS |
| | HCDR1 | 17 | GFSLSTSGMS |
| | HCDR2 | 18 | IWWDDKK |
| | HCDR3 | 19 | ARSMITNWYFDV |
| | LCDR1 | 22 | LSVGY |
| | LCDR2 | 23 | DTS |
| | LCDR3 | 24 | FQGSGYPFT |
| | TSLP-TRAP | 5 | ESGYAQNGDLEDAELDDYSFSCYSQLEVN |
| | | | GSQHSLTCAFEDPDVNITNLEFEICGALVEV |
| | | | KCLNFRKLQEIYFIETKKFLLIGKSNICVKV |
| | | | GEKSLTCKKIDLTTIVKPEAPFDLSVVYREG |
| | | | ANDFVVTFNTSHLQKKYVKVLMHDVAYR |
| | | | QEKDENKWTHVNLSSTKLTLLQRKLQPAA |
| | | | MYEIKVRSIPDHYFKGFWSEWSPSYYFRTP |
| | | | EINNSSGEMDGGSGGSGGSGGSGGSGGSG |
| | | | GSGGSGGSGGSGGSGGSGGSGGSGGSGGS |
| | | | GGSGGSGGSGGSGAAEGVQIQIIYFNLETV |
| | | | QVTWNASKYSRTNLTFHYRFNGDEAYDQ |
| | | | CTNYLLQEGHTSGCLLDAEQRDDILYFSIR |
| | | | NGTHPVFTASRWMVYYLKPSSPKHVRFSW |
| | | | HQDAVTVTCSDLSYGDLLYEVQYRSPFDT |
| | | | EWQSKQENTCNVTIEGLDAEKCYSFWVRV |
| | | | KAMEDVYGPDTYPSDWSEVTCWQRGEIR |
| | | | DACAETPTPPKPKLSK |
| TSLP | Full Length | 31 | GAAEGVQIQIIYFNLETVQVTWNASKYSRT |
| TRAP | Sequence | | NLTFHYRFNGDEAYDQCTNYLLQEGHTSG |
| 1_Linker- | | | CLLDAEQRDDILYFSIRNGTHPVFTASRWM |
| GS4_ | | | VYYLKPSSPKHVRFSWHQDAVTVTCSDLS |
| Dupilumab- | | | YGDLLYEVQYRSPFDTEWQSKQENTCNVT |
| YTE | | | IEGLDAEKCYSFWVRVKAMEDVYGPDTYP |
| | | | SDWSEVTCWQRGEIRDACAETPTPPKPKLS |
| | | | KGGSGGSGGSGGSGGSGGSGGSGGSGGSG |
| | | | GSGGSGGSGGSGGSGGSGGSGGSGGSGGS |
| | | | GGSESGYAQNGDLEDAELDDYSFSCYSQL |
| | | | EVNGSQHSLTCAFEDPDVNITNLEFEICGAL |
| | | | VEVKCLNFRKLQEIYFIETKKFLLIGKSNIC |
| | | | VKVGEKSLTCKKIDLTTIVKPEAPFDLSVV |
| | | | YREGANDFVVTFNTSHLQKKYVKVLMHD |
| | | | VAYRQEKDENKWTHVNLSSTKLTLLQRKL |
| | | | QPAAMYEIKVRSIPDHYFKGFWSEWSPSY |
| | | | YFRTPEINNSSGEMDGGGGSGGGGSGGGG |
| | | | SGGGGSEVQLVESGGGLEQPGGSLRLSCA |
| | | | GSGFTFRDYAMTWVRQAPGKGLEWVSSIS |
| | | | GSGGNTYYADSVKGRFTISRDNSKNTLYL |
| | | | QMNSLRAEDTAVYYCAKDRLSITIRPRYYG |
| | | | LDVWGQGTTVTVSSASTKGPSVFPLAPCSR |
| | | | STSESTAALGCLVKDYFPEPVTVSWNSGAL |
| | | | TSGVHTFPAVLQSSGLYSLSSVVTVPSSSLG |
| | | | TKTYTCNVDHKPSNTKVDKRVESKYGPPC |
| | | | PPCPAPEFLGGPSVFLFPPKPKDTLYITREPE |
| | | | VTCVVVDVSQEDPEVQFNWYVDGVEVHN |
| | | | AKTKPREEQFNSTYRVVSVLTVLHQDWLN |
| | | | GKEYKCKVSNKGLPSSIEKTISKAKGQPRE |
| | | | PQVYTLPPSQEEMTKNQVSLTCLVKGFYPS |
| | | | DIAVEWESNGQPENNYKTTPPVLDSDGSFF |
| | | | LYSRLTVDKSRWQEGNVFSCSVMHEALHN |
| | | | HYTQKSLSLSLGDIVMTQSPLSLPVTPGEPA |
| | | | SISCRSSQSLLYSIGYNYLDWYLQKSGQSP |
| | | | QLLIYLGSNRASGVPDRFSGSGSGTDFTLKI |
| | | | SRVEAEDVGFYYCMQALQTPYTFGQGTKL |
| | | | EIKRTVAAPSVFIFPPSDEQLKSGTASVVCL |
| | | | LNNFYPREAKVQWKVDNALQSGNSQESVT |
| | | | EQDSKDSTYSLSSTLTLSKADYEKHKVYAC |
| | | | EVTHQGLSSPVTKSFNRGEC |
| | VH Domain | 10 | EVQLVESGGGLEQPGGSLRLSCAGSGFTFR |
| | | | DYAMTWVRQAPGKGLEWVSSISGSGGNT |
| | | | YYADSVKGRFTISRDNSKNTLYLQMNSLR |
| | | | AEDTAVYYCAKDRLSITIRPRYYGLDVWG |
| | | | QGTTVTVS |
| | VL Domain | 15 | DIVMTQSPLSLPVTPGEPASISCRSSQSLLYS |
| | | | IGYNYLDWYLQKSGQSPQLLIYLGSNRASG |
| | | | VPDRFSGSGSGTDFTLKISRVEAEDVGFYY |
| | | | CMQALQTPYTFGQGTKLEIK |
| | First Linker | 6 | GGGGSGGGGSGGGGSGGGGS |
| | Second Linker | 3 | GGSGGSGGSGGSGGSGGSGGSGGSGGSGG |
| | | | SGGSGGSGGSGGSGGSGGSGGSGGSGGSG |
| | | | GS |
| | HCDR1 | 7 | GFTFRDYA |
| | HCDR2 | 8 | ISGSGGNT |
| | HCDR3 | 9 | AKDRLSITIRPRYYGLDV |
| | LCDR1 | 12 | QSLLYSIGYNY |
| | LCDR2 | 13 | LGS |
| | LCDR3 | 14 | MQALQTPYT |
| | TSLP-TRAP | 4 | GAAEGVQIQIIYFNLETVQVTWNASKYSRT |
| | | | NLTFHYRFNGDEAYDQCTNYLLQEGHTSG |
| | | | CLLDAEQRDDILYFSIRNGTHPVFTASRWM |
| | | | VYYLKPSSPKHVRFSWHQDAVTVTCSDLS |
| | | | YGDLLYEVQYRSPFDTEWQSKQENTCNVT |
| | | | IEGLDAEKCYSFWVRVKAMEDVYGPDTYP |
| | | | SDWSEVTCWQRGEIRDACAETPTPPKPKLS |
| | | | KGGSGGSGGSGGSGGSGGSGGSGGSGGSG |
| | | | GSGGSGGSGGSGGSGGSGGSGGSGGSGGS |
| | | | GGSESGYAQNGDLEDAELDDYSFSCYSQL |
| | | | EVNGSQHSLTCAFEDPDVNITNLEFEICGAL |
| | | | VEVKCLNFRKLQEIYFIETKKFLLIGKSNIC |
| | | | VKVGEKSLTCKKIDLTTIVKPEAPFDLSVV |
| | | | YREGANDFVVTFNTSHLQKKYVKVLMHD |
| | | | VAYRQEKDENKWTHVNLSSTKLTLLQRKL |
| | | | QPAAMYEIKVRSIPDHYFKGFWSEWSPSY |
| | | | YFRTPEINNSSGEMD |
| TSLP | Full Length | 32 | ESGYAQNGDLEDAELDDYSFSCYSQLEVN |
| TRAP | Sequence | | GSQHSLTCAFEDPDVNITNLEFEICGALVEV |
| 2_Linker- | | | KCLNFRKLQEIYFIETKKFLLIGKSNICVKV |
| GS4_ | | | GEKSLTCKKIDLTTIVKPEAPFDLSVVYREG |
| Dupilumab- | | | ANDFVVTFNTSHLQKKYVKVLMHDVAYR |
| YTE | | | QEKDENKWTHVNLSSTKLTLLQRKLQPAA |
| | | | MYEIKVRSIPDHYFKGFWSEWSPSYYFRTP |
| | | | EINNSSGEMDGGSGGSGGSGGSGGSGGSG |
| | | | GSGGSGGSGGSGGSGGSGGSGGSGGSGGS |
| | | | GGSGGSGGSGGSGAAEGVQIQIIYFNLETV |
| | | | QVTWNASKYSRTNLTFHYRFNGDEAYDQ |
| | | | CTNYLLQEGHTSGCLLDAEQRDDILYFSIR |
| | | | NGTHPVFTASRWMVYYLKPSSPKHVRFSW |
| | | | HQDAVTVTCSDLSYGDLLYEVQYRSPFDT |
| | | | EWQSKQENTCNVTIEGLDAEKCYSFWVRV |
| | | | KAMEDVYGPDTYPSDWSEVTCWQRGEIR |
| | | | DACAETPTPPKPKLSKGGGGSGGGGSGGG |
| | | | GSGGGGSEVQLVESGGGLEQPGGSLRLSC |
| | | | AGSGFTFRDYAMTWVRQAPGKGLEWVSSI |
| | | | SGSGGNTYYADSVKGRFTISRDNSKNTLYL |
| | | | QMNSLRAEDTAVYYCAKDRLSITIRPRYYG |
| | | | LDVWGQGTTVTVSSASTKGPSVFPLAPCSR |
| | | | STSESTAALGCLVKDYFPEPVTVSWNSGAL |
| | | | TSGVHTFPAVLQSSGLYSLSSVVTVPSSSLG |
| | | | TKTYTCNVDHKPSNTKVDKRVESKYGPPC |
| | | | PPCPAPEFLGGPSVFLFPPKPKDTLYITREPE |
| | | | VTCVVVDVSQEDPEVQFNWYVDGVEVHN |
| | | | AKTKPREEQFNSTYRVVSVLTVLHQDWLN |
| | | | GKEYKCKVSNKGLPSSIEKTISKAKGQPRE |
| | | | PQVYTLPPSQEEMTKNQVSLTCLVKGFYPS |
| | | | DIAVEWESNGQPENNYKTTPPVLDSDGSFF |
| | | | LYSRLTVDKSRWQEGNVFSCSVMHEALHN |
| | | | HYTQKSLSLSLGDIVMTQSPLSLPVTPGEPA |
| | | | SISCRSSQSLLYSIGYNYLDWYLQKSGQSP |
| | | | QLLIYLGSNRASGVPDRFSGSGSGTDFTLKI |
| | | | SRVEAEDVGFYYCMQALQTPYTFGQGTKL |
| | | | EIKRTVAAPSVFIFPPSDEQLKSGTASVVCL |
| | | | LNNFYPREAKVQWKVDNALQSGNSQESVT |
| | | | EQDSKDSTYSLSSTLTLSKADYEKHKVYAC |
| | | | EVTHQGLSSPVTKSFNRGEC |
| | VH Domain | 10 | EVQLVESGGGLEQPGGSLRLSCAGSGFTFR |
| | | | DYAMTWVRQAPGKGLEWVSSISGSGGNT |
| | | | YYADSVKGRFTISRDNSKNTLYLQMNSLR |
| | | | AEDTAVYYCAKDRLSITIRPRYYGLDVWG |
| | | | QGTTVTVS |
| | VL Domain | 15 | DIVMTQSPLSLPVTPGEPASISCRSSQSLLYS |
| | | | IGYNYLDWYLQKSGQSPQLLIYLGSNRASG |
| | | | VPDRFSGSGSGTDFTLKISRVEAEDVGFYY |
| | | | CMQALQTPYTFGQGTKLEIK |
| | First Linker | 6 | GGGGSGGGGSGGGGSGGGGS |
| | Second Linker | 3 | GGSGGSGGSGGSGGSGGSGGSGGSGGSGG |
| | | | SGGSGGSGGSGGSGGSGGSGGSGGSGGSG |
| | | | GS |
| | HCDR1 | 7 | GFTFRDYA |
| | HCDR2 | 8 | ISGSGGNT |
| | HCDR3 | 9 | AKDRLSITIRPRYYGLDV |
| | LCDR1 | 12 | QSLLYSIGYNY |
| | LCDR2 | 13 | LGS |
| | LCDR3 | 14 | MQALQTPYT |
| | TSLP-TRAP | 5 | ESGYAQNGDLEDAELDDYSFSCYSQLEVN |
| | | | GSQHSLTCAFEDPDVNITNLEFEICGALVEV |
| | | | KCLNFRKLQEIYFIETKKFLLIGKSNICVKV |
| | | | GEKSLTCKKIDLTTIVKPEAPFDLSVVYREG |
| | | | ANDFVVTFNTSHLQKKYVKVLMHDVAYR |
| | | | QEKDENKWTHVNLSSTKLTLLQRKLQPAA |
| | | | MYEIKVRSIPDHYFKGFWSEWSPSYYFRTP |
| | | | EINNSSGEMDGGSGGSGGSGGSGGSGGSG |
| | | | GSGGSGGSGGSGGSGGSGGSGGSGGSGGS |
| | | | GGSGGSGGSGGSGAAEGVQIQIIYFNLETV |
| | | | QVTWNASKYSRTNLTFHYRFNGDEAYDQ |
| | | | CTNYLLQEGHTSGCLLDAEQRDDILYFSIR |
| | | | NGTHPVFTASRWMVYYLKPSSPKHVRFSW |
| | | | HQDAVTVTCSDLSYGDLLYEVQYRSPFDT |
| | | | EWQSKQENTCNVTIEGLDAEKCYSFWVRV |
| | | | KAMEDVYGPDTYPSDWSEVTCWQRGEIR |
| | | | DACAETPTPPKPKLSK |
| Dupilumab- | Full Length | 33 | EVQLVESGGGLEQPGGSLRLSCAGSGFTFR |
| YTE_Linker- | Sequence | | DYAMTWVRQAPGKGLEWVSSISGSGGNT |
| GS4_TSLP | | | YYADSVKGRFTISRDNSKNTLYLQMNSLR |
| TRAP 1 | | | AEDTAVYYCAKDRLSITIRPRYYGLDVWG |
| | | | QGTTVTVSSASTKGPSVFPLAPCSRSTSEST |
| | | | AALGCLVKDYFPEPVTVSWNSGALTSGVH |
| | | | TFPAVLQSSGLYSLSSVVTVPSSSLGTKTYT |
| | | | CNVDHKPSNTKVDKRVESKYGPPCPPCPA |
| | | | PEFLGGPSVFLFPPKPKDTLYITREPEVTCV |
| | | | VVDVSQEDPEVQFNWYVDGVEVHNAKTK |
| | | | PREEQFNSTYRVVSVLTVLHQDWLNGKEY |
| | | | KCKVSNKGLPSSIEKTISKAKGQPREPQVY |
| | | | TLPPSQEEMTKNQVSLTCLVKGFYPSDIAV |
| | | | EWESNGQPENNYKTTPPVLDSDGSFFLYSR |
| | | | LTVDKSRWQEGNVFSCSVMHEALHNHYT |
| | | | QKSLSLSLGGGGGSGGGGSGGGGSGGGGS |
| | | | GAAEGVQIQIIYFNLETVQVTWNASKYSRT |
| | | | NLTFHYRFNGDEAYDQCTNYLLQEGHTSG |
| | | | CLLDAEQRDDILYFSIRNGTHPVFTASRWM |
| | | | VYYLKPSSPKHVRFSWHQDAVTVTCSDLS |
| | | | YGDLLYEVQYRSPFDTEWQSKQENTCNVT |
| | | | IEGLDAEKCYSFWVRVKAMEDVYGPDTYP |
| | | | SDWSEVTCWQRGEIRDACAETPTPPKPKLS |
| | | | KGGSGGSGGSGGSGGSGGSGGSGGSGGSG |
| | | | GSGGSGGSGGSGGSGGSGGSGGSGGSGGS |
| | | | GGSESGYAQNGDLEDAELDDYSFSCYSQL |
| | | | EVNGSQHSLTCAFEDPDVNITNLEFEICGAL |
| | | | VEVKCLNFRKLQEIYFIETKKFLLIGKSNIC |
| | | | VKVGEKSLTCKKIDLTTIVKPEAPFDLSVV |
| | | | YREGANDFVVTFNTSHLQKKYVKVLMHD |
| | | | VAYRQEKDENKWTHVNLSSTKLTLLQRKL |
| | | | QPAAMYEIKVRSIPDHYFKGFWSEWSPSY |
| | | | YFRTPEINNSSGEMDDIVMTQSPLSLPVTPG |
| | | | EPASISCRSSQSLLYSIGYNYLDWYLQKSG |
| | | | QSPQLLIYLGSNRASGVPDRFSGSGSGTDFT |
| | | | LKISRVEAEDVGFYYCMQALQTPYTFGQG |
| | | | TKLEIKRTVAAPSVFIFPPSDEQLKSGTASV |
| | | | VCLLNNFYPREAKVQWKVDNALQSGNSQ |
| | | | ESVTEQDSKDSTYSLSSTLTLSKADYEKHK |
| | | | VYACEVTHQGLSSPVTKSFNRGEC |
| | VH Domain | 10 | EVQLVESGGGLEQPGGSLRLSCAGSGFTFR |
| | | | DYAMTWVRQAPGKGLEWVSSISGSGGNT |
| | | | YYADSVKGRFTISRDNSKNTLYLQMNSLR |
| | | | AEDTAVYYCAKDRLSITIRPRYYGLDVWG |
| | | | QGTTVTVS |
| | VL Domain | 15 | DIVMTQSPLSLPVTPGEPASISCRSSQSLLYS |
| | | | IGYNYLDWYLQKSGQSPQLLIYLGSNRASG |
| | | | VPDRFSGSGSGTDFTLKISRVEAEDVGFYY |
| | | | CMQALQTPYTFGQGTKLEIK |
| | First Linker | 6 | GGGGSGGGGSGGGGSGGGGS |
| | Second Linker | 3 | GGSGGSGGSGGSGGSGGSGGSGGSGGSGG |
| | | | SGGSGGSGGSGGSGGSGGSGGSGGSGGSG |
| | | | GS |
| | HCDR1 | 7 | GFTFRDYA |
| | HCDR2 | 8 | ISGSGGNT |
| | HCDR3 | 9 | AKDRLSITIRPRYYGLDV |
| | LCDR1 | 12 | QSLLYSIGYNY |
| | LCDR2 | 13 | LGS |
| | LCDR3 | 14 | MQALQTPYT |
| | TSLP-TRAP | 4 | GAAEGVQIQIIYFNLETVQVTWNASKYSRT |
| | | | NLTFHYRFNGDEAYDQCTNYLLQEGHTSG |
| | | | CLLDAEQRDDILYFSIRNGTHPVFTASRWM |
| | | | VYYLKPSSPKHVRFSWHQDAVTVTCSDLS |
| | | | YGDLLYEVQYRSPFDTEWQSKQENTCNVT |
| | | | IEGLDAEKCYSFWVRVKAMEDVYGPDTYP |
| | | | SDWSEVTCWQRGEIRDACAETPTPPKPKLS |
| | | | KGGSGGSGGSGGSGGSGGSGGSGGSGGSG |
| | | | GSGGSGGSGGSGGSGGSGGSGGSGGSGGS |
| | | | GGSESGYAQNGDLEDAELDDYSFSCYSQL |
| | | | EVNGSQHSLTCAFEDPDVNITNLEFEICGAL |
| | | | VEVKCLNFRKLQEIYFIETKKFLLIGKSNIC |
| | | | VKVGEKSLTCKKIDLTTIVKPEAPFDLSVV |
| | | | YREGANDFVVTFNTSHLQKKYVKVLMHD |
| | | | VAYRQEKDENKWTHVNLSSTKLTLLQRKL |
| | | | QPAAMYEIKVRSIPDHYFKGFWSEWSPSY |
| | | | YFRTPEINNSSGEMD |
| Dupilumab- | Full Length | 34 | EVQLVESGGGLEQPGGSLRLSCAGSGFTFR |
| YTE_Linker- | Sequence | | DYAMTWVRQAPGKGLEWVSSISGSGGNT |
| GS4_TSLP | | | YYADSVKGRFTISRDNSKNTLYLQMNSLR |
| TRAP 2 | | | AEDTAVYYCAKDRLSITIRPRYYGLDVWG |
| | | | QGTTVTVSSASTKGPSVFPLAPCSRSTSEST |
| | | | AALGCLVKDYFPEPVTVSWNSGALTSGVH |
| | | | TFPAVLQSSGLYSLSSVVTVPSSSLGTKTYT |
| | | | CNVDHKPSNTKVDKRVESKYGPPCPPCPA |
| | | | PEFLGGPSVFLFPPKPKDTLYITREPEVTCV |
| | | | VVDVSQEDPEVQFNWYVDGVEVHNAKTK |
| | | | PREEQFNSTYRVVSVLTVLHQDWLNGKEY |
| | | | KCKVSNKGLPSSIEKTISKAKGQPREPQVY |
| | | | TLPPSQEEMTKNQVSLTCLVKGFYPSDIAV |
| | | | EWESNGQPENNYKTTPPVLDSDGSFFLYSR |
| | | | LTVDKSRWQEGNVFSCSVMHEALHNHYT |
| | | | QKSLSLSLGGGGGSGGGGSGGGGSGGGGS |
| | | | ESGYAQNGDLEDAELDDYSFSCYSQLEVN |
| | | | GSQHSLTCAFEDPDVNITNLEFEICGALVEV |
| | | | KCLNFRKLQEIYFIETKKFLLIGKSNICVKV |
| | | | GEKSLTCKKIDLTTIVKPEAPFDLSVVYREG |
| | | | ANDFVVTFNTSHLQKKYVKVLMHDVAYR |
| | | | QEKDENKWTHVNLSSTKLTLLQRKLQPAA |
| | | | MYEIKVRSIPDHYFKGFWSEWSPSYYFRTP |
| | | | EINNSSGEMDGGSGGSGGSGGSGGSGGSG |
| | | | GSGGSGGSGGSGGSGGSGGSGGSGGSGGS |
| | | | GGSGGSGGSGGSGAAEGVQIQIIYFNLETV |
| | | | QVTWNASKYSRTNLTFHYRFNGDEAYDQ |
| | | | CTNYLLQEGHTSGCLLDAEQRDDILYFSIR |
| | | | NGTHPVFTASRWMVYYLKPSSPKHVRFSW |
| | | | HQDAVTVTCSDLSYGDLLYEVQYRSPFDT |
| | | | EWQSKQENTCNVTIEGLDAEKCYSFWVRV |
| | | | KAMEDVYGPDTYPSDWSEVTCWQRGEIR |
| | | | DACAETPTPPKPKLSKDIVMTQSPLSLPVTP |
| | | | GEPASISCRSSQSLLYSIGYNYLDWYLQKS |
| | | | GQSPQLLIYLGSNRASGVPDRFSGSGSGTD |
| | | | FTLKISRVEAEDVGFYYCMQALQTPYTFGQ |
| | | | GTKLEIKRTVAAPSVFIFPPSDEQLKSGTAS |
| | | | VVCLLNNFYPREAKVQWKVDNALQSGNS |
| | | | QESVTEQDSKDSTYSLSSTLTLSKADYEKH |
| | | | KVYACEVTHQGLSSPVTKSFNRGEC |
| | VH Domain | 10 | EVQLVESGGGLEQPGGSLRLSCAGSGFTFR |
| | | | DYAMTWVRQAPGKGLEWVSSISGSGGNT |
| | | | YYADSVKGRFTISRDNSKNTLYLQMNSLR |
| | | | AEDTAVYYCAKDRLSITIRPRYYGLDVWG |
| | | | QGTTVTVS |
| | VL Domain | 15 | DIVMTQSPLSLPVTPGEPASISCRSSQSLLYS |
| | | | IGYNYLDWYLQKSGQSPQLLIYLGSNRASG |
| | | | VPDRFSGSGSGTDFTLKISRVEAEDVGFYY |
| | | | CMQALQTPYTFGQGTKLEIK |
| | First Linker | 6 | GGGGSGGGGSGGGGSGGGGS |
| | Second Linker | 3 | GGSGGSGGSGGSGGSGGSGGSGGSGGSGG |
| | | | SGGSGGSGGSGGSGGSGGSGGSGGSGGSG |
| | | | GS |
| | HCDR1 | 7 | GFTFRDYA |
| | HCDR2 | 8 | ISGSGGNT |
| | HCDR3 | 9 | AKDRLSITIRPRYYGLDV |
| | LCDR1 | 12 | QSLLYSIGYNY |
| | LCDR2 | 13 | LGS |
| | LCDR3 | 14 | MQALQTPYT |
| | TSLP-TRAP | 5 | ESGYAQNGDLEDAELDDYSFSCYSQLEVN |
| | | | GSQHSLTCAFEDPDVNITNLEFEICGALVEV |
| | | | KCLNFRKLQEIYFIETKKFLLIGKSNICVKV |
| | | | GEKSLTCKKIDLTTIVKPEAPFDLSVVYREG |
| | | | ANDFVVTFNTSHLQKKYVKVLMHDVAYR |
| | | | QEKDENKWTHVNLSSTKLTLLQRKLQPAA |
| | | | MYEIKVRSIPDHYFKGFWSEWSPSYYFRTP |
| | | | EINNSSGEMDGGSGGSGGSGGSGGSGGSG |
| | | | GSGGSGGSGGSGGSGGSGGSGGSGGSGGS |
| | | | GGSGGSGGSGGSGAAEGVQIQIIYFNLETV |
| | | | QVTWNASKYSRTNLTFHYRFNGDEAYDQ |
| | | | CTNYLLQEGHTSGCLLDAEQRDDILYFSIR |
| | | | NGTHPVFTASRWMVYYLKPSSPKHVRFSW |
| | | | HQDAVTVTCSDLSYGDLLYEVQYRSPFDT |
| | | | EWQSKQENTCNVTIEGLDAEKCYSFWVRV |
| | | | KAMEDVYGPDTYPSDWSEVTCWQRGEIR |
| | | | DACAETPTPPKPKLSK |
| Light chain | Full Length | 35 | EVQLVESGGGLEQPGGSLRLSCAGSGFTFR |
| N-terminal | Sequence | | DYAMTWVRQAPGKGLEWVSSISGSGGNT |
| TRAP: | | | YYADSVKGRFTISRDNSKNTLYLQMNSLR |
| TSLP | | | AEDTAVYYCAKDRLSITIRPRYYGLDVWG |
| TRAP | | | QGTTVTVSSASTKGPSVFPLAPCSRSTSEST |
| 2_Linker- | | | AALGCLVKDYFPEPVTVSWNSGALTSGVH |
| GS4_ | | | TFPAVLQSSGLYSLSSVVTVPSSSLGTKTYT |
| Dupilumab- | | | CNVDHKPSNTKVDKRVESKYGPPCPPCPA |
| YTE [2 x | | | PEFLGGPSVFLFPPKPKDTLYITREPEVTCV |
| TRAP 2] | | | VVDVSQEDPEVQFNWYVDGVEVHNAKTK |
| | | | PREEQFNSTYRVVSVLTVLHQDWLNGKEY |
| | | | KCKVSNKGLPSSIEKTISKAKGQPREPQVY |
| | | | TLPPSQEEMTKNQVSLTCLVKGFYPSDIAV |
| | | | EWESNGQPENNYKTTPPVLDSDGSFFLYSR |
| | | | LTVDKSRWQEGNVFSCSVMHEALHNHYT |
| | | | QKSLSLSLGESGYAQNGDLEDAELDDYSFS |
| | | | CYSQLEVNGSQHSLTCAFEDPDVNITNLEF |
| | | | EICGALVEVKCLNFRKLQEIYFIETKKFLLI |
| | | | GKSNICVKVGEKSLTCKKIDLTTIVKPEAPF |
| | | | DLSVVYREGANDFVVTFNTSHLQKKYVKV |
| | | | LMHDVAYRQEKDENKWTHVNLSSTKLTL |
| | | | LQRKLQPAAMYEIKVRSIPDHYFKGFWSE |
| | | | WSPSYYFRTPEINNSSGEMDGGSGGSGGSG |
| | | | GSGGSGGSGGSGGSGGSGGSGGSGGSGGS |
| | | | GGSGGSGGSGGSGGSGGSGGSGAAEGVQI |
| | | | QIIYFNLETVQVTWNASKYSRTNLTFHYRF |
| | | | NGDEAYDQCTNYLLQEGHTSGCLLDAEQR |
| | | | DDILYFSIRNGTHPVFTASRWMVYYLKPSS |
| | | | PKHVRFSWHQDAVTVTCSDLSYGDLLYEV |
| | | | QYRSPFDTEWQSKQENTCNVTIEGLDAEK |
| | | | CYSFWVRVKAMEDVYGPDTYPSDWSEVT |
| | | | CWQRGEIRDACAETPTPPKPKLSKGGGGSG |
| | | | GGGSGGGGSGGGGSDIVMTQSPLSLPVTPG |
| | | | EPASISCRSSQSLLYSIGYNYLDWYLQKSG |
| | | | QSPQLLIYLGSNRASGVPDRFSGSGSGTDFT |
| | | | LKISRVEAEDVGFYYCMQALQTPYTFGQG |
| | | | TKLEIKRTVAAPSVFIFPPSDEQLKSGTASV |
| | | | VCLLNNFYPREAKVQWKVDNALQSGNSQ |
| | | | ESVTEQDSKDSTYSLSSTLTLSKADYEKHK |
| | | | VYACEVTHQGLSSPVTKSFNRGEC |
| | VH Domain | 10 | EVQLVESGGGLEQPGGSLRLSCAGSGFTFR |
| | | | DYAMTWVRQAPGKGLEWVSSISGSGGNT |
| | | | YYADSVKGRFTISRDNSKNTLYLQMNSLR |
| | | | AEDTAVYYCAKDRLSITIRPRYYGLDVWG |
| | | | QGTTVTVS |
| | VL Domain | 15 | DIVMTQSPLSLPVTPGEPASISCRSSQSLLYS |
| | | | IGYNYLDWYLQKSGQSPQLLIYLGSNRASG |
| | | | VPDRFSGSGSGTDFTLKISRVEAEDVGFYY |
| | | | CMQALQTPYTFGQGTKLEIK |
| | First Linker | 6 | GGGGSGGGGSGGGGSGGGGS |
| | Second Linker | 3 | GGSGGSGGSGGSGGSGGSGGSGGSGGSGG |
| | | | SGGSGGSGGSGGSGGSGGSGGSGGSGGSG |
| | | | GS |
| | HCDR1 | 7 | GFTFRDYA |
| | HCDR2 | 8 | ISGSGGNT |
| | HCDR3 | 9 | AKDRLSITIRPRYYGLDV |
| | LCDR1 | 12 | QSLLYSIGYNY |
| | LCDR2 | 13 | LGS |
| | LCDR3 | 14 | MQALQTPYT |
| | TSLP-TRAP | 5 | ESGYAQNGDLEDAELDDYSFSCYSQLEVN |
| | | | GSQHSLTCAFEDPDVNITNLEFEICGALVEV |
| | | | KCLNFRKLQEIYFIETKKFLLIGKSNICVKV |
| | | | GEKSLTCKKIDLTTIVKPEAPFDLSVVYREG |
| | | | ANDFVVTFNTSHLQKKYVKVLMHDVAYR |
| | | | QEKDENKWTHVNLSSTKLTLLQRKLQPAA |
| | | | MYEIKVRSIPDHYFKGFWSEWSPSYYFRTP |
| | | | EINNSSGEMDGGSGGSGGSGGSGGSGGSG |
| | | | GSGGSGGSGGSGGSGGSGGSGGSGGSGGS |
| | | | GGSGGSGGSGGSGAAEGVQIQIIYFNLETV |
| | | | QVTWNASKYSRTNLTFHYRFNGDEAYDQ |
| | | | CTNYLLQEGHTSGCLLDAEQRDDILYFSIR |
| | | | NGTHPVFTASRWMVYYLKPSSPKHVRFSW |
| | | | HQDAVTVTCSDLSYGDLLYEVQYRSPFDT |
| | | | EWQSKQENTCNVTIEGLDAEKCYSFWVRV |
| | | | KAMEDVYGPDTYPSDWSEVTCWQRGEIR |
| | | | DACAETPTPPKPKLSK |
| Light chain | Full Length | 36 | EVQLVESGGGLEQPGGSLRLSCAGSGFTFR |
| C-terminal | Sequence | | DYAMTWVRQAPGKGLEWVSSISGSGGNT |
| TRAP: | | | YYADSVKGRFTISRDNSKNTLYLQMNSLR |
| Dupilumab- | | | AEDTAVYYCAKDRLSITIRPRYYGLDVWG |
| YTE_Linker- | | | QGTTVTVSSASTKGPSVFPLAPCSRSTSEST |
| GS4_TSLP | | | AALGCLVKDYFPEPVTVSWNSGALTSGVH |
| TRAP 2 [2 | | | TFPAVLQSSGLYSLSSVVTVPSSSLGTKTYT |
| x TRAP 2] | | | CNVDHKPSNTKVDKRVESKYGPPCPPCPA |
| | | | PEFLGGPSVFLFPPKPKDTLYITREPEVTCV |
| | | | VVDVSQEDPEVQFNWYVDGVEVHNAKTK |
| | | | PREEQFNSTYRVVSVLTVLHQDWLNGKEY |
| | | | KCKVSNKGLPSSIEKTISKAKGQPREPQVY |
| | | | TLPPSQEEMTKNQVSLTCLVKGFYPSDIAV |
| | | | EWESNGQPENNYKTTPPVLDSDGSFFLYSR |
| | | | LTVDKSRWQEGNVFSCSVMHEALHNHYT |
| | | | QKSLSLSLGDIVMTQSPLSLPVTPGEPASIS |
| | | | CRSSQSLLYSIGYNYLDWYLQKSGQSPQLL |
| | | | IYLGSNRASGVPDRFSGSGSGTDFTLKISRV |
| | | | EAEDVGFYYCMQALQTPYTFGQGTKLEIK |
| | | | RTVAAPSVFIFPPSDEQLKSGTASVVCLLN |
| | | | NFYPREAKVQWKVDNALQSGNSQESVTEQ |
| | | | DSKDSTYSLSSTLTLSKADYEKHKVYACEV |
| | | | THQGLSSPVTKSFNRGECGGGGSGGGGSG |
| | | | GGGSGGGGSESGYAQNGDLEDAELDDYSF |
| | | | SCYSQLEVNGSQHSLTCAFEDPDVNITNLE |
| | | | FEICGALVEVKCLNFRKLQEIYFIETKKFLLI |
| | | | GKSNICVKVGEKSLTCKKIDLTTIVKPEAPF |
| | | | DLSVVYREGANDFVVTFNTSHLQKKYVKV |
| | | | LMHDVAYRQEKDENKWTHVNLSSTKLTL |
| | | | LQRKLQPAAMYEIKVRSIPDHYFKGFWSE |
| | | | WSPSYYFRTPEINNSSGEMDGGSGGSGGSG |
| | | | GSGGSGGSGGSGGSGGSGGSGGSGGSGGS |
| | | | GGSGGSGGSGGSGGSGGSGGSGAAEGVQI |
| | | | QIIYFNLETVQVTWNASKYSRTNLTFHYRF |
| | | | NGDEAYDQCTNYLLQEGHTSGCLLDAEQR |
| | | | DDILYFSIRNGTHPVFTASRWMVYYLKPSS |
| | | | PKHVRFSWHQDAVTVTCSDLSYGDLLYEV |
| | | | QYRSPFDTEWQSKQENTCNVTIEGLDAEK |
| | | | CYSFWVRVKAMEDVYGPDTYPSDWSEVT |
| | | | CWQRGEIRDACAETPTPPKPKLSK |
| | VH Domain | 10 | EVQLVESGGGLEQPGGSLRLSCAGSGFTFR |
| | | | DYAMTWVRQAPGKGLEWVSSISGSGGNT |
| | | | YYADSVKGRFTISRDNSKNTLYLQMNSLR |
| | | | AEDTAVYYCAKDRLSITIRPRYYGLDVWG |
| | | | QGTTVTVS |
| | VL Domain | 15 | DIVMTQSPLSLPVTPGEPASISCRSSQSLLYS |
| | | | IGYNYLDWYLQKSGQSPQLLIYLGSNRASG |
| | | | VPDRFSGSGSGTDFTLKISRVEAEDVGFYY |
| | | | CMQALQTPYTFGQGTKLEIK |
| | First Linker | 6 | GGGGSGGGGSGGGGSGGGGS |
| | Second Linker | 3 | GGSGGSGGSGGSGGSGGSGGSGGSGGSGG |
| | | | SGGSGGSGGSGGSGGSGGSGGSGGSGGSG |
| | | | GS |
| | HCDR1 | 7 | GFTFRDYA |
| | HCDR2 | 8 | ISGSGGNT |
| | HCDR3 | 9 | AKDRLSITIRPRYYGLDV |
| | LCDR1 | 12 | QSLLYSIGYNY |
| | LCDR2 | 13 | LGS |
| | LCDR3 | 14 | MQALQTPYT |
| | TSLP-TRAP | 5 | ESGYAQNGDLEDAELDDYSFSCYSQLEVN |
| | | | GSQHSLTCAFEDPDVNITNLEFEICGALVEV |
| | | | KCLNFRKLQEIYFIETKKFLLIGKSNICVKV |
| | | | GEKSLTCKKIDLTTIVKPEAPFDLSVVYREG |
| | | | ANDFVVTFNTSHLQKKYVKVLMHDVAYR |
| | | | QEKDENKWTHVNLSSTKLTLLQRKLQPAA |
| | | | MYEIKVRSIPDHYFKGFWSEWSPSYYFRTP |
| | | | EINNSSGEMDGGSGGSGGSGGSGGSGGSG |
| | | | GSGGSGGSGGSGGSGGSGGSGGSGGSGGS |
| | | | GGSGGSGGSGGSGAAEGVQIQIIYFNLETV |
| | | | QVTWNASKYSRTNLTFHYRFNGDEAYDQ |
| | | | CTNYLLQEGHTSGCLLDAEQRDDILYFSIR |
| | | | NGTHPVFTASRWMVYYLKPSSPKHVRFSW |
| | | | HQDAVTVTCSDLSYGDLLYEVQYRSPFDT |
| | | | EWQSKQENTCNVTIEGLDAEKCYSFWVRV |
| | | | KAMEDVYGPDTYPSDWSEVTCWQRGEIR |
| | | | DACAETPTPPKPKLSK |
| Heavy and | Full Length | 37 | ESGYAQNGDLEDAELDDYSFSCYSQLEVN |
| Light chain | Sequence | | GSQHSLTCAFEDPDVNITNLEFEICGALVEV |
| N-terminal | | | KCLNFRKLQEIYFIETKKFLLIGKSNICVKV |
| TRAP: | | | GEKSLTCKKIDLTTIVKPEAPFDLSVVYREG |
| TSLP | | | ANDFVVTFNTSHLQKKYVKVLMHDVAYR |
| TRAP | | | QEKDENKWTHVNLSSTKLTLLQRKLQPAA |
| 2_Linker- | | | MYEIKVRSIPDHYFKGFWSEWSPSYYFRTP |
| GS4_ | | | EINNSSGEMDGGSGGSGGSGGSGGSGGSG |
| Dupilumab- | | | GSGGSGGSGGSGGSGGSGGSGGSGGSGGS |
| YTE [4 x | | | GGSGGSGGSGGSGAAEGVQIQIIYFNLETV |
| TRAP 2] | | | QVTWNASKYSRTNLTFHYRFNGDEAYDQ |
| | | | CTNYLLQEGHTSGCLLDAEQRDDILYFSIR |
| | | | NGTHPVFTASRWMVYYLKPSSPKHVRFSW |
| | | | HQDAVTVTCSDLSYGDLLYEVQYRSPFDT |
| | | | EWQSKQENTCNVTIEGLDAEKCYSFWVRV |
| | | | KAMEDVYGPDTYPSDWSEVTCWQRGEIR |
| | | | DACAETPTPPKPKLSKGGGGSGGGGSGGG |
| | | | GSGGGGSEVQLVESGGGLEQPGGSLRLSC |
| | | | AGSGFTFRDYAMTWVRQAPGKGLEWVSSI |
| | | | SGSGGNTYYADSVKGRFTISRDNSKNTLYL |
| | | | QMNSLRAEDTAVYYCAKDRLSITIRPRYYG |
| | | | LDVWGQGTTVTVSSASTKGPSVFPLAPCSR |
| | | | STSESTAALGCLVKDYFPEPVTVSWNSGAL |
| | | | TSGVHTFPAVLQSSGLYSLSSVVTVPSSSLG |
| | | | TKTYTCNVDHKPSNTKVDKRVESKYGPPC |
| | | | PPCPAPEFLGGPSVFLFPPKPKDTLYITREPE |
| | | | VTCVVVDVSQEDPEVQFNWYVDGVEVHN |
| | | | AKTKPREEQFNSTYRVVSVLTVLHQDWLN |
| | | | GKEYKCKVSNKGLPSSIEKTISKAKGQPRE |
| | | | PQVYTLPPSQEEMTKNQVSLTCLVKGFYPS |
| | | | DIAVEWESNGQPENNYKTTPPVLDSDGSFF |
| | | | LYSRLTVDKSRWQEGNVFSCSVMHEALHN |
| | | | HYTQKSLSLSLGESGYAQNGDLEDAELDD |
| | | | YSFSCYSQLEVNGSQHSLTCAFEDPDVNIT |
| | | | NLEFEICGALVEVKCLNFRKLQEIYFIETKK |
| | | | FLLIGKSNICVKVGEKSLTCKKIDLTTIVKP |
| | | | EAPFDLSVVYREGANDFVVTFNTSHLQKK |
| | | | YVKVLMHDVAYRQEKDENKWTHVNLSST |
| | | | KLTLLQRKLQPAAMYEIKVRSIPDHYFKGF |
| | | | WSEWSPSYYFRTPEINNSSGEMDGGSGGS |
| | | | GGSGGSGGSGGSGGSGGSGGSGGSGGSGG |
| | | | SGGSGGSGGSGGSGGSGGSGGSGGSGAAE |
| | | | GVQIQIIYFNLETVQVTWNASKYSRTNLTF |
| | | | HYRFNGDEAYDQCTNYLLQEGHTSGCLLD |
| | | | AEQRDDILYFSIRNGTHPVFTASRWMVYYL |
| | | | KPSSPKHVRFSWHQDAVTVTCSDLSYGDL |
| | | | LYEVQYRSPFDTEWQSKQENTCNVTIEGLD |
| | | | AEKCYSFWVRVKAMEDVYGPDTYPSDWS |
| | | | EVTCWQRGEIRDACAETPTPPKPKLSKGGG |
| | | | GSGGGGSGGGGSGGGGSDIVMTQSPLSLP |
| | | | VTPGEPASISCRSSQSLLYSIGYNYLDWYLQ |
| | | | KSGQSPQLLIYLGSNRASGVPDRFSGSGSG |
| | | | TDFTLKISRVEAEDVGFYYCMQALQTPYTF |
| | | | GQGTKLEIKRTVAAPSVFIFPPSDEQLKSGT |
| | | | ASVVCLLNNFYPREAKVQWKVDNALQSG |
| | | | NSQESVTEQDSKDSTYSLSSTLTLSKADYE |
| | | | KHKVYACEVTHQGLSSPVTKSFNRGEC |
| | VH Domain | 10 | EVQLVESGGGLEQPGGSLRLSCAGSGFTFR |
| | | | DYAMTWVRQAPGKGLEWVSSISGSGGNT |
| | | | YYADSVKGRFTISRDNSKNTLYLQMNSLR |
| | | | AEDTAVYYCAKDRLSITIRPRYYGLDVWG |
| | | | QGTTVTVS |
| | VL Domain | 15 | DIVMTQSPLSLPVTPGEPASISCRSSQSLLYS |
| | | | IGYNYLDWYLQKSGQSPQLLIYLGSNRASG |
| | | | VPDRFSGSGSGTDFTLKISRVEAEDVGFYY |
| | | | CMQALQTPYTFGQGTKLEIK |
| | First Linker | 6 | GGGGSGGGGSGGGGSGGGGS |
| | Second Linker | 3 | GGSGGSGGSGGSGGSGGSGGSGGSGGSGG |
| | | | SGGSGGSGGSGGSGGSGGSGGSGGSGGSG |
| | | | GS |
| | HCDR1 | 7 | GFTFRDYA |
| | HCDR2 | 8 | ISGSGGNT |
| | HCDR3 | 9 | AKDRLSITIRPRYYGLDV |
| | LCDR1 | 12 | QSLLYSIGYNY |
| | LCDR2 | 13 | LGS |
| | LCDR3 | 14 | MQALQTPYT |
| | TSLP-TRAP | 5 | ESGYAQNGDLEDAELDDYSFSCYSQLEVN |
| | | | GSQHSLTCAFEDPDVNITNLEFEICGALVEV |
| | | | KCLNFRKLQEIYFIETKKFLLIGKSNICVKV |
| | | | GEKSLTCKKIDLTTIVKPEAPFDLSVVYREG |
| | | | ANDFVVTFNTSHLQKKYVKVLMHDVAYR |
| | | | QEKDENKWTHVNLSSTKLTLLQRKLQPAA |
| | | | MYEIKVRSIPDHYFKGFWSEWSPSYYFRTP |
| | | | EINNSSGEMDGGSGGSGGSGGSGGSGGSG |
| | | | GSGGSGGSGGSGGSGGSGGSGGSGGSGGS |
| | | | GGSGGSGGSGGSGAAEGVQIQIIYFNLETV |
| | | | QVTWNASKYSRTNLTFHYRFNGDEAYDQ |
| | | | CTNYLLQEGHTSGCLLDAEQRDDILYFSIR |
| | | | NGTHPVFTASRWMVYYLKPSSPKHVRFSW |
| | | | HQDAVTVTCSDLSYGDLLYEVQYRSPFDT |
| | | | EWQSKQENTCNVTIEGLDAEKCYSFWVRV |
| | | | KAMEDVYGPDTYPSDWSEVTCWQRGEIR |
| | | | DACAETPTPPKPKLSK |
| Heavy and | Full Length | 38 | EVQLVESGGGLEQPGGSLRLSCAGSGFTFR |
| Light chain | Sequence | | DYAMTWVRQAPGKGLEWVSSISGSGGNT |
| C-terminal | | | YYADSVKGRFTISRDNSKNTLYLQMNSLR |
| TRAP: | | | AEDTAVYYCAKDRLSITIRPRYYGLDVWG |
| Dupilumab- | | | QGTTVTVSSASTKGPSVFPLAPCSRSTSEST |
| YTE_Linker- | | | AALGCLVKDYFPEPVTVSWNSGALTSGVH |
| GS4_TSLP | | | TFPAVLQSSGLYSLSSVVTVPSSSLGTKTYT |
| TRAP 2 [4 | | | CNVDHKPSNTKVDKRVESKYGPPCPPCPA |
| x TRAP 2] | | | PEFLGGPSVFLFPPKPKDTLYITREPEVTCV |
| | | | VVDVSQEDPEVQFNWYVDGVEVHNAKTK |
| | | | PREEQFNSTYRVVSVLTVLHQDWLNGKEY |
| | | | KCKVSNKGLPSSIEKTISKAKGQPREPQVY |
| | | | TLPPSQEEMTKNQVSLTCLVKGFYPSDIAV |
| | | | EWESNGQPENNYKTTPPVLDSDGSFFLYSR |
| | | | LTVDKSRWQEGNVFSCSVMHEALHNHYT |
| | | | QKSLSLSLGGGGGSGGGGSGGGGSGGGGS |
| | | | ESGYAQNGDLEDAELDDYSFSCYSQLEVN |
| | | | GSQHSLTCAFEDPDVNITNLEFEICGALVEV |
| | | | KCLNFRKLQEIYFIETKKFLLIGKSNICVKV |
| | | | GEKSLTCKKIDLTTIVKPEAPFDLSVVYREG |
| | | | ANDFVVTFNTSHLQKKYVKVLMHDVAYR |
| | | | QEKDENKWTHVNLSSTKLTLLQRKLQPAA |
| | | | MYEIKVRSIPDHYFKGFWSEWSPSYYFRTP |
| | | | EINNSSGEMDGGSGGSGGSGGSGGSGGSG |
| | | | GSGGSGGSGGSGGSGGSGGSGGSGGSGGS |
| | | | GGSGGSGGSGGSGAAEGVQIQIIYFNLETV |
| | | | QVTWNASKYSRTNLTFHYRFNGDEAYDQ |
| | | | CTNYLLQEGHTSGCLLDAEQRDDILYFSIR |
| | | | NGTHPVFTASRWMVYYLKPSSPKHVRFSW |
| | | | HQDAVTVTCSDLSYGDLLYEVQYRSPFDT |
| | | | EWQSKQENTCNVTIEGLDAEKCYSFWVRV |
| | | | KAMEDVYGPDTYPSDWSEVTCWQRGEIR |
| | | | DACAETPTPPKPKLSKDIVMTQSPLSLPVTP |
| | | | GEPASISCRSSQSLLYSIGYNYLDWYLQKS |
| | | | GQSPQLLIYLGSNRASGVPDRFSGSGSGTD |
| | | | FTLKISRVEAEDVGFYYCMQALQTPYTFGQ |
| | | | GTKLEIKRTVAAPSVFIFPPSDEQLKSGTAS |
| | | | VVCLLNNFYPREAKVQWKVDNALQSGNS |
| | | | QESVTEQDSKDSTYSLSSTLTLSKADYEKH |
| | | | KVYACEVTHQGLSSPVTKSFNRGECGGGG |
| | | | SGGGGSGGGGSGGGGSESGYAQNGDLED |
| | | | AELDDYSFSCYSQLEVNGSQHSLTCAFEDP |
| | | | DVNITNLEFEICGALVEVKCLNFRKLQEIYF |
| | | | IETKKFLLIGKSNICVKVGEKSLTCKKIDLT |
| | | | TIVKPEAPFDLSVVYREGANDFVVTFNTSH |
| | | | LQKKYVKVLMHDVAYRQEKDENKWTHV |
| | | | NLSSTKLTLLQRKLQPAAMYEIKVRSIPDH |
| | | | YFKGFWSEWSPSYYFRTPEINNSSGEMDG |
| | | | GSGGSGGSGGSGGSGGSGGSGGSGGSGGS |
| | | | GGSGGSGGSGGSGGSGGSGGSGGSGGSGG |
| | | | SGAAEGVQIQIIYFNLETVQVTWNASKYSR |
| | | | TNLTFHYRFNGDEAYDQCTNYLLQEGHTS |
| | | | GCLLDAEQRDDILYFSIRNGTHPVFTASRW |
| | | | MVYYLKPSSPKHVRFSWHQDAVTVTCSDL |
| | | | SYGDLLYEVQYRSPFDTEWQSKQENTCNV |
| | | | TIEGLDAEKCYSFWVRVKAMEDVYGPDTY |
| | | | PSDWSEVTCWQRGEIRDACAETPTPPKPKL |
| | | | SK |
| | VH Domain | 10 | EVQLVESGGGLEQPGGSLRLSCAGSGFTFR |
| | | | DYAMTWVRQAPGKGLEWVSSISGSGGNT |
| | | | YYADSVKGRFTISRDNSKNTLYLQMNSLR |
| | | | AEDTAVYYCAKDRLSITIRPRYYGLDVWG |
| | | | QGTTVTVS |
| | VL Domain | 15 | DIVMTQSPLSLPVTPGEPASISCRSSQSLLYS |
| | | | IGYNYLDWYLQKSGQSPQLLIYLGSNRASG |
| | | | VPDRFSGSGSGTDFTLKISRVEAEDVGFYY |
| | | | CMQALQTPYTFGQGTKLEIK |
| | First Linker | 6 | GGGGSGGGGSGGGGSGGGGS |
| | Second Linker | 3 | GGSGGSGGSGGSGGSGGSGGSGGSGGSGG |
| | | | SGGSGGSGGSGGSGGSGGSGGSGGSGGSG |
| | | | GS |
| | HCDR1 | 7 | GFTFRDYA |
| | HCDR2 | 8 | ISGSGGNT |
| | HCDR3 | 9 | AKDRLSITIRPRYYGLDV |
| | LCDR1 | 12 | QSLLYSIGYNY |
| | LCDR2 | 13 | LGS |
| | LCDR3 | 14 | MQALQTPYT |
| | TSLP-TRAP | 5 | ESGYAQNGDLEDAELDDYSFSCYSQLEVN |
| | | | GSQHSLTCAFEDPDVNITNLEFEICGALVEV |
| | | | KCLNFRKLQEIYFIETKKFLLIGKSNICVKV |
| | | | GEKSLTCKKIDLTTIVKPEAPFDLSVVYREG |
| | | | ANDFVVTFNTSHLQKKYVKVLMHDVAYR |
| | | | QEKDENKWTHVNLSSTKLTLLQRKLQPAA |
| | | | MYEIKVRSIPDHYFKGFWSEWSPSYYFRTP |
| | | | EINNSSGEMDGGSGGSGGSGGSGGSGGSG |
| | | | GSGGSGGSGGSGGSGGSGGSGGSGGSGGS |
| | | | GGSGGSGGSGGSGAAEGVQIQIIYFNLETV |
| | | | QVTWNASKYSRTNLTFHYRFNGDEAYDQ |
| | | | CTNYLLQEGHTSGCLLDAEQRDDILYFSIR |
| | | | NGTHPVFTASRWMVYYLKPSSPKHVRFSW |
| | | | HQDAVTVTCSDLSYGDLLYEVQYRSPFDT |
| | | | EWQSKQENTCNVTIEGLDAEKCYSFWVRV |
| | | | KAMEDVYGPDTYPSDWSEVTCWQRGEIR |
| | | | DACAETPTPPKPKLSK |
| Heavy and | Full Length | 39 | ESGYAQNGDLEDAELDDYSFSCYSQLEVN |
| Light chain | Sequence | | GSQHSLTCAFEDPDVNITNLEFEICGALVEV |
| N-terminal | | | KCLNFRKLQEIYFIETKKFLLIGKSNICVKV |
| and C- | | | GEKSLTCKKIDLTTIVKPEAPFDLSVVYREG |
| terminal | | | ANDFVVTFNTSHLQKKYVKVLMHDVAYR |
| TRAP: | | | QEKDENKWTHVNLSSTKLTLLQRKLQPAA |
| TSLP | | | MYEIKVRSIPDHYFKGFWSEWSPSYYFRTP |
| TRAP | | | EINNSSGEMDGGSGGSGGSGGSGGSGGSG |
| 2_Linker- | | | GSGGSGGSGGSGGSGGSGGSGGSGGSGGS |
| GS4) | | | GGSGGSGGSGGSGAAEGVQIQIIYFNLETV |
| Dupilumab- | | | QVTWNASKYSRTNLTFHYRFNGDEAYDQ |
| YTE_Linker- | | | CTNYLLQEGHTSGCLLDAEQRDDILYFSIR |
| GS4_TSLP | | | NGTHPVFTASRWMVYYLKPSSPKHVRFSW |
| TRAP 2 [8 | | | HQDAVTVTCSDLSYGDLLYEVQYRSPFDT |
| x TRAP 2] | | | EWQSKQENTCNVTIEGLDAEKCYSFWVRV |
| | | | KAMEDVYGPDTYPSDWSEVTCWQRGEIR |
| | | | DACAETPTPPKPKLSKGGGGSGGGGSGGG |
| | | | GSGGGGSEVQLVESGGGLEQPGGSLRLSC |
| | | | AGSGFTFRDYAMTWVRQAPGKGLEWVSSI |
| | | | SGSGGNTYYADSVKGRFTISRDNSKNTLYL |
| | | | QMNSLRAEDTAVYYCAKDRLSITIRPRYYG |
| | | | LDVWGQGTTVTVSSASTKGPSVFPLAPCSR |
| | | | STSESTAALGCLVKDYFPEPVTVSWNSGAL |
| | | | TSGVHTFPAVLQSSGLYSLSSVVTVPSSSLG |
| | | | TKTYTCNVDHKPSNTKVDKRVESKYGPPC |
| | | | PPCPAPEFLGGPSVFLFPPKPKDTLYITREPE |
| | | | VTCVVVDVSQEDPEVQFNWYVDGVEVHN |
| | | | AKTKPREEQFNSTYRVVSVLTVLHQDWLN |
| | | | GKEYKCKVSNKGLPSSIEKTISKAKGQPRE |
| | | | PQVYTLPPSQEEMTKNQVSLTCLVKGFYPS |
| | | | DIAVEWESNGQPENNYKTTPPVLDSDGSFF |
| | | | LYSRLTVDKSRWQEGNVFSCSVMHEALHN |
| | | | HYTQKSLSLSLGGGGGSGGGGSGGGGSGG |
| | | | GGSESGYAQNGDLEDAELDDYSFSCYSQL |
| | | | EVNGSQHSLTCAFEDPDVNITNLEFEICGAL |
| | | | VEVKCLNFRKLQEIYFIETKKFLLIGKSNIC |
| | | | VKVGEKSLTCKKIDLTTIVKPEAPFDLSVV |
| | | | YREGANDFVVTFNTSHLQKKYVKVLMHD |
| | | | VAYRQEKDENKWTHVNLSSTKLTLLQRKL |
| | | | QPAAMYEIKVRSIPDHYFKGFWSEWSPSY |
| | | | YFRTPEINNSSGEMDGGSGGSGGSGGSGGS |
| | | | GGSGGSGGSGGSGGSGGSGGSGGSGGSGG |
| | | | SGGSGGSGGSGGSGGSGAAEGVQIQIIYFN |
| | | | LETVQVTWNASKYSRTNLTFHYRFNGDEA |
| | | | YDQCTNYLLQEGHTSGCLLDAEQRDDILY |
| | | | FSIRNGTHPVFTASRWMVYYLKPSSPKHVR |
| | | | FSWHQDAVTVTCSDLSYGDLLYEVQYRSP |
| | | | FDTEWQSKQENTCNVTIEGLDAEKCYSFW |
| | | | VRVKAMEDVYGPDTYPSDWSEVTCWQRG |
| | | | EIRDACAETPTPPKPKLSKESGYAQNGDLE |
| | | | DAELDDYSFSCYSQLEVNGSQHSLTCAFED |
| | | | PDVNITNLEFEICGALVEVKCLNFRKLQEIY |
| | | | FIETKKFLLIGKSNICVKVGEKSLTCKKIDL |
| | | | TTIVKPEAPFDLSVVYREGANDFVVTFNTS |
| | | | HLQKKYVKVLMHDVAYRQEKDENKWTH |
| | | | VNLSSTKLTLLQRKLQPAAMYEIKVRSIPD |
| | | | HYFKGFWSEWSPSYYFRTPEINNSSGEMD |
| | | | GGSGGSGGSGGSGGSGGSGGSGGSGGSGG |
| | | | SGGSGGSGGSGGSGGSGGSGGSGGSGGSG |
| | | | GSGAAEGVQIQIIYFNLETVQVTWNASKYS |
| | | | RTNLTFHYRFNGDEAYDQCTNYLLQEGHT |
| | | | SGCLLDAEQRDDILYFSIRNGTHPVFTASR |
| | | | WMVYYLKPSSPKHVRFSWHQDAVTVTCS |
| | | | DLSYGDLLYEVQYRSPFDTEWQSKQENTC |
| | | | NVTIEGLDAEKCYSFWVRVKAMEDVYGP |
| | | | DTYPSDWSEVTCWQRGEIRDACAETPTPPK |
| | | | PKLSKGGGGSGGGGSGGGGSGGGGSDIVM |
| | | | TQSPLSLPVTPGEPASISCRSSQSLLYSIGYN |
| | | | YLDWYLQKSGQSPQLLIYLGSNRASGVPD |
| | | | RFSGSGSGTDFTLKISRVEAEDVGFYYCMQ |
| | | | ALQTPYTFGQGTKLEIKRTVAAPSVFIFPPS |
| | | | DEQLKSGTASVVCLLNNFYPREAKVQWKV |
| | | | DNALQSGNSQESVTEQDSKDSTYSLSSTLT |
| | | | LSKADYEKHKVYACEVTHQGLSSPVTKSF |
| | | | NRGECGGGGSGGGGSGGGGSGGGGSESG |
| | | | YAQNGDLEDAELDDYSFSCYSQLEVNGSQ |
| | | | HSLTCAFEDPDVNITNLEFEICGALVEVKCL |
| | | | NFRKLQEIYFIETKKFLLIGKSNICVKVGEK |
| | | | SLTCKKIDLTTIVKPEAPFDLSVVYREGAN |
| | | | DFVVTFNTSHLQKKYVKVLMHDVAYRQE |
| | | | KDENKWTHVNLSSTKLTLLQRKLQPAAM |
| | | | YEIKVRSIPDHYFKGFWSEWSPSYYFRTPEI |
| | | | NNSSGEMDGGSGGSGGSGGSGGSGGSGGS |
| | | | GGSGGSGGSGGSGGSGGSGGSGGSGGSGG |
| | | | SGGSGGSGGSGAAEGVQIQIIYFNLETVQV |
| | | | TWNASKYSRTNLTFHYRFNGDEAYDQCTN |
| | | | YLLQEGHTSGCLLDAEQRDDILYFSIRNGT |
| | | | HPVFTASRWMVYYLKPSSPKHVRFSWHQ |
| | | | DAVTVTCSDLSYGDLLYEVQYRSPFDTEW |
| | | | QSKQENTCNVTIEGLDAEKCYSFWVRVKA |
| | | | MEDVYGPDTYPSDWSEVTCWQRGEIRDAC |
| | | | AETPTPPKPKLSK |
| | VH Domain | 10 | EVQLVESGGGLEQPGGSLRLSCAGSGFTFR |
| | | | DYAMTWVRQAPGKGLEWVSSISGSGGNT |
| | | | YYADSVKGRFTISRDNSKNTLYLQMNSLR |
| | | | AEDTAVYYCAKDRLSITIRPRYYGLDVWG |
| | | | QGTTVTVS |
| | VL Domain | 15 | DIVMTQSPLSLPVTPGEPASISCRSSQSLLYS |
| | | | IGYNYLDWYLQKSGQSPQLLIYLGSNRASG |
| | | | VPDRFSGSGSGTDFTLKISRVEAEDVGFYY |
| | | | CMQALQTPYTFGQGTKLEIK |
| | First Linker | 6 | GGGGSGGGGSGGGGSGGGGS |
| | Second Linker | 3 | GGSGGSGGSGGSGGSGGSGGSGGSGGSGG |
| | | | SGGSGGSGGSGGSGGSGGSGGSGGSGGSG |
| | | | GS |
| | HCDR1 | 7 | GFTFRDYA |
| | HCDR2 | 8 | ISGSGGNT |
| | HCDR3 | 9 | AKDRLSITIRPRYYGLDV |
| | LCDR1 | 12 | QSLLYSIGYNY |
| | LCDR2 | 13 | LGS |
| | LCDR3 | 14 | MQALQTPYT |
| | TSLP-TRAP | 5 | ESGYAQNGDLEDAELDDYSFSCYSQLEVN |
| | | | GSQHSLTCAFEDPDVNITNLEFEICGALVEV |
| | | | KCLNFRKLQEIYFIETKKFLLIGKSNICVKV |
| | | | GEKSLTCKKIDLTTIVKPEAPFDLSVVYREG |
| | | | ANDFVVTFNTSHLQKKYVKVLMHDVAYR |
| | | | QEKDENKWTHVNLSSTKLTLLQRKLQPAA |
| | | | MYEIKVRSIPDHYFKGFWSEWSPSYYFRTP |
| | | | EINNSSGEMDGGSGGSGGSGGSGGSGGSG |
| | | | GSGGSGGSGGSGGSGGSGGSGGSGGSGGS |
| | | | GGSGGSGGSGGSGAAEGVQIQIIYFNLETV |
| | | | QVTWNASKYSRTNLTFHYRFNGDEAYDQ |
| | | | CTNYLLQEGHTSGCLLDAEQRDDILYFSIR |
| | | | NGTHPVFTASRWMVYYLKPSSPKHVRFSW |
| | | | HQDAVTVTCSDLSYGDLLYEVQYRSPFDT |
| | | | EWQSKQENTCNVTIEGLDAEKCYSFWVRV |
| | | | KAMEDVYGPDTYPSDWSEVTCWQRGEIR |
| | | | DACAETPTPPKPKLSK |
| TSLPR (aa | | 1 | GAAEGVQIQIIYFNLETVQVTWNASKYSRT |
| 25-231) | | | NLTFHYRFNGDEAYDQCTNYLLQEGHTSG |
| | | | CLLDAEQRDDILYFSIRNGTHPVFTASRWM |
| | | | VYYLKPSSPKHVRFSWHQDAVTVTCSDLS |
| | | | YGDLLYEVQYRSPFDTEWQSKQENTCNVT |
| | | | IEGLDAEKCYSFWVRVKAMEDVYGPDTYP |
| | | | SDWSEVTCWQRGEIRDACAETPTPPKPKLS |
| | | | K |
| IL-7Rα (aa | | 2 | ESGYAQNGDLEDAELDDYSFSCYSQLEVN |
| 21-239) | | | GSQHSLTCAFEDPDVNITNLEFEICGALVEV |
| | | | KCLNFRKLQEIYFIETKKFLLIGKSNICVKV |
| | | | GEKSLTCKKIDLTTIVKPEAPFDLSVVYREG |
| | | | ANDFVVTFNTSHLQKKYVKVLMHDVAYR |
| | | | QEKDENKWTHVNLSSTKLTLLQRKLQPAA |
| | | | MYEIKVRSIPDHYFKGFWSEWSPSYYFRTP |
| | | | EINNSSGEMD |
| dupilumab- | | 11 | EVQLVESGGGLEQPGGSLRLSCAGSGFTFR |
| YTE heavy | | | DYAMTWVRQAPGKGLEWVSSISGSGGNT |
| chain | | | YYADSVKGRFTISRDNSKNTLYLQMNSLR |
| | | | AEDTAVYYCAKDRLSITIRPRYYGLDVWG |
| | | | QGTTVTVSSASTKGPSVFPLAPCSRSTSEST |
| | | | AALGCLVKDYFPEPVTVSWNSGALTSGVH |
| | | | TFPAVLQSSGLYSLSSVVTVPSSSLGTKTYT |
| | | | CNVDHKPSNTKVDKRVESKYGPPCPPCPA |
| | | | PEFLGGPSVFLFPPKPKDTLYITREPEVTCV |
| | | | VVDVSQEDPEVQFNWYVDGVEVHNAKTK |
| | | | PREEQFNSTYRVVSVLTVLHQDWLNGKEY |
| | | | KCKVSNKGLPSSIEKTISKAKGQPREPQVY |
| | | | TLPPSQEEMTKNQVSLTCLVKGFYPSDIAV |
| | | | EWESNGQPENNYKTTPPVLDSDGSFFLYSR |
| | | | LTVDKSRWQEGNVFSCSVMHEALHNHYT |
| | | | QKSLSLSLG |
| dupilumab- | | 16 | DIVMTQSPLSLPVTPGEPASISCRSSQSLLYS |
| YTE light | | | IGYNYLDWYLQKSGQSPQLLIYLGSNRASG |
| chain | | | VPDRFSGSGSGTDFTLKISRVEAEDVGFYY |
| | | | CMQALQTPYTFGQGTKLEIKRTVAAPSVFI |
| | | | FPPSDEQLKSGTASVVCLLNNFYPREAKVQ |
| | | | WKVDNALQSGNSQESVTEQDSKDSTYSLS |
| | | | STLTLSKADYEKHKVYACEVTHQGLSSPV |
| | | | TKSFNRGEC |
| Palivizumab- | | 21 | QVTLRESGPALVKPTQTLTLTCTFSGFSLST |
| YTE | | | SGMSVGWIRQPPGKALEWLADIWWDDKK |
| heavy | | | DYNPSLKSRLTISKDTSANQVVLKVTNMDP |
| chain | | | ADTATYYCARSMITNWYFDVWGAGTTVT |
| | | | VSSASTKGPSVFPLAPCSRSTSESTAALGCL |
| | | | VKDYFPEPVTVSWNSGALTSGVHTFPAVL |
| | | | QSSGLYSLSSVVTVPSSSLGTKTYTCNVDH |
| | | | KPSNTKVDKRVESKYGPPCPPCPAPEFLGG |
| | | | PSVFLFPPKPKDTLYITREPEVTCVVVDVSQ |
| | | | EDPEVQFNWYVDGVEVHNAKTKPREEQF |
| | | | NSTYRVVSVLTVLHQDWLNGKEYKCKVS |
| | | | NKGLPSSIEKTISKAKGQPREPQVYTLPPSQ |
| | | | EEMTKNQVSLTCLVKGFYPSDIAVEWESN |
| | | | GQPENNYKTTPPVLDSDGSFFLYSRLTVDK |
| | | | SRWQEGNVFSCSVMHEALHNHYTQKSLSL |
| | | | SLG |
| Palivizumab- | | 26 | DIQMTQSPSTLSASVGDRVTITCKCQLSVG |
| YTE | | | YMHWYQQKPGKAPKLLIYDTSKLASGVPS |
| light chain | | | RFSGSGSGTAFTLTISSLQPDDFATYYCFQG |
| | | | SGYPFTFGGGTKLEIKRTVAAPSVFIFPPSD |
| | | | EQLKSGTASVVCLLNNFYPREAKVQWKVD |
| | | | NALQSGNSQESVTEQDSKDSTYSLSSTLTL |
| | | | SKADYEKHKVYACEVTHQGLSSPVTKSFN |
| | | | RGEC |
| Flexible | | 40 | (GGGGS)n |
| Linker | | | |
| Flexible | | 41 | (EAAAK)n |
| Linker | | | |
| Flexible | | 42 | (SGGGG)n |
| Linker | | | |
| Flexible | | 43 | GGGG(SGGGG)n |
| Linker | | | |
| Flexible | | 44 | GG(SGG)n |
| Linker | | | |
| A variant | Full length | 45 | EVQLVESGGGLEQPGGSLRLSCAGSGFTFR |
| of Light | sequence | | DYAMTWVRQAPGKGLEWVSSISGSGGNT |
| chain C- | | | YYADSVKGRFTISRDNSKNTLYLQMNSLR |
| terminal | | | AEDTAVYYCAKDRLSITIRPRYYGLDVWG |
| TRAP: | | | QGTTVTVSSASTKGPSVFPLAPCSRSTSEST |
| Dupilumab- | | | AALGCLVKDYFPEPVTVSWNSGALTSGVH |
| YTE Linker- | | | TFPAVLQSSGLYSLSSVVTVPSSSLGTKTYT |
| GS4_TSLP | | | CNVDHKPSNTKVDKRVESKYGPPCPPCPA |
| TRAP 2 [2 | | | PEFLGGPSVFLFPPKPKDTLYITREPEVTCV |
| x TRAP 2] | | | VVDVSQEDPEVQFNWYVDGVEVHNAKTK |
| | | | PREEQFNSTYRVVSVLTVLHQDWLNGKEY |
| | | | KCKVSNKGLPSSIEKTISKAKGQPREPQVY |
| | | | TLPPSQEEMTKNQVSLTCLVKGFYPSDIAV |
| | | | EWESNGQPENNYKTTPPVLDSDGSFFLYSR |
| | | | LTVDKSRWQEGNVFSCSVMHEALHNHYT |
| | | | QKSLSLSLGDIVMTQSPLSLPVTPGEPASIS |
| | | | CRSSQSLLYSIGYNYLDWYLQKSGQSPQLL |
| | | | IYLGSNRASGVPDRFSGSGSGTDFTLKISRV |
| | | | EAEDVGFYYCMQALQTPYTFGQGTKLEIK |
| | | | RTVAAPSVFIFPPSDEQLKSGTASVVCLLN |
| | | | NFYPREAKVQWKVDNALQSGNSQESVTEQ |
| | | | DSKDSTYSLSSTLTLSKADYEKHKVYACEV |
| | | | THQGLSSPVTKSFNRGECGGGGSGGGGSG |
| | | | GGGSGGGGSESGYAQNGDLEDAELDDYSF |
| | | | SCYSQLEVNGSQHSLTCAFEDPDVNITNLE |
| | | | FEICGALVEVKCLNFRKLQEIYFIETKKFLLI |
| | | | GKSNICVKVGEKSLTCKKIDLTTIVKPEAPF |
| | | | DLSVVYREGANDFVVTFNTSHLQKKYVKV |
| | | | LMHDVAYRQEKDENKWTHVNLSSTKLTL |
| | | | LQRKLQPAAMYEIKVRSIPDHYFKGFWSE |
| | | | WSPSYYFRTPEINNSSGEMD |
| A variant | Full length | 46 | ESGYAQNGDLEDAELDDYSFSCYSQLEVN |
| of Heavy | sequence | | GSQHSLTCAFEDPDVNITNLEFEICGALVEV |
| and Light | | | KCLNFRKLQEIYFIETKKFLLIGKSNICVKV |
| chain C- | | | GEKSLTCKKIDLTTIVKPEAPFDLSVVYREG |
| terminal | | | ANDFVVTFNTSHLQKKYVKVLMHDVAYR |
| TRAP: | | | QEKDENKWTHVNLSSTKLTLLQRKLQPAA |
| Dupilumab- | | | MYEIKVRSIPDHYFKGFWSEWSPSYYFRTP |
| YTE Linker- | | | EINNSSGEMDGGSGGSGGSGGSGGSGGSG |
| GS4_TSLP | | | GSGGSGGSGGSGGSGGSGGSGGSGGSGGS |
| TRAP 2 [4 | | | GGSGGSGGSGGSGAAEGVQIQIIYFNLETV |
| x TRAP 2] | | | QVTWNASKYSRTNLTFHYRFNGDEAYDQ |
| | | | CTNYLLQEGHTSGCLLDAEQRDDILYFSIR |
| | | | NGTHPVFTASRWMVYYLKPSSPKHVRFSW |
| | | | HQDAVTVTCSDLSYGDLLYEVQYRSPFDT |
| | | | EWQSKQENTCNVTIEGLDAEKCYSFWVRV |
| | | | KAMEDVYGPDTYPSDWSEVTCWQRGEIR |
| | | | DACAETPTPPKPKLSKGGGGSGGGGSGGG |
| | | | GSGGGGSEVQLVESGGGLEQPGGSLRLSC |
| | | | AGSGFTFRDYAMTWVRQAPGKGLEWVSSI |
| | | | SGSGGNTYADSVKGRFTISRDNSKNTLYLQ |
| | | | MNSLRAEDTAVYYCAKDRLSITIRPRYYGL |
| | | | DVWGQGTTVTVSSASTKGPSVFPLAPCSRS |
| | | | TSESTAALGCLVKDYFPEPVTVSWNSGALT |
| | | | SGVHTFPAVLQSSGLYSLSSVVTVPSSSLGT |
| | | | KTYTCNVDHKPSNTKVDKRVESKYGPPCP |
| | | | PCPAPEFLGGPSVFLFPPKPKDTLYITREPE |
| | | | VTCVVVDVSQEDPEVQFNWYVDGVEVHN |
| | | | AKTKPREEQFNSTYRVVSVLTVLHQDWLN |
| | | | GKEYKCKVSNKGLPSSIEKTISKAKGQPRE |
| | | | PQVYTLPPSQEEMTKNQVSLTCLVKGFYPS |
| | | | DIAVEWESNGQPENNYKTTPPVLDSDGSFF |
| | | | LYSRLTVDKSRWQEGNVFSCSVMHEALHN |
| | | | HYTQKSLSLSLGESGYAQNGDLEDAELDD |
| | | | YSFSCYSQLEVNGSQHSLTCAFEDPDVNIT |
| | | | NLEFEICGALVEVKCLNFRKLQEIYFIETKK |
| | | | FLLIGKSNICVKVGEKSLTCKKIDLTTIVKP |
| | | | EAPFDLSVVYREGANDFVVTFNTSHLQKK |
| | | | YVKVLMHDVAYRQEKDENKWTHVNLSST |
| | | | KLTLLQRKLQPAAMYEIKVRSIPDHYFKGF |
| | | | WSEWSPSYYFRTPEINNSSGEMDGGSGGS |
| | | | GGSGGSGGSGGSGGSGGSGGSGGSGGSGG |
| | | | SGGSGGSGGSGGSGGSGGSGGSGGSGAAE |
| | | | GVQIQIIYFNLETVQVTWNASKYSRTNLTF |
| | | | HYRFNGDEAYDQCTNYLLQEGHTSGCLLD |
| | | | AEQRDDILYFSIRNGTHPVFTASRWMVYYL |
| | | | KPSSPKHVRFSWHQDAVTVTCSDLSYGDL |
| | | | LYEVQYRSPFDTEWQSKQENTCNVTIEGLD |
| | | | AEKCYSFWVRVKAMEDVYGPDTYPSDWS |
| | | | EVTCWQRGEIRDACAETPTPPKPKLSKGGG |
| | | | GSGGGGSGGGGSGGGGSDIVMTQSPLSLP |
| | | | VTPGEPASISCRSSQSLLYSIGYNYLDWYLQ |
| | | | KSGQSPQLLIYLGSNRASGVPDRFSGSGSG |
| | | | TDFTLKISRVEAEDVGFYYCMQALQTPYTF |
| | | | GQGTKLEIKRTVAAPSVFIFPPSDEQLKSGT |
| | | | ASVVCLLNNFYPREAKVQWKVDNALQSG |
| | | | NSQESVTEQDSKDSTYSLSSTLTLSKADYE |
| | | | KHKVYACEVTHQGLSSPVTKSFNRGEC |
| A variant | Full length | 47 | EVQLVESGGGLEQPGGSLRLSCAGSGFTFR |
| of Heavy | sequence | | DYAMTWVRQAPGKGLEWVSSISGSGGNT |
| and Light | | | YYADSVKGRFTISRDNSKNTLYLQMNSLR |
| chain C- | | | AEDTAVYYCAKDRLSITIRPRYYGLDVWG |
| terminal | | | QGTTVTVSSASTKGPSVFPLAPCSRSTSEST |
| TRAP: | | | AALGCLVKDYFPEPVTVSWNSGALTSGVH |
| Dupilumab- | | | TFPAVLQSSGLYSLSSVVTVPSSSLGTKTYT |
| YTE Linker- | | | CNVDHKPSNTKVDKRVESKYGPPCPPCPA |
| GS4_TSLP | | | PEFLGGPSVFLFPPKPKDTLYITREPEVTCV |
| TRAP 2 [4 | | | VVDVSQEDPEVQFNWYVDGVEVHNAKTK |
| x TRAP 2] | | | PREEQFNSTYRVVSVLTVLHQDWLNGKEY |
| | | | KCKVSNKGLPSSIEKTISKAKGQPREPQVY |
| | | | TLPPSQEEMTKNQVSLTCLVKGFYPSDIAV |
| | | | EWESNGQPENNYKTTPPVLDSDGSFFLYSR |
| | | | LTVDKSRWQEGNVFSCSVMHEALHNHYT |
| | | | QKSLSLSLGGGGGSGGGGSGGGGSGGGGS |
| | | | ESGYAQNGDLEDAELDDYSFSCYSQLEVN |
| | | | GSQHSLTCAFEDPDVNITNLEFEICGALVEV |
| | | | KCLNFRKLQEIYFIETKKFLLIGKSNICVKV |
| | | | GEKSLTCKKIDLTTIVKPEAPFDLSVVYREG |
| | | | ANDFVVTFNTSHLQKKYVKVLMHDVAYR |
| | | | QEKDENKWTHVNLSSTKLTLLQRKLQPAA |
| | | | MYEIKVRSIPDHYFKGFWSEWSPSYYFRTP |
| | | | EINNSSGEMDGGSGGSGGSGGSGGSGGSG |
| | | | GSGGSGGSGGSGGSGGSGGSGGSGGSGGS |
| | | | GGSGGSGGSGGSGAAEGVQIQIIYFNLETV |
| | | | QVTWNASKYSRTNLTFHYRFNGDEAYDQ |
| | | | CTNYLLQEGHTSGCLLDAEQRDDILYFSIR |
| | | | NGTHPVFTASRWMVYYLKPSSPKHVRFSW |
| | | | HQDAVTVTCSDLSYGDLLYEVQYRSPFDT |
| | | | EWQSKQENTCNVTIEGLDAEKCYSFWVRV |
| | | | KAMEDVYGPDTYPSDWSEVTCWQRGEIR |
| | | | DACAETPTPPKPKLSKDIVMTQSPLSLPVTP |
| | | | GEPASISCRSSQSLLYSIGYNYLDWYLQKS |
| | | | GQSPQLLIYLGSNRASGVPDRFSGSGSGTD |
| | | | FTLKISRVEAEDVGFYYCMQALQTPYTFGQ |
| | | | GTKLEIKRTVAAPSVFIFPPSDEQLKSGTAS |
| | | | VVCLLNNFYPREAKVQWKVDNALQSGNS |
| | | | QESVTEQDSKDSTYSLSSTLTLSKADYEKH |
| | | | KVYACEVTHQGLSSPVTKSFNRGECGGGG |
| | | | SGGGGSGGGGSGGGGSESGYAQNGDLED |
| | | | AELDDYSFSCYSQLEVNGSQHSLTCAFEDP |
| | | | DVNITNLEFEICGALVEVKCLNFRKLQEIYF |
| | | | IETKKFLLIGKSNICVKVGEKSLTCKKIDLT |
| | | | TIVKPEAPFDLSVVYREGANDFVVTFNTSH |
| | | | LQKKYVKVLMHDVAYRQEKDENKWTHV |
| | | | NLSSTKLTLLQRKLQPAAMYEIKVRSIPDH |
| | | | YFKGFWSEWSPSYYFRTPEINNSSGEMD |
| Candidate | Full length | 48 | EVQLVESGGGLEQPGGSLRLSCAGSGFTFR |
| Sequence: | sequence | | DYAMTWVRQAPGKGLEWVSSISGSGGNT |
| Dupilumab | | | YYADSVKGRFTISRDNSKNTLYLQMNSLR |
| YTE | | | AEDTAVYYCAKDRLSITIRPRYYGLDVWG |
| | | | QGTTVTVSSASTKGPSVFPLAPCSRSTSEST |
| | | | AALGCLVKDYFPEPVTVSWNSGALTSGVH |
| | | | TFPAVLQSSGLYSLSSVVTVPSSSLGTKTYT |
| | | | CNVDHKPSNTKVDKRVESKYGPPCPPCPA |
| | | | PEFLGGPSVFLFPPKPKDTLYITREPEVTCV |
| | | | VVDVSQEDPEVQFNWYVDGVEVHNAKTK |
| | | | PREEQFNSTYRVVSVLTVLHQDWLNGKEY |
| | | | KCKVSNKGLPSSIEKTISKAKGQPREPQVY |
| | | | TLPPSQEEMTKNQVSLTCLVKGFYPSDIAV |
| | | | EWESNGQPENNYKTTPPVLDSDGSFFLYSR |
| | | | LTVDKSRWQEGNVFSCSVMHEALHNHYT |
| | | | QKSLSLSLGDIVMTQSPLSLPVTPGEPASIS |
| | | | CRSSQSLLYSIGYNYLDWYLQKSGQSPQLL |
| | | | IYLGSNRASGVPDRFSGSGSGTDFTLKISRV |
| | | | EAEDVGFYYCMQALQTPYTFGQGTKLEIK |
| | | | RTVAAPSVFIFPPSDEQLKSGTASVVCLLN |
| | | | NFYPREAKVQWKVDNALQSGNSQESVTEQ |
| | | | DSKDSTYSLSSTLTLSKADYEKHKVYACEV |
| | | | THQGLSSPVTKSFNRGEC |
| A variant | Full sequence | 49 | QVTLRESGPALVKPTQTLTLTCTFSGFSLST |
| of | | | SGMSVGWIRQPPGKALEWLADIWWDDKK |
| Palivizumab- | | | DYNPSLKSRLTISKDTSANQVVLKVTNMDP |
| YTE_Linker- | | | ADTATYYCARSMITNWYFDVWGAGTTVT |
| GS4_TSLP | | | VSSASTKGPSVFPLAPCSRSTSESTAALGCL |
| TRAP 1 | | | VKDYFPEPVTVSWNSGALTSGVHTFPAVL |
| | | | QSSGLYSLSSVVTVPSSSLGTKTYTCNVDH |
| | | | KPSNTKVDKRVESKYGPPCPPCPAPEFLGG |
| | | | PSVFLFPPKPKDTLYITREPEVTCVVVDVSQ |
| | | | EDPEVQFNWYVDGVEVHNAKTKPREEQF |
| | | | NSTYRVVSVLTVLHQDWLNGKEYKCKVS |
| | | | NKGLPSSIEKTISKAKGQPREPQVYTLPPSQ |
| | | | EEMTKNQVSLTCLVKGFYPSDIAVEWESN |
| | | | GQPENNYKTTPPVLDSDGSFFLYSRLTVDK |
| | | | SRWQEGNVFSCSVMHEALHNHYTQKSLSL |
| | | | SLGGGGGSGGGGSGGGGSGGGGSGAAEG |
| | | | VQIQIIYFNLETVQVTWNASKYSRTNLTFH |
| | | | YRFNGDEAYDQCTNYLLQEGHTSGCLLDA |
| | | | EQRDDILYFSIRNGTHPVFTASRWMVYYLK |
| | | | PSSPKHVRFSWHQDAVTVTCSDLSYGDLL |
| | | | YEVQYRSPFDTEWQSKQENTCNVTIEGLD |
| | | | AEKCYSFWVRVKAMEDVYGPDTYPSDWS |
| | | | EVTCWQRGEIRDACAETPTPPKPKLSKGGS |
| | | | GGSGGSGGSGGSGGSGGSGGSGGSGGSGG |
| | | | SGGSGGSGGSGGSGGSGGSGGSGGSGGSE |
| | | | SGYAQNGDLEDAELDDYSFSCYSQLEVNG |
| | | | SQHSLTCAFEDPDVNITNLEFEICGALVEVK |
| | | | CLNFRKLQEIYFIETKKFLLIGKSNICVKVG |
| | | | EKSLTCKKIDLTTIVKPEAPFDLSVVYREGA |
| | | | NDFVVTFNTSHLQKKYVKVLMHDVAYRQ |
| | | | EKDENKWTHVNLSSTKLTLLQRKLQPAAM |
| | | | YEIKVRSIPDHYFKGFWSEWSPSYYFRTPEI |
| | | | NNSSGEMDIQMTQSPSTLSASVGDRVTITC |
| | | | KCQLSVGYMHWYQQKPGKAPKLLIYDTS |
| | | | KLASGVPSRFSGSGSGTAFTLTISSLQPDDF |
| | | | ATYYCFQGSGYPFTFGGGTKLEIKRTVAAP |
| | | | SVFIFPPSDEQLKSGTASVVCLLNNFYPREA |
| | | | KVQWKVDNALQSGNSQESVTEQDSKDST |
| | | | YSLSSTLTLSKADYEKHKVYACEVTHQGL |
| | | | SSPVTKSFNRGEC |
| A variant | Full Length | 50 | GAAEGVQIQIIYFNLETVQVTWNASKYSRT |
| of TSLP | Sequence | | NLTFHYRFNGDEAYDQCTNYLLQEGHTSG |
| TRAP | | | CLLDAEQRDDILYFSIRNGTHPVFTASRWM |
| 1_Linker- | | | VYYLKPSSPKHVRFSWHQDAVTVTCSDLS |
| GS4_ | | | YGDLLYEVQYRSPFDTEWQSKQENTCNVT |
| Dupilumab- | | | IEGLDAEKCYSFWVRVKAMEDVYGPDTYP |
| YTE | | | SDWSEVTCWQRGEIRDACAETPTPPKPKLS |
| | | | KGGSGGSGGSGGSGGSGGSGGSGGSGGSG |
| | | | GSGGSGGSGGSGGSGGSGGSGGSGGSGGS |
| | | | GGSESGYAQNGDLEDAELDDYSFSCYSQL |
| | | | EVNGSQHSLTCAFEDPDVNITNLEFEICGAL |
| | | | VEVKCLNFRKLQEIYFIETKKFLLIGKSNIC |
| | | | VKVGEKSLTCKKIDLTTIVKPEAPFDLSVV |
| | | | YREGANDFVVTFNTSHLQKKYVKVLMHD |
| | | | VAYRQEKDENKWTHVNLSSTKLTLLQRKL |
| | | | QPAAMYEIKVRSIPDHYFKGFWSEWSPSY |
| | | | YFRTPEINNSSGEMDGGGGSGGGGSGGGG |
| | | | SGGGGSVQLVESGGGLEQPGGSLRLSCAGS |
| | | | GFTFRDYAMTWVRQAPGKGLEWVSSISGS |
| | | | GGNTYYADSVKGRFTISRDNSKNTLYLQM |
| | | | NSLRAEDTAVYYCAKDRLSITIRPRYYGLD |
| | | | VWGQGTTVTVSSASTKGPSVFPLAPCSRST |
| | | | SESTAALGCLVKDYFPEPVTVSWNSGALTS |
| | | | GVHTFPAVLQSSGLYSLSSVVTVPSSSLGT |
| | | | KTYTCNVDHKPSNTKVDKRVESKYGPPCP |
| | | | PCPAPEFLGGPSVFLFPPKPKDTLYITREPE |
| | | | VTCVVVDVSQEDPEVQFNWYVDGVEVHN |
| | | | AKTKPREEQFNSTYRVVSVLTVLHQDWLN |
| | | | GKEYKCKVSNKGLPSSIEKTISKAKGQPRE |
| | | | PQVYTLPPSQEEMTKNQVSLTCLVKGFYPS |
| | | | DIAVEWESNGQPENNYKTTPPVLDSDGSFF |
| | | | LYSRLTVDKSRWQEGNVFSCSVMHEALHN |
| | | | HYTQKSLSLSLGDIVMTQSPLSLPVTPGEPA |
| | | | SISCRSSQSLLYSIGYNYLDWYLQKSGQSP |
| | | | QLLIYLGSNRASGVPDRFSGSGSGTDFTLKI |
| | | | SRVEAEDVGFYYCMQALQTPYTFGQGTKL |
| | | | EIKRTVAAPSVFIFPPSDEQLKSGTASVVCL |
| | | | LNNFYPREAKVQWKVDNALQSGNSQESVT |
| | | | EQDSKDSTYSLSSTLTLSKADYEKHKVYCE |
| | | | VTHQGLSSPVTKSFNRGEC |

1.-135. (canceled)

136. An antigen binding complex comprising a TSLP-TRAP and a binding moiety comprising a heavy chain, wherein the TSLP-TRAP is associated with a C-terminal of the heavy chain via a first linker, wherein the TSLP-TRAP comprises an extracellular domain of thymic stromal lymphopoietin protein receptor (TSLPR) and an extracellular domain of interleukin-7 receptor subunit alpha (IL-7Rα) joined via a second linker.

137. The antigen binding complex of claim 136, wherein the TSLP-TRAP comprises a sequence having at least 80% sequence identity to the amino acid sequence of SEQ ID NO: 4.

138. The antigen binding complex of claim 136, wherein the TSLP-TRAP comprises a sequence having at least 80% sequence identity to the amino acid sequence of SEQ ID NO: 5.

139. The antigen binding complex of claim 136, wherein the binding moiety is an antibody or antigen binding portion thereof.

140. The antigen binding complex of claim 139, wherein the antibody or antigen binding portion thereof is a monoclonal antibody, a bispecific antibody, a Fab, a Fab′, a Fv, a scFV, or a (Fab′)2.

141. The antigen binding complex of claim 139, wherein the antibody or antigen binding portion thereof specifically binds human interleukin-4 receptor (hIL-4R), human interleukin-4 (hIL-4), human interleukin-13 receptor (hIL-13R), human interleukin-13 (hIL-13), human interleukin-5 (hIL-5), human interleukin-5 receptor (hIL-5R), human interleukin-33 (hIL-33) or human interleukin-31 (hIL-31).

142. The antigen binding complex of claim 141, wherein the antibody or antigen-binding portion thereof specifically binds human interleukin-4 receptor (hIL-4R).

143. The antigen binding complex of claim 139, wherein the antibody or antigen binding portion thereof comprises a Fc region having one or more mutations to extend half-life.

144. The antigen binding complex of claim 143, wherein the one or more mutations to extend half-life comprises M252Y/S254T/T256E (YTE), M428L/N434S (LS), T307A/E380A/N434A (AAA), T250Q/M428L (QL), or V308P sequence mutations.

145. The antigen binding complex of claim 139, wherein the antibody or antigen binding portion thereof comprises (a) an LCDR1 having at least 80% sequence identity to the amino acid sequence of SEQ ID NO: 12, (b) an LCDR2 having at least 80% sequence identity to the amino acid sequence of SEQ ID NO: 13, and/or (c) an LCDR3 having at least 80% sequence identity to the amino acid sequence of SEQ ID NO: 14.

146. The antigen binding complex of claim 145, wherein the antibody or antigen binding portion thereof comprises (a) an HCDR1 having at least 80% sequence identity to the amino acid sequence of SEQ ID NO: 7, (b) an HCDR2 having at least 80% sequence identity to the amino acid sequence of SEQ ID NO: 8, and/or (c) an HCDR3 having at least 80% sequence identity to the amino acid sequence of SEQ ID NO: 9.

147. The antigen binding complex of claim 136, wherein the antigen binding complex comprises a sequence having at least 80% sequence identity to the amino acid sequence of SEQ ID NO: 33.

148. The antigen binding complex of claim 147, wherein the antigen binding complex comprises the amino acid sequence of SEQ ID NO: 33.

149. The antigen binding complex of claim 136, wherein the antigen binding complex comprises a sequence having at least 80% sequence identity to the amino acid sequence of SEQ ID NO: 34.

150. The antigen binding complex of claim 149, wherein the antigen binding complex comprises the amino acid sequence of SEQ ID NO: 34.

151. A method of treating a disease or disorder in a subject in need thereof, the method comprising administering to the subject an antigen binding complex comprising a TSLP-TRAP and a binding moiety comprising a heavy chain, wherein the TSLP-TRAP is associated with a C-terminal of the heavy chain via a first linker and the TSLP-TRAP comprises an extracellular domain of thymic stromal lymphopoietin protein receptor (TSLPR) and an extracellular domain of interleukin-7 receptor subunit alpha (IL-7Rα) joined via a second linker.

152. The method of claim 151, wherein the antigen binding complex comprises a sequence having at least 80% sequence identity to the amino acid sequence of SEQ ID NO: 33.

153. The method of claim 152, wherein the antigen binding complex comprises the amino acid sequence of SEQ ID NO: 33.

154. The method of claim 151, wherein the antigen binding complex comprises a sequence having at least 80% sequence identity to the amino acid sequence of SEQ ID NO: 34.

155. The method of claim 154, wherein the antigen binding complex comprises the amino acid sequence of SEQ ID NO: 34.


SEQUENCE LISTING (50 sequences)

SEQ ID NO: 1 - protein, 207 aa, synthetic construct
GAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSK

SEQ ID NO: 2 - protein, 219 aa, synthetic construct
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMD

SEQ ID NO: 3 - protein, 60 aa, synthetic construct
GGSGGSGGSGGSGGSGGSGGSGGSGGSGGSGGSGGSGGSGGSGGSGGSGGSGGSGGSGGS

SEQ ID NO: 4 - protein, 486 aa, synthetic construct
GAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKGGSGGSGGSGGSGGSGGSGGSGGSGGSGGSGGSGGSGGSGGSGGSGGSGGSGGSGGSGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMD

SEQ ID NO: 5 - protein, 486 aa, synthetic construct
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSK

SEQ ID NO: 6 - protein, 20 aa, synthetic construct
GGGGSGGGGSGGGGSGGGGS

SEQ ID NO: 7 - protein, 8 aa, synthetic construct
GFTFRDYA

SEQ ID NO: 8 - protein, 8 aa, synthetic construct
ISGSGGNT

SEQ ID NO: 9 - protein, 18 aa, synthetic construct
AKDRLSITIRPRYYGLDV

SEQ ID NO: 10 - protein, 124 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 11 - protein, 451 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 12 - protein, 11 aa, synthetic construct
QSLLYSIGYNY

SEQ ID NO: 13 - skipped

SEQ ID NO: 14 - protein, 9 aa, synthetic construct
MQALQTPYT

SEQ ID NO: 15 - protein, 112 aa, synthetic construct [sequence omitted]

SEQ ID NO: 16 - protein, 219 aa, synthetic construct
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 10 aa, synthetic construct
GFSLSTSGMS

SEQ ID NO: 18 - protein, 7 aa, synthetic construct
IWWDDKK

SEQ ID NO: 19 - protein, 12 aa, synthetic construct
ARSMITNWYFDV

SEQ ID NO: 20 - protein, 120 aa, synthetic construct
QVTLRESGPALVKPTQTLTLTCTFSGFSLSTSGMSVGWIRQPPGKALEWLADIWWDDKKDYNPSLKSRLTISKDTSANQVVLKVTNMDPADTATYYCARSMITNWYFDVWGAGTTVTVSS

SEQ ID NO: 21 - protein, 446 aa, synthetic construct
QVTLRESGPALVKPTQTLTLTCTFSGFSLSTSGMSVGWIRQPPGKALEWLADIWWDDKKDYNPSLKSRLTISKDTSANQVVLKVTNMDPADTATYYCARSMITNWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 22 - protein, 5 aa, synthetic construct
LSVGY

SEQ ID NO: 23 - skipped

SEQ ID NO: 24 - protein, 9 aa, synthetic construct
FQGSGYPFT

SEQ ID NO: 25 - protein, 106 aa, synthetic construct
DIQMTQSPSTLSASVGDRVTITCKCQLSVGYMHWYQQKPGKAPKLLIYDTSKLASGVPSRFSGSGSGTAFTLTISSLQPDDFATYYCFQGSGYPFTFGGGTKLEIK

SEQ ID NO: 26 - protein, 213 aa, synthetic construct
DIQMTQSPSTLSASVGDRVTITCKCQLSVGYMHWYQQKPGKAPKLLIYDTSKLASGVPSRFSGSGSGTAFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 1165 aa, synthetic construct
QVTLRESGPALVKPTQTLTLTCTFSGFSLSTSGMSVGWIRQPPGKALEWLADIWWDDKKDYNPSLKSRLTISKDTSANQVVLKVTNMDPADTATYYCARSMITNWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKGGSGGSGGSGGSGGSGGSGGSGGSGGSGGSGGSGGSGGSGGSGGSGGSGGSGGSGGSGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDDIQMTQSPSTLSASVGDRVTITCKCQLSVGYMHWYQQKPGKAPKLLIYDTSKLASGVPSRFSGSGSGTAFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 1165 aa, synthetic construct
QVTLRESGPALVKPTQTLTLTCTFSGFSLSTSGMSVGWIRQPPGKALEWLADIWWDDKKDYNPSLKSRLTISKDTSANQVVLKVTNMDPADTATYYCARSMITNWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKDIQMTQSPSTLSASVGDRVTITCKCQLSVGYMHWYQQKPGKAPKLLIYDTSKLASGVPSRFSGSGSGTAFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - protein, 1165 aa, synthetic construct
GAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKGGSGGSGGSGGSGGSGGSGGSGGSGGSGGSGGSGGSGGSGGSGGSGGSGGSGGSGGSGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGGGSGGGGSGGGGSGGGGSQVTLRESGPALVKPTQTLTLTCTFSGFSLSTSGMSVGWIRQPPGKALEWLADIWWDDKKDYNPSLKSRLTISKDTSANQVVLKVTNMDPADTATYYCARSMITNWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDIQMTQSPSTLSASVGDRVTITCKCQLSVGYMHWYQQKPGKAPKLLIYDTSKLASGVPSRFSGSGSGTAFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 1165 aa, synthetic construct
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKGGGGSGGGGSGGGGSGGGGSQVTLRESGPALVKPTQTLTLTCTFSGFSLSTSGMSVGWIRQPPGKALEWLADIWWDDKKDYNPSLKSRLTISKDTSANQVVLKVTNMDPADTATYYCARSMITNWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDIQMTQSPSTLSASVGDRVTITCKCQLSVGYMHWYQQKPGKAPKLLIYDTSKLASGVPSRFSGSGSGTAFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 1176 aa, synthetic construct
GAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKGGSGGSGGSGGSGGSGGSGGSGGSGGSGGSGGSGGSGGSGGSGGSGGSGGSGGSGGSGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGGGSGGGGSGGGGSGGGGSEVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 1176 aa, synthetic construct
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKGGGGSGGGGSGGGGSGGGGSEVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 1176 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKGGSGGSGGSGGSGGSGGSGGSGGSGGSGGSGGSGGSGGSGGSGGSGGSGGSGGSGGSGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDDIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 1176 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKDIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 1176 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKGGGGSGGGGSGGGGSGGGGSDIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 1176 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSK

SEQ ID NO: 37 - protein, 1682 aa, synthetic construct
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKGGGGSGGGGSGGGGSGGGGSEVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKGGGGSGGGGSGGGGSGGGGSDIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 1682 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKDIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSK

SEQ ID NO: 39 - protein, 2694 aa, synthetic construct
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKGGGGSGGGGSGGGGSGGGGSEVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKGGGGSGGGGSGGGGSGGGGSDIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSK

SEQ ID NO: 40 - protein, 500 aa, synthetic construct
  VARIANT 1..500: note = This sequence may encompass 1-100 GGGGS repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 41 - protein, 500 aa, synthetic construct
  VARIANT 1..500: note = This sequence may encompass 1-100 EAAAK repeating units
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 42 - protein, 500 aa, synthetic construct
  VARIANT 1..500: note = This sequence may encompass 1-100 SGGGG repeating units
SGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGG

SEQ ID NO: 43 - protein, 504 aa, synthetic construct
  VARIANT 5..504: note = This region may encompass 1-100 SGGGG repeating units
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGG

SEQ ID NO: 44 - protein, 302 aa, synthetic construct
  VARIANT 3..302: note = This region may encompass 1-100 SGG repeating units
GGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGGSGG

SEQ ID NO: 45 - protein, 909 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMD

SEQ ID NO: 46 - protein, 1681 aa, synthetic construct
ESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKGGGGSGGGGSGGGGSGGGGSEVQLVESGGGLEQPGGSLRLSCAGSGFTFRDYAMTWVRQAPGKGLEWVSSISGSGGNTYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKGGGGSGGGGSGGGGSGGGGSDIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 1415 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGSGGSGGSGGSGGSGGSGGSGGSGGSGGSGGSGGSGGSGGSGGSGGSGGSGGSGGSGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKDIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSGGGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMD

SEQ ID NO: 48 - protein, 670 aa, synthetic construct
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 1164 aa, synthetic construct
QVTLRESGPALVKPTQTLTLTCTFSGFSLSTSGMSVGWIRQPPGKALEWLADIWWDDKKDYNPSLKSRLTISKDTSANQVVLKVTNMDPADTATYYCARSMITNWYFDVWGAGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGSGGGGSGAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKGGSGGSGGSGGSGGSGGSGGSGGSGGSGGSGGSGGSGGSGGSGGSGGSGGSGGSGGSGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDIQMTQSPSTLSASVGDRVTITCKCQLSVGYMHWYQQKPGKAPKLLIYDTSKLASGVPSRFSGSGSGTAFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 1174 aa, synthetic construct
GAAEGVQIQIIYFNLETVQVTWNASKYSRTNLTFHYRFNGDEAYDQCTNYLLQEGHTSGCLLDAEQRDDILYFSIRNGTHPVFTASRWMVYYLKPSSPKHVRFSWHQDAVTVTCSDLSYGDLLYEVQYRSPFDTEWQSKQENTCNVTIEGLDAEKCYSFWVRVKAMEDVYGPDTYPSDWSEVTCWQRGEIRDACAETPTPPKPKLSKGGSGGSGGSGGSGGSGGSGGSGGSGGSGGSGGSGGSGGSGGSGGSGGSGGSGGSGGSGGSESGYAQNGDLEDAELDDYSFSCYSQLEVNGSQHSLTCAFEDPDVNITNLEFEICGALVEVKCLNFRKLQEIYFIETKKFLLIGKSNICVKVGEKSLTCKKIDLTTIVKPEAPFDLSVVYREGANDFVVTFNTSHLQKKYVKVLMHDVAYRQEKDENKWTHVNLSSTKLTLLQRKLQPAAMYEIKVRSIPDHYFKGFWSEWSPSYYFRTPEINNSSGEMDGGGGSGGGGSGGGGSGGGGSVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGDIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYCEVTHQGLSSPVTKSFNRGEC